SEQUENCE LISTING (49 sequences)

SEQ ID NO: 1 - DNA, 1572 bp, Nepenthes kassiana
atgaatgctccgtgcttctgcttccatgcacaaaaaatgcgaaaccacaagcacagtaccatgaggggttgggtagtgcttctgctgctcaatttacctttcctttcagcattccaatgcggccaacaagccggtggagcgctgtgccacagtggactctgctgcagccagtggggttggtgtggtaccacgagtgactactgcggaaatggatgccagagccagtgtggtggcactgctaccactccgccgccatctcctccttctccaccaccgccagccactccttcccctccgtccccgccttctcctgttggtggagatgttagctctatcattacccgagaaatctttgaagagatgctcctgcatcgaaataacgccgcttgccctgcccgcggattctacacctacgaggcattcatcaccgccgctcgcttcttcagcggctttggcaccactggtgatttcaatacccgcaagagagaactagcagctttcttgggccagacctcccatgaaaccaccggttagttcatgctcaccgacaagaaaataaggggcgattatatggcatgcaccaacttatcaaatttgactttagcagaagtaccaatgagtgttgaaacaacttagtggtgattatatggatgaaatgttttattttaaaattgtttgattccgaaaattctacataggaacaagacttatatacagtagaataatatttttgatttgaaatgatgttttattagaaataaaatgaaaatgcgtaggagggtgggccaccgcacccgacggtccatatgcatggggatactgtttcaaggaggaggtcggccagcctggttcttattgtgttccctctacacagtggccatgcgccgctggtaaaagttactatggtcggggacccattcagctatcctagtaagtctcattctctttttcttattgttgattaattattaatattgaaaacgtaaaacattctctttttcttattgttgattaattattaatattgaaaacgtaaaaaataaacccaaaataaaagaataaaaaataaggatcagttttaatttttcttaacatcaaattttttgaaaaataaatttaaaattagagtaaaaaaattgaaattgaaggtagtcttaatattttttaactgcgggctgcttggtatttgactttgaaagcaactacaactacgggccgtccggtcaagccatcggacagccgctactggagaatccagatttggtagccggcgacgtgatcgtatcattcgaaacggctatatggttctggatgacgccgcagtacgacaagccgtcgtgccatgacgtaatgatcggaaaatggactccgtcagctcctgacattgcagccgggaggttcccaggttacggcgtgacgacgaacataatcaacggggggctcgagtgtgggagaggccctgatgcgagggtggctagtcgtattgggttctacgagaggtactgcgacattcttggcgtcgactacggagataacttggactgctacacccagtggccatttggtggatga

SEQ ID NO: 2 - DNA, 1673 bp, Nepenthes kassiana
atggagatagcatcagcaaaaatattctttggtttatccctcttgggactactagcgctgggatcagcggaacaatgcgggagtcaagctgggggagccgtgtgtccagggggcctgtgctgcagccaatatggctggtgtggcaccaccgacgactactgcggcgccggatgccagagccagtgcagctccagcggtggcgaccccagcagccttgttactagagacaagttcaatcagatgctcaagcaccggaatgatggcggctgccccgctaaaggcttctacacctacgatgctttcatagctgccgcaaagtccttccccgcttttgcagccaccggcgacgccgccacccgcaaaagggaaatcgccgccttcctcgcccaaacttcccatgaaactaccggttagttcatcccttttagtatcccctgtttcttgttgttcaatctccgctaccagtaatactccatcaagttcactagatgtattagtacacacagtaactttaattaagtttgtaatcatcgattcgaatatcttttaagtcgtttttttaactgcactgctgtgtacgggctgtgaaaatcttatgtaaatttgtagggaaattgacgatttaagtataaatatgagtttgttatatttggccagggggttgggcaagcgcaccagatggaccgtatgcgtggggatattgctatctcagggagcaaggcaaccctggatcttactgcgttcagagtgcccagtggccgtgtgtcgccggcaagaaatactacggtcgcggtcccatccagatttcctagtaagctttcgcatagcaaactttttatttaaccacaaatcctacgtgatgaagcgttaccggtagtatttatttttatttttattttgttaatcctactattttttattataaaatactaggtaaaaataaattatatctgaaaatattgctgaaaatgactaacctgtgagttctgtctactatcagactaattgagatgctttattagtccccacgtcctaaagcttaccttgtcgctgcaccccattacaaatttagcaatcctcggcaccaacccaaccccggcggctgggttaaatattactttgtcgctacacccccggccaaactttaacaaccatcagtcgcttctcatattttattcccttcgacgatcatatagcctataaacatgttaccttaaaatcatgtcctacacacagcacatcacgtaacgtaatgttaaactcgtgcttttgttatcagcaacttcaactacggagcagcggggaaagctataggggtggaccttctaaacaacccggatctggtcgagaaagaccctgtcgtttcgttcaagaccgcaatctggttctggatgacgccccagtctcccaagccctcgtgccatgaagtcatcaccgggcggtggacgccctcggcagccgacaaatcggccgggagggtgcctggattcggtgtggtcacaaacatcatcaacggtggggtcgaatgcggccatggacaagatgccagagtggccgacagaattggattctacaagcggtactgtgatatacttggagtcggctatggcaacaatttggactgctacaatcagaggcctttcggtaatgggcttttgtgggccaccgagtag

SEQ ID NO: 3 - DNA, 954 bp, Nepenthes kassiana
atggagatagcatcagcaaaaatattctttggtttatccctgttgggagtactagcgctgggatcagcggaacaatgcgggagtcaagctgggggagccgcgtgtccagggggcctgtgctgcagccaatttggctggtgtggcaccaccgacgactattgcgaagccggatgccagagccagtgcagctccagcggtggcgaccccagcagccttgttactagagacaagttcaatcagatgctcaagcaccggaatgatggcggctgccccgctaaaggcttctacacctacgatgctttcatagctgccgcaaagtccttccccgcttttgcagccaccggcgacgccgccacccgcaaaagggaaatcgccgccttcctcgcccaaacttcccatgaaactaccgggggttgggcaagcgcaccagatggaccgtatgcgtggggatattgctatctcagggagcaaggcaaccctggatcttactgcgttcagagtgcccagtggccgtgtgtcgccggcaagaaatactacggtcgcggtcccatccagatttcctacaacttcaactacggagcagcggggaaagctataggggtggaccttctaaacaacccggatctggtcgagaaagaccctgtcgtttcgttcaagaccgcaatctggttctggatgacgccccagtctcccaagccctcgtgccatgaagtcatcaccgggcggtggacgccctcggcagccgacaaatcggccgggagggtgcctggattcggtgtggtcacaaacatcatcaacggtggggtcgaatgcggccatggacaagatgccagagtggccgacagaattggattctacaagcggtactgtgatatacttggagtcggctatggcaacaatttggactgctacaatcagaggcctttcggtaatgggcttttgtgggccaccgag

SEQ ID NO: 4 - DNA, 954 bp, Nepenthes kassiana
atggagatagcatcagcaaaaatattctttggtttatccctcttgggactactagcgctgggatcagcggaacaatgcgggagtcaagctgggggagccgtgtgtccagggggcctgtgctgcagccaatatggctggtgtggcaccaccgacgactactgcggcgccggatgccagagccagtgcagcttcagcggtggcgaccccagcagccttgttactagagacaagttcaatcagatgctcaagcaccggaatgatggcggctgccctgctaaaggcttctacacctacgatgctttcatagctgccgcaaagtccttccccgcttttgcagccaccggcgacgccgccactcgcaaaagggaaatcgccgctttcctcgcccaaacttcccatgaaactaccgggggttgggcaagcgcaccagatggaccgtatgcgtggggatattgctatctgagggagcaaggcaaccctggatcttactgcgttcagagtgcccagtggccgtgtgttgccggcaagaaatactatggtcgcggtcccatccagatttcctacaacttcaactacggagcagcagggaaagctattggggtggaccttctaaacaacccggatctggtcgagaaagaccctgtcgtttcgttcaagaccgcaatttggttctggatgacgccccagtctcccaagccctcgtgccatgcagtcatcaccgggcggtggacgccctcggcagccgacaaatcagccgggagggtgcctggattcggtgtggtcacaaacatcatcaacggtggggtcgaatgcggccatggacaagatgccagagtggccgacagaattggattctacaagcggtactgtgatatacttggagtcggctatggcaacaatttggactgctacaatcagaggcctttcggtaatgggcttttgtgggccaccgag

SEQ ID NO: 5 - protein, 351 aa, Nepenthes kassiana
MNAPCFCFHAQKMRNHKHSTMRGWVVLLLLNLPFLSAFQCGQQAGGALCHSGLCCSQWGWCGTTSDYCGNGCQSQCGGTATTPPPSPPSPPPPATPSPPSPPSPVGGDVSSIITREIFEEMLLHRNNAACPARGFYTYEAFITAARFFSGFGTTGDFNTRKRELAAFLGQTSHETTGGWATAPDGPYAWGYCFKEEVGQPGSYCVPSTQWPCAAGKSYYGRGPIQLSYNYNYGPSGQAIGQPLLENPDLVAGDVIVSFETAIWFWMTPQYDKPSCHDVMIGKWTPSAPDIAAGRFPGYGVTTNIINGGLECGRGPDARVASRIGFYERYCDILGVDYGDNLDCYTQWPFGG

SEQ ID NO: 6 - protein, 318 aa, Nepenthes kassiana
MEIASAKIFFGLSLLGLLALGSAEQCGSQAGGAVCPGGLCCSQYGWCGTTDDYCGAGCQSQCSSSGGDPSSLVTRDKFNQMLKHRNDGGCPAKGFYTYDAFIAAAKSFPAFAATGDAATRKREIAAFLAQTSHETTGGWASAPDGPYAWGYCYLREQGNPGSYCVQSAQWPCVAGKKYYGRGPIQISYNFNYGAAGKAIGVDLLNNPDLVEKDPVVSFKTAIWFWMTPQSPKPSCHEVITGRWTPSAADKSAGRVPGFGVVTNIINGGVECGHGQDARVADRIGFYKRYCDILGVGYGNNLDCYNQRPFGNGLLWATE

SEQ ID NO: 7 - protein, 132 aa, Drosera capensis
GGWPTAPDGPYAWGYCFKQEQGNPGDYCVQSSTYPCAPGKKYYGRGPIQISNYNYGQCGAAINQPLLSNPDLVASNADVSFETAIWFWMTPQGSKPSCHAVATGQWTPSAADQAAGRVPGYGVITNIINGGL

SEQ ID NO: 8 - protein, 78 aa, Dionaea muscipula
NCNYGQCGESIGQPLLANPDLVANDVLISFETAIWFWMTPQWNKPSSHDVITGNWSPSSADQAAGRLPGYGVITNIIN

SEQ ID NO: 9 - DNA, 29 bp, Artificial sequence
  Single strand DNA primer
gctgacagggnaaagaatctttctatcac

SEQ ID NO: 10 - DNA, 29 bp, Artificial sequence
  Single strand DNA primer
gctgagatngttagcnccagaancctctg

SEQ ID NO: 11 - DNA, 28 bp, Artificial sequence
  Single strand DNA primer
ttnggncaagacntagcncactgagaac

SEQ ID NO: 12 - DNA, 30 bp, Artificial sequence
  Single strand DNA primer
gagtnccnccagttnatnatagtttacggt

SEQ ID NO: 13 - DNA, 26 bp, Artificial sequence
  Single strand DNA primer
ggggncaagaatcggnaatcgaaggg

SEQ ID NO: 14 - DNA, 28 bp, Artificial sequence
  Single strand DNA primer
canggnggagttagttagtaagaatctg

SEQ ID NO: 15 - protein, 8 aa, Artificial sequence
  Recombinant partial sequence of chitinase gene product
CEGKNFYT

SEQ ID NO: 16 - protein, 9 aa, Artificial sequence
  Recombinant partial sequence of chitinase gene product
QGFGATTIR

SEQ ID NO: 17 - protein, 10 aa, Artificial sequence
  Recombinant partial sequence of chitinase gene product
FLGAQTSHET

SEQ ID NO: 18 - protein, 9 aa, Artificial sequence
  Recombinant partial sequence of chitinase gene product
TNIINGGIL

SEQ ID NO: 19 - protein, 8 aa, Artificial sequence
  Recombinant partial sequence of chitinase gene product
WGQNGNEG

SEQ ID NO: 20 - protein, 9 aa, Artificial sequence
  Recombinant partial sequence of chitinase gene product
VQFYNNPPC

SEQ ID NO: 21 - DNA, 31 bp, Artificial sequence
  Single strand DNA primer
gaaaatggactccgtcagatcctgacattgc

SEQ ID NO: 22 - DNA, 31 bp, Artificial sequence
  Single strand DNA primer
gccccttattttcttgtcggtgagcatgaac

SEQ ID NO: 23 - DNA, 34 bp, Artificial sequence
  Single strand DNA primer
cgtttcgttcaagaccgcaatctggttctggatg

SEQ ID NO: 24 - DNA, 35 bp, Artificial sequence
  Single strand DNA primer
ctagtgaacttgatggagtattactggtagcggag

SEQ ID NO: 25 - DNA, 34 bp, Artificial sequence
  Single strand DNA primer
ctacaatcagaggcctttcggtaatgggcttttg

SEQ ID NO: 26 - DNA, 35 bp, Artificial sequence
  Single strand DNA primer
catcattccggtgcttgagcatctgattgaacttg

SEQ ID NO: 27 - DNA, 34 bp, Artificial sequence
  Single strand DNA primer
cgacggtccatatgcatggggatactgtttcaag

SEQ ID NO: 28 - DNA, 34 bp, Artificial sequence
  Single strand DNA primer
caaatggccactgggtgtagcagtccaagttatc

SEQ ID NO: 29 - DNA, 22 bp, Artificial sequence
  Single strand DNA primer
cgtggggatattgctatctcag

SEQ ID NO: 30 - DNA, 20 bp, Artificial sequence
  Single strand DNA primer
ctactcggtggcccacaaaa

SEQ ID NO: 31 - DNA, 23 bp, Artificial sequence
  Single strand DNA primer
gtaaaactggaccagacgtagtc

SEQ ID NO: 32 - DNA, 22 bp, Artificial sequence
  Single strand DNA primer
cgggaatgaaggaaccttcaac

SEQ ID NO: 33 - DNA, 23 bp, Artificial sequence
  Single strand DNA primer
gttgggtagtgcttctgctgctc

SEQ ID NO: 34 - DNA, 26 bp, Artificial sequence
  Single strand DNA primer
catatcatcatccaccaaatggccac

SEQ ID NO: 35 - DNA, 29 bp, Artificial sequence
  Single strand DNA primer
catcataacgaaaatggagatagcatcag

SEQ ID NO: 36 - DNA, 22 bp, Artificial sequence
  Single strand DNA primer
cggttattgggcctactcggtg

SEQ ID NO: 37 - DNA, 33 bp, Artificial sequence
  Single strand DNA primer
gaagcttccatgaatgctccgtgcttctgcttc

SEQ ID NO: 38 - DNA, 31 bp, Artificial sequence
  Single strand DNA primer
gcaagcttgtccaccaaatggccactgggtg

SEQ ID NO: 39 - DNA, 36 bp, Artificial sequence
  Single strand DNA primer
gagatagcatcagcaaaaatattctttggtttatcc

SEQ ID NO: 40 - DNA, 28 bp, Artificial sequence
  Single strand DNA primer
gcctcggtggcccacaaaagcccattac

SEQ ID NO: 41 - DNA, 31 bp, Artificial sequence; identical to SEQ ID NO: 21
  Single strand DNA primer

SEQ ID NO: 42 - DNA, 31 bp, Artificial sequence; identical to SEQ ID NO: 22
  Single strand DNA primer

SEQ ID NO: 43 - DNA, 34 bp, Artificial sequence; identical to SEQ ID NO: 23
  Single strand DNA primer

SEQ ID NO: 44 - DNA, 35 bp, Artificial sequence; identical to SEQ ID NO: 24
  Single strand DNA primer

SEQ ID NO: 45 - DNA, 34 bp, Artificial sequence; identical to SEQ ID NO: 25
  Single strand DNA primer

SEQ ID NO: 46 - DNA, 35 bp, Artificial sequence; identical to SEQ ID NO: 26
  Single strand DNA primer

SEQ ID NO: 47 - protein, 37 aa, Nepenthes kassiana
  misc_feature (1)..(37): Ch1 partial sequence signal peptide
MNAPCFCFHAQKMRNHKHSTMRGWVVLLLLNLPFLSA

SEQ ID NO: 48 - DNA, 1717 bp, Nepenthes kassiana
  misc_feature (1189)..(1189): Any nucleotide
atgaatgctccgtgcttctgcttccatgcacaaaaaatgcgaaaccacaagcacagtaccatgaggggttgggtagtgcttctgctgctcaatttacctttcctttcagcattccaatgcggccaacaagccggtggagcgctgtgccacagtggactctgctgcagccagtggggttggtgtggtaccacgagtgactactgcggaaatggatgccagagccagtgtggtggcactgctaccactccgccgccatctcctccttctccaccaccgccagccactccttcccctccgtccccgccttctcctgttggtggagatgttagctctatcattacccgagaaatctttgaagagatgctcctgcatcgaaataacgccgcttgccctgcccgcggattctacacctacgaggcattcatcaccgccgctcgcttcttcagcggctttggcaccactggtgatttcaatacccgcaagagagaactagcagctttcttgggccagacctcccatgaaaccaccggttagttcatgctcaccgacaagaaaataaggggcaccatgacgtaaatccacacagcaaccgtataatgccgtataataactatcggatcattattcaatgtcatatgctaattcttttattttaattaaaaaaatatttttaattagttttttaataacaaaaaatagtggtattggataataattctatgacaaccgtatgtacttatacggatgtcatacaaatctgcatcgcagcgttcctgttttacactgatatgcaccaacttaccaaatttgactttaacagaagtacaaatgactgttgacactacatagtatattttattttaaaattgtttgattctgaaaattctacataggaacaagaattatattaagtagaacaatatttttgatttgaaatgatgttttattagaaattaaatgaaaatgcgtaggagggtgggccaccgcacccgacggtccatatgcatggggatactgtttcaaggaggaggtcggccagcctggttcttattgtgttccctctacacagtggccatgcgccgctggtaaaagttactatggtcgaggacccattcagctatcctagtaagtctcattcacttttccttattgttgaataattattaatatcgaaaacgcgaaaaataancccaaaataaaagaataaaaaatagggatcaattttaatttttcttaacaacaaattttttgaaaaataaatttaaaattaaagtaaaaaaattgaaattgaanatagttttaatatttnttaactgngggctgnttggtatttgactttgaaagcaactacaactacgggccgtccggtcaagccatcggacagccgctactggagaatccagatttggtagccggcgacgtgatcgtatcattcgaaacggctatatggttctggatgacgccgcagtacgacaagccgtcgtgccatgacgtaatgatcggaaaatggactccgtcagctcctgacattgcagccgggaggttcccaggttacggcgtgacgacgaacataatcaacggggggctcgagtgtgggagaggccctgatgcgagggtggctagtcgtattgggttctacgagaggtactgcgacattcttggcgtcgactacggagataacttggactgctacacccagtggccatttggtggatga

SEQ ID NO: 49 - protein, 28 aa, Nepenthes kassiana
  misc_feature (1)..(28): Ch1 partial sequence Proline rich domain
GGTATTPPPSPPSPPPPATPSPPSPPSP

(19) United States
(12) Patent Application Publication
(10) Pub. No.: US 2004/0078842 A1
(43) Pub. Date: Apr. 22, 2004

(54) Chitinases, derived from carnivorous plants polynucleotide sequences encoding thereof, and methods of isolating and using same

(76) Inventors: Aviah Zilberstein, Holon (IL); Haviva Eilenberg, Ramat Hasharon (IL); Silvia Schuster, Raanana (IL)

Correspondence Address: Anthony Castorina, G E Ehrlich, Suite 207, 2001 Jefferson Davis Highway, Arlington, VA, 22202, US

(21) Appl. No.: 10/451,794

(22) PCT Filed: Jan. 17, 2002
(86) PCT No.: PCT/IL02/00044

(51) Int. Cl. A01H 1/00; C12N 9/24; A61K 38/47; C12N 15/82

(52) U.S. Cl. 800/279; 424/94.61; 435/200

(57) ABSTRACT

The present invention provides an enzymatic composition comprising at least one protein isolated from a tissue or soup of a carnivorous plant, the at least one protein being characterized with an endo-chitinase activity.

### FIELD AND BACKGROUND OF THE INVENTION

The present invention is of chitinases derived from carnivorous plants polynucleotide sequences encoding such chitinases, and methods of isolating and using such chitinases to reduce susceptibility of plants to chitin-containing pathogens to render plants refractory to chilling and frost conditions and to treat individuals suffering from diseases or conditions associated with a chitin-containing pathogen, such as Candida albicans.

Plant pathogens affect the overall crop production and may often cause total destruction of a crop. A range of cellular processes enables plants to resist pathogen infection and prevent the development of associated disease symptoms. These responses include among others the de novo synthesis of a set of protein families known as Pathogen-Related proteins (PR proteins). However, the use of natural plant products for protection against plant pathogens often entails enhancement of existing metabolic pathways to increase synthesis of products involved in plant defense mechanism. Such metabolic alteration may have adverse effects on normal development, production of assimilates, ability to express yield and quality capacities and others. Consequently, the modification of plant defense systems by transgenic expression of more potent PR proteins from heterologous sources is of major importance (see, for example, European Patent Application 0 392 225).

One of the best characterized PR proteins is chitinase (E.C.3.2.2.14) which catalyzes the hydrolysis of chitin, a 1,4 linked polymer of N-acetyl-D-glucosamine (NAG) which is a major cell wall component of most filamentous fungi with the exception of the Oomycetes. It is also an important component of arthropods, nematodes and mollusks. In fungi, chitinases hydrolyze chitin at the tip of the growing mycelium, inhibit sporulation and hydrolyze the cell wall of haustoria (Carr and Klessing, 1989) This hydrolytic activity plays a direct role in slowing fungal growth and delaying or preventing the invasion of pathogens into plant tissues. Chitinase also plays an indirect but important role in releasing breakdown products from fungal cell walls, which then act as signal molecules in eliciting the plant defense response (Graham and Sticklen, 1994).

Most of the plant chitinases isolated to date are endo-chitinases which release small polymers of the core chitin structure. The molecular weight range of these enzymes is between 25-40 kDa, they are usually active as monomers with acidic optima (pH 3-6.5), appear to require no cofactors and are stable at a wide range of temperatures. Chitinases have been isolated from many plant species and they are classified into 5 classes (I-V) according to their multi-domain structure (Collinge et al., 1993; Hamel et al., 1997).

Class I chitinases are mainly composed of basic proteins (with basic pI values), mostly targeted to the vacuoles and found in both monocots and dicots. These enzymes display high specific activities and are responsible for the majority of the plant chitinolytic activity in roots, shoots and flowers (Legrand et al., 1987). Class I chitinases are composed of five structural domains: (i) N-terminal signal peptide (20-27 amino acids residues) that routes the protein into the endoplasmic reticulum; (ii) cysteine rich domain (CRD of ˜40 amino acids), which is involved in chitin binding and contains eight cysteine residues in highly conserved positions; (iii) proline (mostly hydroxyproline)-rich hinge region (HR) that varies in size; (iv) catalytic domain (CD>220 amino acids), comprising the central domain of the protein that shows high homology to the catalytic domain of class II and IV chitinases and low homology to the CD of bacterial chitinases; and (v) carboxy-terminal extension (CTE), which targets the protein into the vacuole and is present in most of class I chitinases (Graham and Sticklen, 1994; Hamel et al., 1987). Rapid release of large amounts of the vacuole-compartmentalized chitinase occurs during cell lysis resulting from hypersensitive response to pathogen invasion. Several class I-basic chitinases, which are devoid of CTE, have also been characterized. These chitinases are secreted to the extracellular space (Legrand et al., 1987; Swegle et al., 1992; Vad et al., 1991).

Class II chitinases are acidic (with acidic pI), containing only the signal peptide and catalytic domain. The latter shows a high amino acid sequence homology to the catalytic region of class I and class IV chitinases. The specific activity of acidic chitinases is lower than that of class I-chitinases. It is assumed that the primary function of class II-chitinases is to generate elicitors of defense responses by partial degradation of the fungal pathogen cell wall (Graham and Sticklen, 1994)

Class III chitinases include basic or acidic extracellular proteins with chitinase/lysozyme activity. Their catalytic domain is different from that of class I and II but shares significant identity with chitinases from yeast and filamentous fungi.

Class IV chitinases share structural domain similarity with class I chitinases but not a high amino acid sequence identity. All of class IV enzymes lack the CTE and are therefore targeted to the apoplast. In addition, amino acid sequence alignment with class I proteins showed four distinct deletions; one in the chitin binding domain and three within the catalytic domain. This group include the PR4 chitinase from bean, the ChB4 from Canola and many others (Hamel et al., 1997)

Class V chitinases share some homology to exo-chitinases of bacterial origins, e.g. Serracia marcescens, Bacillus circulans and Streptomyces plicatus.

Several plant chitinases whose structure differs from the above mentioned categories have also been characterized. Several acidic chitinases appear to have the structural composition of class I (Van Damme et al., 1993). Another unusual enzyme is the agglutinin type of chitinase from Urtica dioica (UDA), comprising two N-terminal chitin binding domains and a catalytic domain with amino acid sequence homology to class I chitinases. This enzyme, instead of cleaving chitin polymers, displays ability to cross-link chitin chains at the tips of the invading fungal mycelia and thereby inhibiting pathogen development (Lerner and Raikhel, 1992). A homodimeric holoenzyme, possessing both endo-chitinase and insect alpha-amylase inhibition activity, was isolated from seeds of Job's tear (Croix lachrymosa) (Ary et al., 1989). Thus, extraordinary proteins with chitinase activity have evolved in diverse plant systems. However despite the abundance of data regarding plant chitinases, to date no chitinases have been isolated from carnivorous plants.

Plant defense systems against pathogens of important crops may be modified by introduction of foreign genes encoding proteins having a wide spectrum of anti-fungal activities. Methods for producing transgenic plants among the monocotyledenous plants are well documented. Successful transformation and plant regeneration have been achieved in asparagus (Asparagus officinalis; Bytebier et al. (1987); barley (Hordeum vulgare; Wan and Lemaux (1994)); maize (Zea mays; Rhodes et al. (1988)); Gordon-Kamm et al. (1990); Fromm et al. (1990); Koziel et al. (1993); oats (Avena sativa; Somers et al. (1992)); orchardgrass (Dactylis glomerata; Horn et al. (1988)); rice (Oryza sativa, including indica and japonica varieties; Tornyama et al. (1988)); Zhang et al. (1988); Luo and Wu (1988); Zhang and Wu (1988); Christou et al. (1991); rye (Secale cereale; De la Pena et al. (1987)); sorghum (Sorghum bicolor; Cassas et al. (1993)); sugar cane (Saccharum spp.; Bower and Birch (1992)); tall fescue (Festuca arundinacea; Wang et al. (1992)); tuifgrass (Agrostis palustris; Zhong et al. (1993)); wheat (Triticum aestivum; Vasil et al. (1992); Troy Weeks et al. (1993); Becker et al. (1994)).

Plant genes encoding cell wall degrading enzymes, especially chitinases, have been used to enhance plant resistance to fungal pathogens (see, for example, U.S. Pat. Nos. 6,291,647; 6,280,722 to Melchers, et al and Moar, respectively), but no single genes have produced an adequate level of resistance (Broglie et al., 1991; Punja and Raharjo, 1996; Zhu et al., 1994; Jach et al., 1995). Furthermore, although fungal chitinases derived from Trichoderma harzianum have been reported effective on pathogens in tobacco, potato (Lorito et al., 1998) and apple (Bolar et al., 2000), persistent sensitivity to multiple pathogens remains a common and costly problem in crops incorporating antifungal genes. Recently, a broad spectrum antifungal from alfalfa for use in transgenic fungal resistant crops was disclosed by Liang et al (U.S. Pat. No. 6,329,504), however, no biochemical characterization of the antifungal activity was provided.

The specific activity of plant pathogen resistance proteins is a critical consideration in the choice of genes and their products for protection against disease. Thus, it would be advantageous to have novel plant pathogen resistant proteins of high specific activity.

Prior art describes various applications of the enzymatic digestion of chitin by chitinases in the treatment and prevention of plant and animal disease. For example, Jaynes, et al disclosed the use of non-plant antimicrobial proteins to confer disease resistance in transgenic animals (U.S. Pat. No. 6,303,568), among them, chitinase. A novel chitinase from B. thuringensis was also reported by Moar (U.S. Pat. No. 6,280,722). However, no mention of chitinases from carnivorous plants has been made. Furthermore, the application of plant chitinases for human pathogens has not been reported.

Aside from being deleterious to plants, chitin containing organisms, such as fungi, protozoa and worms (helminth) are also the causative agent in a variety of infectious diseases in humans and animals.

The limited number of presently available anti-fungal drugs are in general not very potent. Fungal infections are regularly encountered in immuno-incompetent people, currently most frequently in patients with acquired immunodeficiency syndrome (AIDS). Most fungal infections of the skin are treated with topical preparations. Visceral infections and cuticular infections require prolonged systemic therapy.

The most frequent fungal infection is caused by Candida albicans. The organism is a common commensal of the oral and vaginal mucosae but can become a pathogen on damaged skin, in severely ill patients, in patients who have specific immune deficiency, and in patients receiving broad-spectrum antibiotics when the local microbial ecology is disturbed. Extreme consequences of Candida infection can be pneumonia, endocarditis, septicaemia and even death. The only effective treatment is intravenous administration of amphotericin B. Administration of this drug can result in serious adverse effects that are accompanied by hypotension and collapse. For that reason an initial test dose is infused to determine the tolerance. Flucytosine is a synthetic fluorinated pyrimidine which enters fungal cells and inhibits metabolism by interfering with DNA and RNA synthesis. The compound is usually given in combination with amphotericin B for treatment of systemic fungal infections. When administered alone, resistance towards flucytosine rapidly develops.

Other species of fungi that can cause severe infectious diseases in man are Aspergillus, Cryptococcus, Coccidioides, Paracoccidioides, Blastomyces, Sporothrix, and Histoplasma capsulatum.

Thus, there is a continuing need to identify and characterize novel pathogen protective compounds, particularly those that would be effective against plant and human pathogenic fungi, which may be expressed in transgenic organisms in amounts sufficient to provide protection against the pathogen(s).

### SUMMARY OF THE INVENTION

According to one aspect of the present invention there is provided an enzymatic composition comprising at least one protein isolated from a tissue or soup of a carnivorous plant, the at least one protein being characterized with an endo-chitinase activity.

According to another aspect of the present invention there is provided an enzymatic composition comprising a protein extract of a tissue or soup of a carnivorous plant, wherein the protein extract includes at least one protein exhibiting endo-chitinase activity.

According to further features in preferred embodiments of the invention described below the at least one protein is characterized by a pI below 10.

According to still further features in the described preferred embodiments the at least one protein is not reactive with an anti ChiAII polyclonal antibody.

According to still further features in the described preferred embodiments the at least one protein does not exhibit endo-chitinase activity following exposure to reducing conditions.

According to still further features in the described preferred embodiments the at least one protein is characterized by an apparent molecular weight of about 32.7 kDa as determined by 12% SDS-PAGE.

According to still further features in the described preferred embodiments the at least one protein is characterized by an apparent molecular weight of about 36 kDa as determined by 12% SDS-PAGE.

According to still further features in the described preferred embodiments there is provided a pharmaceutical composition comprising as an active ingredient the enzymatic composition and a pharmaceutically acceptable carrier or diluent.

According to still further features in the described preferred embodiments the at least one protein is characterized by an anti-fungal activity.

According to still further features in the described preferred embodiments the anti-fungal activity is fungicidal activity.

According to still further features in the described preferred embodiments the anti-fungal activity is anti Candida albicans activity.

According to still further features in the described preferred embodiments there is provided composition for disinfesting chitin-containing pathogens, the composition comprising as an active ingredient the enzymatic composition and a carrier or diluent.

According to still further features in the described preferred embodiments there is provided an agronomical composition comprising as an active ingredient the enzymatic composition and an agronomically acceptable carrier.

According to still further features in the described preferred embodiments the at least one protein is at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

According to still further features in the described preferred embodiments the at least one protein is as set forth in SEQ ID NOs: 5, 6, 7 or 8 or active portions thereof.

According to still further features in the described preferred embodiments the tissue is trap tissue and/or leaf tissue.

According to still further features in the described preferred embodiments the soup is trap soup.

According to still further features in the described preferred the carnivorous plant is selected from the group consisting of Nepenthes ssp., Drosera sp., Dionea sp. and Sarracenia sp.

According to yet another aspect of the present invention there is provided an isolated nucleic acid comprising a polynucleotide sequence encoding a polypeptide having an endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

According to still further features in the described preferred embodiments the polynucleotide sequence is selected from the group consisting of SEQ ID NOs: 1, 2, 3, 4 and 48 or active portions thereof.

According to still further features in the described preferred embodiments the polypeptide is selected from the group consisting of SEQ ID NOs: 5, 6, 7 and 8 or active portions thereof.

According to still further features in the described preferred embodiments the polynucleotide sequence is selected from the group consisting of a genomic polynucleotide sequence, a complementary polynucleotide sequence and a composite polynucleotide sequence.

According to still further features in the described preferred embodiments there is provided a nucleic acid construct comprising the isolated nucleic acid.

According to still further features in the described preferred embodiments there is provided a host cell comprising the nucleic acid construct.

According to still another aspect of the present invention there is provided an isolated nucleic acid comprising a polynucleotide sequence encoding a polypeptide having an endo-chitinase activity and including a signal peptide of at least 30 amino acids.

According to still further features in the described preferred embodiments the signal peptide is for protein secretion.

According to still further features in the described preferred embodiments the polynucleotide sequence is set forth in SEQ ID NOs: 1 or 48 or active portions thereof.

According to still further features in the described preferred embodiments the polypeptide is set forth in SEQ ID NO: 5 or active portions thereof.

According to still further features in the described preferred embodiments the signal peptide is set forth in SEQ ID NO: 47.

According to an additional aspect of the present invention there is provided an isolated nucleic acid comprising at least 67% identical with SEQ ID NO: 1 or at least 75% identical with SEQ ID NO: 2 as determined using the BestFit software of the Wisconsin sequence analysis package, utilizing the Smith and Waterman algorithm, where gap weight equals 50, length weight equals 3, average match equals 10 and average mismatch equals −9.

According to still further features in the described preferred embodiments the polynucleotide sequence is selected from the group consisting of SEQ ID NOs: 1, 2, 3, 4 and 48 or active portions thereof.

According to still further features in the described preferred embodiments the polynucleotide sequence is selected from the group consisting of a genomic polynucleotide sequence, a complementary polynucleotide sequence and a composite polynucleotide sequence.

According to yet an additional aspect of the present invention there is provided an oligonucleotide of at least 17 bases specifically hybridizable with an isolated nucleic acid set forth in SEQ ID NO: 1, 2, 3, 4 or 48.

According to still an additional aspect of the present invention there is provided a pair of oligonucleotides each of at least 17 bases specifically hybridizable with SEQ ID NO: 1, 2, 3, 4 or 48 in an opposite orientation so as to direct specific amplification of a portion thereof in a nucleic acid amplification reaction.

According to a further aspect of the present invention there is provided an isolated polypeptide having endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

According to yet a further aspect of the present invention there is provided an isolated polypeptide selected from the group consisting of SEQ ID NOs: 5, 6, 7 and 8 or active portions thereof.

According to still a further aspect of the present invention there is provided a method of treating an individual having a disease or a condition associated with a chitin-containing pathogen, the method comprising administering to the individual a therapeutically effective amount of a pharmaceutical composition including as an active ingredient a protein extract derived from a trap soup or a trap tissue of a carnivorous plant, the protein extract including at least one protein exhibiting endo-chitinase activity.

According to still a further aspect of the present invention there is provided a method of generating a pharmaceutical composition useful for treating a disease or a condition associated with a chitin-containing pathogen, the method comprising: (a) extracting a protein fraction from a trap soup or a trap tissue of a carnivorous plant, the protein fraction exhibiting endo-chitinase activity; and (b) mixing the protein fraction with a pharmaceutically acceptable carrier or diluent, thereby generating the pharmaceutical composition useful for treating the disease or the condition associated with the chitin-containing pathogen.

According to still a further aspect of the present invention there is provided a method of reducing susceptibility of a plant to a chitin-containing pathogen, the method comprising expressing within the plant an exogenous polypeptide having an endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

According to still a further aspect of the present invention there is provided a method of isolating polypeptides exhibiting a high endo-chitinase activity, the method comprising: (a) preparing a protein extract from a trap tissue or a trap soup of a carnivorous plant; and (b) isolating from the protein extract a chitinase active fraction, thereby isolating polypeptides exhibiting high endo-chitinase activity.

According to still further features the method further comprising exposing the trap tissue or the trap soup to chitin prior to (a).

According to still a further aspect of the present invention there is provided a method of reducing susceptibility of a plant to cold damage, the method comprising, exposing a plurality of plants to a composition including as an active ingredient a protein extract derived from a soup or tissue of a carnivorous plant, the protein extract including at least one protein exhibiting endo-chitinase activity.

According to still a further aspect of the present invention there is provided a plant, a plant tissue or a plant seed comprising an exogenous polynucleotide sequence encoding a polypeptide having an endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

According to still a further aspect of the present invention there is provided an isolated nucleic acid comprising a polynucleotide sequence encoding a polypeptide having an endo-chitinase activity and including a proline rich region having at least 10 and no more than 15 proline amino acids.

According to still further features in the described preferred embodiments wherein the proline rich region includes 6 putative glycosylation sites.

According to still further features in the described preferred embodiments wherein the polynucleotide sequence is set forth in SEQ ID NOs: 1 or 48 or active portions thereof.

The present invention successfully addresses the shortcomings of the presently known configurations by providing novel chitinases, derived from carnivorous plants polynucleotide sequences encoding such chitinases, and methods of isolating and using such chitinases to reduce susceptibility of plants to chitin-containing pathogens to render plants refractory to chilling and frost conditions and to treat individuals suffering from diseases or conditions associated with a chitin-containing pathogen, such as Candida albicans.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a chitinase activity gel demonstrating the presence of novel chitinase activity in Nepenthes trap soup. Leaf, open trap and closed trap tissue extracts (150 μl), and trap soup (75 μl) samples were separated on 15% native PAGE. After electrophoresis the gel was overlayed with a second gel containing 0.01% (w/v) glycol chitin, incubated overnight at 37° C., and stained 5 minutes with 0.01% Calcofluor white M2R. Chitinase activity (dark staining bands of lytic activity) was visualized by UV illumination. Note the presence of a novel band of chitinase activity in trap soup, migrating differently than that of the other plant tissues.

FIGS. 2a-b are Western blots demonstrating the absence of antigenic similarity between the novel trap soup chitinase and S. marcescens chitinase. Tissue extracts (leaf=L; trap tissue=C) and trap soup (S) from Nepenthes, and E. coli extract containing S. marcescens extract (Ser) were separated on 15% SDS-PAGE, blotted onto PVDF membrane and probed with rabbit polyclonal antibodies recognizing S. marcescens ChiAII chitinase. Immunoreactive bands were visualized by binding of Alkaline Phosphatase conjugated goat anti rabbit antibodies. FIG. 2a-samples contain 50 μl leaf (L) and trap tissue (C) extracts, 40 μl trap soup (S) and 100 ng extract of E. coli cells overexpressing+S. marcescens ChiAII chitinase (Ser). FIG. 2b—samples contain 50 μl leaf extract (L), 100 ng of E. coli cells overexpressing S. marcescens chitinase (Ser) and 875 μl concentrated trap soup (S). Arrowheads demonstrate immunoreactive leaf extract and E. coli bands, while even 22 fold concentration of trap soup revealed no antigenic cross reactivity with the antibodies against S. marcescens;

FIG. 3 is a semidenatured chitinase activity gel demonstrating the resistance of novel chitinase activity in Nepenthes trap soup to SDS denaturation. Aliquots of leaf (L) and trap tissue (C) extracts (150 μl), trap soup (S) (75 μg) and 0.6 μg extract of E. coli cells overexpressing S. marcescens ChiAII chitinase (Ser) were separated on 15% SDS PAGE without boiling or 2-mercaptoethanol. After electrophoresis the gel was renatured by incubation in 40 mM Tris-HCl, pH 8.8, 1% casein, 2 mM EDTA, then overlayed with a second gel containing 0.01% (w/v) glycol chitin, incubated overnight at 37° C., and stained 5 minutes with 0.01% Calcofluor white M2R. Chitinase activity (dark staining bands of lytic activity) was visualized by UV illumination. Note the consistently slower migration of the novel band of trap soup chitinase activity (S);

FIGS. 4a-b are denaturing chitinase activity gels demonstrating the sensitivity of novel Nepenthes trap soup chitinase activity to SDS and 2-mercaptoethanol denaturation. Aliquots of leaf (Leaf) extracts (150 μl), trap soup (Soup) (75 μl) and 0.6 μg extract of E. coli cells overexpressing S. marcescens ChiAII chitinase (Ser) were prepared in SDS and 2-mercaptoethanol, either without (FIG. 4a) or with (FIG. 4b) boiling for 5 minutes, and separated on 15% SDS PAGE. After electrophoresis the gel was renatured by incubation in 40 mM Tris-HCl, pH 8.8, 1% casein, 2 mM EDTA, then overlayed with a second gel containing 0.01% (w/v) glycol chitin, incubated overnight at 37° C., and stained 5 minutes with 0.01% Calcofluor white M2R. Chitinase activity (dark staining bands of lytic activity) was visualized by UV illumination. Note the absence of the novel band of trap soup chitinase activity (S), but not that of the other chitinases, following denaturation with or without boiling;

FIG. 5 is a Coomassie blue stained SDS PAGE demonstrating the high specific activity Nepenthes trap soup chitinase. Samples of concentrated trap soup (S)(600 μl), 4 μg protein extract of E. coli overexpressing the 58 kDa S. marcescens ChiAII chitinase (Ser) and size markers (SM) were separated on 15% SDS PAGE and visualized with Coomassie blue staining. Note the undetectable levels of protein in the trap soup.

FIG. 6 illustrates the purification and concentration of Nepenthes trap soup enzyme by FPLC. Trap soup was desalted and brought to pH 10 by gel filtration on Sephadex G-25, loaded onto a Mono Q anion exchange column and the bound chitinase was eluted with increasing concentrations of NaCl. Chitinase activity was determined on chitinase activity gels, as described in Methods, and protein concentration evaluated according to the absorbance at 280 nm. The vertical arrows denote fractions expressing chitinase activity;

FIG. 7 is a SDS-PAGE separation demonstrating purification of chitinase activity in FPLC fractions of closed Nepenthes trap soup. Trap soup was loaded onto an anion exchange column and the bound proteins were eluted with NaCl gradient (0-600 mM), as described in FIG. 6. Each fraction (lanes 5-20) was then tested for chitinase activity on activity gels. Chitinase activity is indicated by +. Protein content of the fractions was analyzed by SDS-PAGE and silver staining. The “pool” lane contained 50 μl of pooled and concentrated (8 fold) FPLC purified fractions exhibiting chitinase activity (fractions 9-17). SM=size markers;

FIG. 8 is a chitinase activity gel demonstrating induction of multiple forms of Nepenthes trap soup chitinase by chitin injection. Approximately 1 mg of colloidal chitin, pH 5.0, was injected into a closed trap. Samples containing uninduced soup (prior to injection) (lane 3, 30 μl), 20 hr (lane 4, 30 μl) and 5 day (lane 5, 30 μl) post induction trap soup, concentrated (8 fold) FPLC purified uninduced soup chitinase (lane 2, 15 μl) and 0.6 μg extract of E. coli cells overexpressing Serratia ChiAII (lane 1) were separated on native 15% PAGE gels. After electrophoresis the gel was overlayed with an additional gel containing 0.01% (w/v) glycol chitin and assayed for chitinase activity. Note the presence of additional bands of inducible chitinase activity (lanes 4 and 5);

FIG. 9 is a SDS-PAGE separation and silver staining of Nepenthes trap soup demonstrating chitin-induced protein bands. Approximately 1 mg of colloidal chitin, pH 5.0, was injected into a closed trap. Samples containing 100 μl of uninduced trap soup (lane 1), and trap soup 4 days (lane 2), 8 days (lane 3), 14 days (lane 4) after induction, or 50 μl noninduced pooled, 8 fold concentrated, FPLC cleaned soup (lane 5) were separated on 12% SDS-PAGE. Migration of protein bands was visualized with silver staining. Note the appearance of at least 4 additional chitin-induced protein bands (lanes 2, 3 and 4);

FIGS. 10a-c are growth inhibition assay plates illustrating the fungicidal activity of chitin-induced Nepenthes trap soup chitinase on plant and human pathogens. In FIG. 10a the minimal inhibitory concentration (MIC) value for inhibition of the human pathogen Candida albicans was determined in broth as detailed in the Methods section. Further assessment of yeast mortality (minimal fungicidal concentration, MFC) was carried out by re-plating 100 ml of the trap soup-exposed cells on trap soup-free solid medium (Sabuaruad) and counting the number of colonies following 48 hrs of incubation at 28° C. Note the near-total absence of C. albicans colonies with exposure to 1:4 dilution (left plate) compared to 1:8 dilution (right plate) of trap soup. FIG. 10b illustrates the fungicidal activity of chitin-induced Nepenthes trap soup chitinase on the plant pathogen Septoria tritici. Liquid cultures of Septoria tritici conidia (2.5×10⁴ conidia/ml, 100 μl) were incubated for six days at 19° C. with 100 μl of increasing dilutions (1-1/32) of Nepenthes chitin-induced trap soup (total protein concentration in the undiluted sample=3.1 mg/ml). Minimal inhibitory dilution was 1:2, determined spectrophotometrically according to OD₅₅₀. Samples (50 μl) were plated on trap soup-free malt agar plates and incubated at the same conditions for an additional 6 days. The dilutions are indicated beside each sample (1, ½, 1/16, 1/32). Control cultures were incubated without trap soup (H₂O). Note that no S. tritici conidia survived exposure to undiluted trap soup (1). FIG. 10c illustrates the fungicidal activity of chitin-induced Nepenthes trap soup chitinase on Rhizoctonia solani and Aspergillium spp. mycelium development. Samples (20 μl) of 5 fold concentrated trap soup were applied to plates containing log phase culture of either Rhizoctonia or Aspergillus. Note the inhibition area formed near the site of trap soup chitinase application (arrow).

FIG. 11 is the complete nucleotide sequence of the Nepenthes trap soup basic chitinase 1 gene Nkchit1b (SEQ ID NO:1). Introns are marked in green, and the first methionine and stop codons are marked in red.

FIGS. 12a-b illustrate nucleic acid and deduced amino acid sequences of Nepenthes trap soup basic chitinase 2 gene Nkchit2b. FIG. 12a is a comparison of the deduced amino acid sequences of Nepenthes chitinase 2 cDNAs. cDNA was synthesized by RT-PCR strategy on mRNA isolated from trap secretory tissue. Several chitinase 2 PCR clones were isolated by using chitinase 2 gene-specific primers. Two types of cDNA sequences, Nkchit2b-II (SEQ ID NO:3) and Nkchit2b-III (SEQ ID NO:4) were identified. Of the six amino acid mismatches, those marked in green are identical to the original Nkchit2b, encoded by a genomic clone, and isolated by inverse PCR. FIG. 12b is the complete nucleotide sequence of the Nepenthes trap soup basic chitinase 2 gene Nkchit2b (SEQ ID NO:2). Introns are marked in green, and the first methionine and stop codons are marked in red.

FIG. 13 is the amino acid sequence alignment (PRETTYBOX) and functional domains of NkCHIT1b (ch1)(SEQ ID NO:5) and NkCHIT2b (ch2)(SEQ ID NO:6), deduced according to the structure of basic chitinases in the databases. Functional domains are indicated in color: signal peptide—green, cysteine rich domain—orange, hypervariable proline rich region—light blue, catalytic domain—red and C-terminal extension—purple.

FIG. 14 is a multiple sequence alignment of the amino acid sequences of known monocot and dicot chitinases revealing closest homology to NkCHIT1b (SEQ ID NO:5). Known chitinases are indicated by their NCBI Accession numbers: s40414—Oryza sativa 1; s39979—Oryza sativa 2; x56063—Oryza sativa 3; oriza—Oryza sativa 4 (383024); t03614—Oryza sativa 5; jc2071—Secale cereale 1; secale—Secale cereale 2 (741317); s38670—Triticum aestivum; af000966—Poa pratensis; 137289—Oryza sativa 6; z78202—Persea Americana; p51613—Vitis vinifera; and ch1—NkCHIT1b. Predicted glycosylation sites in NkCHIT1b are denoted by violet dots. Common functionally significant individual amino acids are indicated in color: cysteine (yellow)—involved in disulfide bridges; threonine and glutamine (red)—maintenance of active site geometry; glutamic acid and asparagine (green)—important in catalysis; and tyrosine (blue)—important for substrate binding in the catalytic cleft.

FIG. 15 is a multiple sequence alignment of the amino acid sequences of known monocot and dicot chitinases revealing closest homology to NkCHIT2b (SEQ ID NO:6). Known chitinases are indicated by their NCBI Accession numbers: y10373—Medicago truncatula; t09687—Medicago sativa; p21226—Pisum sativum; aj012821—Cicer arietinum; p06215—Phaseolus vulgaris 1; p36361—Phaseolus vulgaris 2; s57482—Vigna unguiculata; bean—Psophocarpus tetragonolobus (BAB13369); x56063—Oryza sativa 1; oriza—Oriza sativa 2 (383024); t03614—Oriza sativa 3; ch2—NkCHIT2b; p51613—Vitis vinifera; and z78202—Persea americana. Two amino acids, valine and glutamic acid, unique in NkCHIT2b when compared to all the other chitinases closely homologous to NkCHIT2b, are marked with a brown and black arrow, respectively. A predicted glycosylation site in NkCHIT2b is denoted by a violet dot. Common functionally significant individual amino acids are indicated in color: cysteine (yellow)—involved in disulfide bridges; threonine and glutamine (red)—maintenance of active site geometry; glutamic acid and asparagine (green)—important in catalysis; and tyrosine (blue)—important for substrate binding in the catalytic cleft.

FIGS. 16a-b illustrate conserved amino acids in NkCHIT2b. FIG. 16a is a segment of a multiple sequence alignment of NkCHIT2b with the amino acid sequences of monocot and dicot chitinases from the gene bank closely homologous to NkCHIT2b. A segment of the amino acid sequence of barley (Hordeum vulgare L., p23951) endochitinase is included for comparison and prediction of three dimensional structure. Amino acids implicated in chitinase function are marked by an arrow. FIG. 16b depicts a three dimensional structural model (SWISS-MODEL Protein Modeling, http://www.expasy.ch/swissmod/SWISS-MODEL) of NkCHIT2b, viewed from the right and left of the molecule. Individual amino acids implicated in chitinase function are highlighted. Note the location of amino acids Glu 134, Glu 156, Asn 191 and Phe 190 within the catalytic cleft.

FIG. 17 depicts the predictions of possible O-glycosylation sites in the amino acid sequence of novel Nepenthes trap soup basic chitinase NkCHIT1b. Predictions were performed with the ExPaSy Molecular Server (NetOGlyc prediction, http://www.cbs.dtu.dk/services/NetOGlyc) which predicts post-translational modifications. Note that NkCHIT1b has nine possible glycosylation sites, concentrated in the proline rich hinge region.

FIG. 18 depicts the predictions of possible O-glycosylation sites in the amino acid sequence of novel Nepenthes trap soup basic chitinase NkCHIT2b. Predictions were performed with the ExPaSy Molecular Server (NetOGlyc prediction, http://www.cbs.dtu.dk/services/NetOGlyc) which predicts post-translational modifications. Note that NkCHIT2b has only one possible glycosylation site.

FIG. 19 depicts the results of RT-PCR analysis of mRNA from uninduced Nepenthes trap tissue, illustrating differential expression of novel chitinases. mRNA isolated from closed traps by hot borate/proteinase K method was used to synthesize cDNA with an oligo dT primer. Consecutive PCR amplifications used the specific primers indicated. A control reaction lacking reverse transcriptase (−RT) was included for each gene-specific PCR reaction. A reaction with genomic DNA as template was also included for positive control. PCR products were separated by acrylamide gel electrophoresis, stained with EtBr and visualized under UV illumination. Samples contained RT-PCR products using specific Nkchit1b primers (lane 1=+RT, lane 2=−RT control) and genomic Nkchit1b DNA as template (lane 3); specific Nkchit2b primers (lane 4=+RT, lane 5=−RT control), genomic Nkchit2b DNA as template (lane 6), 5′ and 3′ control primers (lanes 7 and 8, respectively); specific acidic chitinase primers (lane 9=+RT, lane 10=−RT control) and genomic acid chitinase DNA as template (lane 11); basic chitinase degenerate primer Bs2 (lane 12=+RT, lane 13=−RT control) and genomic basic chitinase DNA as template (lane 14); and basic chitinase degenerate primer Bs1 (lane 15=+RT, lane 16=−RT control) and genomic Bs1 DNA as template (lane 17). Note that Nkchit1b produces only very faint specific bands with Nkchit1b (lane 1) and acid chitinase (lane 9) primers, while a strong specific band is produced with the Nkchit2b primers (lane 4). Also note that the higher molecular weight products were produced when genomic DNA was used as template (lanes 3 and 6, for example). High and low molecular weight markers (SM) were also included.

FIG. 20 depicts the results of RT-PCR analysis of mRNA from chitin-induced Nepenthes trap tissue, illustrating specific induction of novel chitinases. mRNA was isolated from trap tissue four days after induction with chitin injection. Isolation of mRNA, cDNA synthesis, RT-PCR, separation and visualization of products, and identity of specific primers as described in FIG. 19. Note the significant increase in Nkchit1b transcript in mRNA from the induced traps (lane 1) and the additional chitinase product appearing in the induced traps using group 2 degenerate primers (lane 12).

FIG. 21 is a physical map of the plasmid pPCV702-chit1\chit2-HA. The plasmid is an Agrobacterium shuttle vector carrying the Nkchit1b-I or Nkchit2b-II gene, translationally fused to the HA peptide tag sequence and driven by the tandem constitutive CaMV 35S promoter. Note the presence of nptII selectable marker. Plasmid size is 12.33 kb.

FIG. 22 illustrates a Western blot demonstrating the expression and correct processing of Nkchit1b-gI-HA fusion protein in transgenic tobacco plants. Extracts of leaf tissue (0.5 g) from six transgenic plants (lanes 1-6) produced by Agrobacterium-mediated transformation of tobacco leaf discs with pPCV702-chit1-HA, as described in the Examples section, a wild type plant (negative control, lane NN) and a transgenic Serratia chitinase-HA expressing plant (positive control, lane 7) were prepared as detailed in Methods, and separated on a 12% SDS-PAGE gel. Proteins were blotted onto PVDF membrane, and fusion proteins were detected via probing with polyclonal rat anti-HA antibodies and visualization with Alkaline Phosphatase-conjugated affinity purified Goat anti-rat IgG (black bands). Note the presence of a 36 kDa band representing varying levels of expression of novel Nepenthes chitinase fusion protein (arrowhead, lanes 3, 4 and 5), and the positive identification of 59 kDa Serratia chitinase-HA fusion protein (lane 7).

FIG. 23 illustrates a Western blot demonstrating the expression and correct processing of Nkchit2b-gII-HA fusion protein in transgenic tobacco plants. Extracts of leaf tissue (0.5 g) from five transgenic plants (lanes 1-5) produced by Agrobacterium-mediated transformation of tobacco leaf discs with pPCV702-chit2-HA, as described in the Examples section, a wild type plant (negative control, lane NN) and a transgenic Serratia chitinase-HA expressing plant (positive control, lane 6) were prepared and separated as detailed in FIG. 22 above. Blotting and detection of proteins was performed as detailed in FIG. 22. Note the presence of a 32.7 kDa band representing varying levels of expression of novel Nepenthes chitinase fusion protein (arrowhead, lanes 2 and 4), and the positive identification of 59 kDa Serratia chitinase-HA fusion protein (lane 6).

FIG. 24 is a native chitinase activity gel illustrating multiple forms of novel chitinase activity in the traps of the carnivorous plants Dionea, Sarracenia and Drosera. Trap tissue extracts were prepared from the carnivorous plants Dionea, Sarracenia and Drosera by homogenization in 1.4, 1.6 or 1.9 ml extraction buffer (0.125 M Tris-HCl, pH 7.0 and 20% glycerol) per gram fresh weight, respectively. Trap tissue extracts (60—lane 1, 100 μl—lane 2 and 140 μl—lane 3) and 0.6 μg extract of E. coli overexpressing Serratia ChiA II (lane 4) were separated on native 15% PAGE, overlayed with a chitinase activity gel containing 0.01% (w/v) glycol chitin, incubated overnight at 37° C., stained 5 minutes with 0.01% (w/v) Calcofluor white M2R. Chitinase activity was detected by UV illumination (320 nm) (dark bands of lytic activity). Note the presence of multiple forms of significant chitinase activity in trap tissue extracts from all three carnivorous plants (lanes 1, 2 and 3 in all gels).

FIG. 25 is a multiple sequence alignment of the deduced partial amino acid sequences of Drosera (SEQ ID NO:7) and Dionea (SEQ ID NO:8) chitinase genes and homologous plant chitinase gene bank sequences. Two deduced Nepenthes chitinase amino acid sequences (ch1 and ch2, representing Nkchit 1b and Nkchit 2b, respectively) are included. NCBI Accession numbers of sequences of known chitinases are: Allium—Allium sativum (M94105); Potato—Solanum tuberosum (X67693); Medicago—Medicago trucatula (Y10373); and Pisum—Pisum sativum (L37876). Note extensive regions of homology.

FIGS. 26a-b illustrate protein quantification evaluation of Serratia marcescens chitinase and Nepenthes trap soup chitinase. The indicated proteins were resolved by gel electrophoresis and visualized by silver staining. FIG. 26a shows the indicated volumes of commercially available Serratia marcescens (58 kDa) and specified amounts of bovine serum albumine (BSA, 66 kDa). FIG. 26b shows the indicated volumes of Nepenthes trap soup chitinase and specified amounts of carbonic anhydrase (29 kDa). SM—indicates molecular weight marker.

FIGS. 27a-b depicts the results of chitinase activity assay performed with Serratia marcescens chitinase and Nepenthes trap soup chitinase. Chitinase activity was determined for 100 ng of commercial Serratia marcescens chitinase (FIG. 27a) and for 20-30 ng of trap soup chitinase (FIG. 27b). The tetramer p-nitrophenyl-β-D-N-N′-N″-triacetylchitotriose was used as a substrate and p-nitrophenyl release was determined.

### DESCRIPTION OF THE PREFERRED EMBODIMENTS

The present invention is of chitinases and chitinase containing compositions which are derived from carnivorous plants, polynucleotide sequences encoding such chitinases and methods of isolating and using such chitinases and chitinase compositions to reduce susceptibility of plants to chitin-containing pathogens, such as soil fungi and nematodes, to render plants refractory to chilling and frost conditions and to treat individuals suffering from diseases or conditions associated with a chitin-containing pathogen, such as Candida albicans.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings described in the Examples section hereinbelow. The invention is capable of other embodiments or of being practiced or carried out in various ways. Also, it is to be understood that the phraseology and terminology employed herein is for the purpose of description and should not be regarded as limiting.

Plants are often susceptible to diseases caused by a variety of pathogens including soil-fungi and nematodes. These diseases may cause multiple growth defects including pre- and post-emergence seedling damping-off, root-rots, crown-rots, lesions, vascular wilts and a variety of other forms of symptoms, which often result in the destruction of entire crops.

Various approaches are currently available for controlling disease associated fungi and nematodes. These methods are often based on the degradation or disruption of chitin, the major constituent of fungi cell walls and the outer covering substance of insects, nematodes, nematode eggs or nematode cysts.

Thus, a long practiced method is chemical treatment of soil or plants with fungicides or nematicides. Another method is application of certain mutant bacterial strains or naturally occurring bacterial strains, which inhibit or interfere with pathogen growth, by producing of chitin degrading enzymes also termed chitinases.

While the former approach is limited by the harmful effects of chemical pesticides on environment and human health, the latter approach is limited by several factors.

One limitation of the latter approach, is the inability to regulate the production of chitinase in the introduced bacteria in such a way that proper amounts of chitinase are produced. Another limitation stems from the limited ability of many of chitinase-producing bacteria to colonize and persist in the rhizosphere (i.e., roots) of host plants, which is a common site for plant-pathogen interaction. Root chitinase production of bacteria is also limited by the presence of other carbon sources, e.g., metabolites which are released by the root. Although some of the above limitations can be traversed by using mutant bacterial strains, such strains often revert to forms exhibiting decreased levels of chitinase production.

Though there have been numerous reports of methods for genetically manipulating plants to express chitinase so as to overcome the above limitations, almost none of the introduced genes have displayed sufficiently high chitinase activity to impart an adequate level of pathogen resistance.

As described hereinunder and in the Examples section which follows, the present invention provides novel and highly active chitinases which are derived from carnivorous plants.

Although carnivorous plants have been previously shown to secrete a base fluid (also referred to herein as “soup”) containing super active proteolytic enzymes, which are capable of breaking down the exoskeleton of trapped insects, and degrading its protein content [Owen T P and Lennon K A (1999) American J. of Bot. 86:1382-1390], it has not been previously shown that carnivorous plant tissue or base soup includes chitinases. As is further detailed hereinunder, the present inventors were able to identify and isolate chitinase polypeptides and chitinase encoding polynucleotides of several carnivorous plants.

The isolated enzymes exhibit high chitinase activity and as such can be used in diverse commercial applications as potent inhibitors of chitin-containing pathogens of both plants and mammals, as putative bio-anti-freeze substances and as possible sweeteners of fruits.

Thus, according to one aspect of the present invention there is provided an enzymatic composition, which includes a protein extract prepared from tissue or secretions of a carnivorous plant, such as, for example, Nepenthes ssp. and which exhibits endo-chitinase activity.

As used herein, the phrase “carnivorous plant” refers to plants adapted to attract and capture and digest primarily insects but also other small animals. Examples of carnivorous plants include, but are not limited to, Nepenthes ssp., Drosera sp., Dionea sp. and Sarracenia sp.

As used herein the phrase “endo-chitinase activity” refers to the ability of a hydrolytic enzyme to cleave the internal beta-1,4 glycosidic linkages in chitin molecules to liberate oligomers of at least 3 GluNAc units.

As used herein the phrase “protein extract” refers to a preparation, which includes proteins. This may include solid plant extracts, liquid plant extracts, hydrophilic plant extracts, lipophilic plant extracts, individual plant constituents and mixtures thereof. The protein extract of the present invention may be a purified protein extract, a partially purified protein extract or a crude protein extract, as long as such an extract exhibits endo-chitinase activity.

According to one preferred embodiment of the present invention, the enzymatic composition is preferably derived from trap or leaf tissue or trap secretions (e.g., trap soup) of Nepenthes ssp. and includes at least one protein which exhibits an endo-chitinase activity (also referred to herein as the “active fraction” of the protein extract).

The example section which follows provides a comprehensive analysis of the biochemical, immunological and functional properties which characterize the proteins of the enzymatic composition of the present invention.

The following section describes biochemical and immunological properties of the endo-chitinase active proteins of the enzymatic composition of the present invention:

Molecular weight—the endo-chitinase proteins of the present invention may be active as monomers with an apparent molecular weight of about 24 to 27 kDa, about 30 to 31 kDa, about 31 to 33 kDa, about 32 to 36 kDa, about 34 to 38 kDa, as determined via gel electrophoresis under reducing and denaturing conditions. The polypeptides of the present invention may also be assembled as multisubunit proteins such as dimmers or trimers consisting of homologous or heterologous subunits. As such the proteins of the present invention are characterized with an apparent molecular weight of about 48 to 54 kDa, about 60 to 62 kDa, about 62 to 66 kDa, about 64 to 72 kDa, about 68 to 76 kDa, about 76 kDa to about 100 kDa, as determined via gel electrophoresis under non-reducing conditions.

Endo-chitinase activity—the proteins of the present invention are characterized with an endochitinase activity as determined using assay-specific substrates (see Example 3 of the Examples section) and monitoring nitrophenol release by spectrophotometric analysis at 410 mm.

Km—the proteins of the present invention may be considered as having high Km values as compared to Serratia marcescens chitinase, however it is presumed that these polypeptides apparently don't follow the Michaelis-Menten model, as a sigmoidal plot of reaction velocity versus substrate concentration is observed, suggesting that the polypeptides of the present invention are allosteric enzymes. This is substantiated by the observations that chitin induction significantly increases the enzymatic activity of the polypeptides and also by the observation that the enzymes of the present invention may include more than one subunit (see Example 20 of the Examples section).

pI—The pI value of the endo-chitinase proteins of the present invention is below 10, preferably between 7-9, as determined by the binding to an FPLC anion exchange column at the presence of carbonate buffer having pH 10. Moreover the observation that chitinase activity could be detected already in fractions eluted at 200 mM NaCl, suggests that the pI value is relatively high (see Example 6 of the Examples section).

Antibody reactivity—Unlike trap-tissue and leaf derived chitinase proteins, the trap-soup chitinase proteins of the present invention are not reactive with a polyclonal antibody directed to Serratia marcescens chitinase (ChiAII), indicating that the proteins do not share antigenic epitopes with the Serratia marcescens chitinase (see Example 2 of the Examples section).

Enzymatic stability—generally, the endo-chitinase proteins of the enzymatic composition of the present invention retains enzymatic activity following incubation at 50° C. for 30 minutes at pH 6.7 or is active after incubation at 37° C. for 16 hours at pH 5.

The enzymatic compositions of the present invention further exhibit a strong anti-fungal activity (see Examples 9-11 of the Examples section).

As used herein the phrase “anti-fungal activity” refers to a fungistatic activity, which prevents further fungal growth and/or a fungicidal activity, which promotes killing of fungi already present. As is evident from the results presented in the Examples section which follows, the enzymatic compositions of the present invention exhibit efficient and enhanced fungocidic activity as compared to prior art chitinase enzymes (see Examples 9-11 of the Examples section).

In contrast to Serratia chitinase, the anti-fungal activity of the enzymatic composition of the present invention is fungicidal and as such can be used for both agricultural and therapeutic purposes.

Chitinases are known to play a major role in plant defense response by hydrolyzing chitin-containing fungal cell walls. This hydrolytic activity slows down fungal growth and delays or avoids the invasion of pathogens into plant tissues. Since the enzymatic compositions of the present invention exhibit extremely high anti-fungal activity (see Examples 9-11 of the Examples section) such compositions can be used for treating infections caused by chitin containing pathogens in both humans and animals (e.g., canines, felines, ovines, porcines, equines, bovines, humans and the like) and for disinfecting plants and plant-derived tissues.

The enzymatic compositions of the present invention are particularly advantageous as possible therapeutic tools, given the poor potency of currently available anti-fungal drugs. For example, the only effective treatment of Candida albicans infections is intravenous administration of amphotericin B, which often results in serious adverse affects that are accompanied by hypotension and collapse.

When used for treating fungal/bacterial infections in humans or animals, the enzymatic composition of the present invention is preferably included, as the active ingredient in a pharmaceutical composition preferably designed for topical or oral administration.

The term “treating” refers to alleviating or diminishing a symptom associated with a bacterial infection. Preferably, treating cures, e.g., substantially eliminates, the symptoms associated with the infection and/or substantially decreases bacterial load in the infected tissue.

As used herein a “pharmaceutical composition” refers to a composition of one or more of the active ingredients described hereinabove, or physiologically acceptable salts or prodrugs thereof, with other chemical components such as physiologically suitable carriers and excipients. The purpose of a pharmaceutical composition is to facilitate administration of a compound to an organism.

Hereinafter, the phrases “pharmaceutically acceptable carrier” and “physiologically acceptable carrier” are used interchangeably to refer to a carrier or a diluent that does not cause significant irritation to a treated individual and does not abrogate the biological activity and properties of the active ingredient.

Herein the term “excipient” refers to an inert substance added to a pharmaceutical composition to further facilitate administration of active ingredients. Examples, without limitation, of excipients include calcium carbonate, calcium phosphate, various sugars and types of starch, cellulose derivatives, gelatin, vegetable oils and polyethylene glycols.

Techniques for formulation and administration of the pharmaceutical compositions of the present invention may be found in “Remington's Pharmaceutical Sciences,” Mack Publishing Co., Easton, Pa., latest edition, which is incorporated herein by reference.

Suitable routes of administration may, for example, include oral, rectal, transmucosal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a pharmaceutical composition in a local rather than systemic manner, for example, via injection of the composition directly into the area of infection often in a depot or slow release formulation, such as described below.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredient into compositions which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the pharmaceutical composition can be formulated by combining the active agents with pharmaceutically acceptable carriers well known in the art. Such carriers enable the pharmaceutical composition used by the method of the invention to be formulated as tablets, pills, dragees, capsules, liquids, gels, syrups, slurries, suspensions, and the like, for oral ingestion by a patient. Pharmacological compositions for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose compositions such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the agents for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the active ingredient and a suitable powder base such as lactose or starch.

Ophthalmic formulations, eye ointments, powders, solutions and the like, are also contemplated as being within the scope of this invention.

The compositions described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active ingredient in water-soluble form. Additionally, suspensions of the active ingredient may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or formulations, which increase the solubility of the active ingredient to allow for the composition of highly concentrated solutions.

Alternatively, the active ingredient may be in powder form for constitution with a suitable vehicle, e.g., sterile, pyrogen-free water, before use.

The composition of the present invention may also be formulated in rectal compositions such as suppositories or retention enemas, using, e.g., conventional suppository bases such as cocoa butter or other glycerides.

In addition to the formulations described previously, a composition of the present invention may also be formulated for local administration, such as a depot composition. Such long acting formulations may be administered by implantation (for example subcutaneously or intramuscularly) or by intramuscular injection. Thus, for example, the composition may be formulated with suitable polymeric or hydrophobic materials (for example, as an emulsion in an acceptable oil) or ion exchange resins, or as sparingly soluble derivatives such as sparingly soluble salts. Formulations for topical administration may include, but are not limited to, lotions, suspensions, ointments gels, creams, drops, liquids, sprays emulsions and powders.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of active ingredient effective to prevent, alleviate or ameliorate symptoms of disease or prolong the survival of the subject being treated.

Determination of a therapeutically effective amount is well within the capability of those skilled in the art, especially in light of the detailed examples provided herein (see Example 9-11 and 20 of the Examples section).

The therapeutically effective amount or dose can be estimated initially from cell culture assays and cell-free assays (See Example 9-11 and 20 of the Examples section).

Since the enzymatic compositions of the present invention exhibit high anti-fungal activity (see Examples 9-11 of the Examples section below) low concentrations thereof can be used in treatment of various fungal diseases, thereby avoiding cytotoxicity.

Regardless, toxicity and therapeutic efficacy of the pharmaceutical compositions described herein can be determined by standard pharmaceutical procedures in experimental animals, e.g., by determining the IC₅₀ and the LD₅₀ (lethal dose causing death in 50% of the tested animals) for a subject ingredient. The data obtained from assays can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active ingredient, which are sufficient to maintain the required effects, termed the minimal effective concentration (MEC). The MEC will vary for each composition, but can be estimated from in vitro data; e.g., the concentration necessary to achieve 50-90% inhibition (see Example 1 of the Examples section). Dosages necessary to achieve the MEC will depend on individual characteristics and route of administration. HPLC assays or bioassays can be used to determine plasma concentrations.

Dosage intervals can also be determined using the MEC value. Compositions should be administered using a regimen, which maintains plasma levels above the MEC for 10-90% of the time, preferable between 30-90% and most preferably 50-90%.

It is noted that, in the case of local administration or selective uptake, the effective local concentration of the drug may not be related to plasma concentration. In such cases, other procedures known in the art can be employed to determine the effective local concentration.

Depending on the severity and responsiveness of the infection to be treated, dosing can also be a single administration of a slow release composition, with course of treatment lasting from several days to several weeks or until cure is effected or diminution of the infection state is achieved.

The amount of a composition to be administered will, of course, be dependent on the subject being treated, the severity of the infection, the manner of administration, the judgment of the prescribing physician, etc.

Compositions of the present invention can be packaged in a dispenser device, as one or more unit dosage forms as part of an FDA approved kit, which preferably includes instruction for use, dosages and counter indications. The kit can include, for example, metal or plastic foil, such as a blister pack suitable for containing pills or tablets, or a dispenser device suitable for use as an inhaler. The kit may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising an active ingredient suitable for use with the present invention may also be prepared, placed in an appropriate container, and labeled for treatment of an indicated disease or condition.

The above described pharmaceutical compositions can be used to treat a variety of chitin-containing pathogen infections including, but not limited to fungal infections (e.g., Cutaneous mycoces Subcutaneous mycoces Pulmonary mycoces Candidiasis), protozoal infections [e.g., Toxoplasmosis Malaria (Plasmodium species) Leishmaniasis (Leishmania species) Chagas disease, sleeping sickness (Trypanosoma species)] and helminth (worm) infections (e.g., Schistosomiasis Trichinosis Filariasis Ochocerciasis Fungal Infections).

The enzymatic compositions of the present invention can find also significant use as pesticides, repelling or killing chitin-containing pathogens including fungi, nematodes, insects and disease agents. For example, fungal pathogens include fungal species from a wide variety of genera, including Fusarium, Pythium, Phytophthora, Verticillium, Rhizoctonia, Macrophomina, Thielaviopsis, Sclerotinia and the like. Plant diseases caused by fungi include pre and post-emergence seedling damping-off, hypocotyl rots, root rots, crown rots, vascular wilts and a variety of other forms of symptom development. Nematode pathogens include but are not limited to nematode species from the genera Meloidogyne, Heterodera, Ditylenchus, Pratylenchus. Plant diseases caused by nematodes include but are not limited to root galls, root rot, lesions, “stubby” root, stunting, and various other rots and wilts associated with increased infection by pathogenic fungi. Some nematodes (e.g., Trichodorus, Lonaidorus, Xiphenema) can serve as vectors for virus diseases in a number of plants including Prunus, grape, tobacco and tomato. It will be appreciated that these compostions can also be used as biological anti-freeze substances, protecting plants from cold damage, and as possible sweeteners of fruits, as fill be described in details hereinunder.

Thus the enzymatic compositions of the present invention can also be included in agricultural compositions, which also preferably include an agricultural acceptable carrier.

An agriculturally acceptable carrier can be a solid or a liquid, preferably a liquid, more preferably water. While not required, the agricultural composition of the invention may also contain other additives such as fertilizers, inert formulation aids, i.e. surfactants, emulsifiers, defoamers, dyes, extenders and the like. Reviews describing methods of preparation and application of agricultural compositions are available. See, for example, Couch and Ignoffo (1981) in Microbial Control of Pests and Plant Disease 1970-1980, Burges (ed.), chapter 34, pp. 621-634; Corke and Rishbeth, ibid, chapter 39, pp. 717-732; Brockwell (1980) in Methods for Evaluating Nitrogen Fixation, Bergersen (ed.) pp. 417-488; Burton (1982) in Biological Nitrogen Fixation Technology for Tropical Agriculture, Graham and Harris (eds.) pp. 105-114; and Roughley (1982) ibid, pp. 115-127; The Biology of Baculoviruses, Vol. II, supra, and references cited in the above. Wettable powder compositions incorporating baculoviruses for use in insect control are described in EP 697,170 incorporated by reference herein.

Preferred methods of applying the agricultural compositions of the present invention are leaf application, seed coating and soil application, as disclosed in U.S. Pat. No. 5,039,523, which is fully incorporated herein.

The importance and commercial applicability of the chitinase-containing carnivorous-plant compositions of the present invention has led the present inventors to identify and isolate the polynucleotides encoding such endochitinases from carnivorous plants.

Thus, according to another aspect of the present invention there is provided a genomic complementary or composite polynucleotide sequence which is isolated from carnivorous plant tissue, and which encodes a polypeptide exhibiting endo-chitinase activity either in itself (monomer) or as part of a multimeric protein.

As used herein the phrase “complementary polynucleotide sequence” refers to sequences, which originally result from reverse transcription of messenger RNA using a reverse transcriptase or any other RNA dependent DNA polymerase. Such sequences can be subsequently amplified in vivo or in vitro using a DNA dependent DNA polymerase.

As used herein the phrase “genomic polynucleotide sequence” refers to sequences, which are derived from a chromosome and thus reflect a contiguous portion of a chromosome.

As used herein the phrase “composite polynucleotide sequence” refers to sequences, which are at least partially complementary and at least partially genomic. A composite sequence can include some exonal sequences required to encode the polypeptide of the present invention, as well as some intronic sequences interposed in between the exonal sequences. The intronic sequences can be of any source and typically include conserved splicing signal sequences. Such intronic sequences may further include cis acting expression regulatory elements.

According to one preferred embodiment of this aspect of the present invention the isolated polynucleotide of the present invention encodes a polypeptide, which is at least 70%, at least 75%, at least 80%, at least 85%, at least 90%, at least 95% or more, say 95%-100% identical to SEQ ID NO: 5.

Such identity and/or sequence homology may be determined using computer dedicated softwares such as the BestFit software of the Wisconsin sequence analysis package which utilizes the Smith and Waterman algorithm and the following parameters: gap creation penalty equals 8 and gap extension penalty equals 2.

According to another preferred embodiment of this aspect of the present invention the isolated polynucleotide of the present invention encodes a polypeptide which is at least 75%, at least 80%, at least 85%, at least 90%, at least 95% or more, say 95%-100% identical to SEQ ID NO: 6.

According to another preferred embodiment of this aspect of the present invention the isolated polynucleotide of the present invention encodes a polypeptide which is at least 81%, at least 85%, at least 90%, at least 95% or more, say 95%-100% identical to SEQ ID NO: 7.

According to another preferred embodiment of this aspect of the present invention the isolated polynucleotide of the present invention encodes a polypeptide which is at least 77%, at least 80%, at least 85%, at least 90%, at least 95% or more, say 95%-100% identical to SEQ ID NO: 8.

According to yet another preferred embodiment of this aspect of the present invention the encoded polypeptide has a signal peptide of at least 30 amino acids, at least 32 amino acids, at least 34 amino acids at least 36, say 38 amino acids. Such a signal peptide is set forth in SEQ ID NO: 47 and is presumingly used for protein secretion (see Example 14 of the Examples section).

According to still another preferred embodiment of this aspect of the present invention the encoded polypeptide has a proline rich region characterized by at least 10 and no more than 15 proline amino acids (see SEQ ID NO: 49). These prolines serve as putative glycosylation sites and may be important for protein secretion and protein interactions [Liu et al. J. Biomed Sci March 1994;1(2):65-82].

According to another preferred embodiment the polynucleotide according to this aspect of the present invention is as set forth in SEQ ID NOs: 1, 2, 3 or 4 or an active portion thereof. As used herein the phrase “active portion” refers to a portion of the chitinase, which retains chitinase activity (i.e., catalytic domain) and/or substrate recognition (i.e., cysteine rich domain).

Alternatively or additionally, the polynucleotide according to this aspect of the present invention is hybridizable with SEQ ID NOs: 1, 2, 3 or 4.

Hybridization for long nucleic acids (e.g., above 200 bp in length) is effected preferably under stringent or moderate hybridization, wherein stringent hybridization is effected by a hybridization solution containing 10% dextrane sulfate, 1 M NaCl, 1% SDS and 5×10⁶ cpm ³²p labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×106 cpm ³²p labeled probe, at 65° C., with a final wash solution of 1×SSC and 0.1% SDS and final wash at 50° C.

Thus, this aspect of the present invention provides polynucleotides, which encode polypeptides exhibiting endo-chitinase activity. The isolated polynucleotides of the present invention can be expressed in variety of single cell or multicell expression systems and the recombinant polypeptides recovered therefrom used in pharmaceutical and agricultural applications as described hereinabove with respect to the enzymatic composition of the present invention.

For expression in a single cell system, the polynucleotides of the present invention are cloned into an appropriate expression vector (i.e., construct).

Depending on the host/vector system utilized, any of a number of suitable transcription and translation elements including constitutive and inducible promoters, transcription enhancer elements, transcription terminators, and the like., can be used in the expression vector [see, e.g., Bitter et al., (1987) Methods in Enzymol. 153:516-544].

Other then containing the necessary elements for the transcription and translation of the inserted coding sequence, the expression construct of this aspect of the present invention can also include sequences engineered to enhance stability, production, purification, yield or toxicity of the expressed polypeptide. For example, the expression of a fusion protein or a cleavable fusion protein comprising a polypeptide of the present invention and a heterologous protein can be engineered. Such a fusion protein can be designed so as to be readily isolated by affinity chromatography; e.g., by immobilization on a column specific for the heterologous protein. Where a cleavage site is engineered between the protein of interest (i.e., chitinase) and the heterologous protein, chitinase protein can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

A variety of cells can be used as host-expression systems to express the chitinase coding sequence. These include, but are not limited to, microorganisms, such as bacteria transformed with a recombinant bacteriophage DNA, plasmid DNA or cosmid DNA expression vector containing the chitinase coding sequence; yeast transformed with recombinant yeast expression vectors containing the chitinase coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the alkaline chitinase coding sequence (further described in the specifications hereinunder). Mammalian expression systems can also be used to express the chitinases. Bacterial systems are preferably used to produce recombinant chitinase, according to the present invention, thereby enabling a high production volume at low cost.

In bacterial systems, a number of expression vectors can be advantageously selected depending upon the use intended for chitinase expressed. For example, when large quantities of chitinase are desired, vectors that direct the expression of high levels of protein product, possibly as a fusion with a hydrophobic signal sequence, which directs the expressed product into the periplasm of the bacteria or the culture medium where the protein product is readily purified may be desired. Certain fusion protein engineered with a specific cleavage site to aid in recovery of the chitinase may also be desirable. Such vectors adaptable to such manipulation include, but are not limited to, the pET series of E. coli expression vectors [Studier et al. (1990) Methods in Enzymol. 185:60-89).

It will be appreciated that when codon usage for chitinase cloned from plants is inappropriate for expression in E. coli, the host cells can be co-transformed with vectors that encode species of tRNA that are rare in E. coli but are frequently used by plants. For example, co-transfection of the gene dnaY, encoding tRNA_{ArgAGA/AGG}, a rare species of tRNA in E. coli, can lead to is high-level expression of heterologous genes in E. coli. [Brinkmann et al., Gene 85:109 (1989) and Kane, Curr. Opin. Biotechnol. 6:494 (1995)]. The dnaY gene can also be incorporated in the expression construct such as for example in the case of the pUBS vector (U.S. Pat. No. 6,270,0988).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

Other expression systems such as insects and mammalian host cell systems, which are well known in the art can also be used by the present invention.

Transformed cells are cultured under conditions, which allow for the expression of high amounts of recombinant chitinase. Such conditions include, but are not limited to, media, bioreactor, temperature, pH and oxygen conditions that permit protein production. Media refers to any medium in which a cell is cultured to produce the recombinant chitinase protein of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

Recovery of the recombinant protein is effected following an appropriate time in culture. The phrase “recovering the recombinant protein refers to collecting the whole fermentation medium containing the protein and need not imply additional steps of separation or purification. Not withstanding from the above, proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

The recombinant endo-chitinase proteins of the present invention are preferably retrieved in “substantially pure” form to be used in the pharmaceutical compositions and agricultural compositions, described above. As used herein, “substantially pure” refers to a purity that allows for the effective use of the protein in the diverse applications, described hereinabove.

As is shown in Example 19 of the Examples section, which follows, numerous carnivorous plant species include expressible polynucleotides which encode endo-chitinases similar to the endo-chitinases of the present invention.

Thus, the polynucleotide and polypeptide sequences information provided by the present invention can also be used to identify and isolate additional polynucleotide sequences of carnivorous plants, which encode endo-chitinases. Such identification and isolation can be effected using molecular and biochemical methods which are well known in the art, including PCR amplification, library screening and the like (see the Examples section for further detail)

In addition, sequence information along with biochemical characteristics inherent to the polypeptides of the present invention can be used to isolate crude or purified chitinase-active fractions from other carnivorous plants.

Thus, according to an additional aspect of the present invention there is provided a method of isolating polypeptides exhibiting a high endo-chitinase activity from carnivorous plants.

The method is then effected by preparing a protein extract from a tissue (i.e., trap or leaf) or trap secretions (e.g., trap soup of pitcher plants) of the carnivorous plants.

Following protein extraction a chitinase active fraction is isolated and individual polypeptides, originating from the active fraction and exhibiting high chitinase activity are identified using chitinase activity assays, further detailed hereinunder.

Finally, polypeptides with enhanced chitinase activity may be biochemically characterized (e.g., pH and temperature sensitivity, molecular weight, activity under reducing/non-reducing conditions, pI, endo/exo-chitinase activity and the like, see the Examples section) and functionally assayed for biocidic (e.g., anti-fungal) activity.

It will be appreciated that the hereinabove described method is preferably effected by first inducing chitinase activity within the context of the whole plant as to facilitate polypeptides isolation. This may be effected by various internal and external factors such as plant hormones (e.g., ethylene), heat shock, chemicals, pathogens, oxygen lack, light, stress, and the like. A preferred induction protocol according to the present methodology is chitin induction (see Example 7 of the Examples section).

Preparation of a plant protein extract may be effected by any standard protein extraction method known in the art. Selection of a protein extraction method may depend on the tissue distribution of the active polypeptide and its cellular localization. In the case of proteins not secreted into the plant cell apoplasm or intercellular space, a mechanism for lysing the plant cell wall must be utilized in order to release and capture the protein of interest. A review on plant protein extraction methods is provided by Cunningham C and Porter A J R (1998) “Recombinant protein from plants” Humana Press Totowa N.J.

Secreted or apoplastic proteins may be extracted by simply collecting the secreted fluid, however measures must be taken not to rupture the neighboring cells to thereby expose secreted proteins to a proteolytic or denaturing environment.

Preferably, extracts of secreted or apoplastic proteins are prepared by vacuum infiltration of the tissue of interest, such as leaf or trap with 5 mm EDTA, 10 mm ascorbic acid, 10 mm mercaptoethanol, 1 mm phenylmethyl sulfonylfluoride, 2 mm caproic acid and 2 mm benzamidine. The vacuum infiltration is in accordance with the process described in Mauch and Staehelin [The plant Cell 1:447-457 (1989)]. The treated tissue is packed vertically in a funnel and placed in a centrifuge tube so as to avoid bending of the tissue. With the tissue packed in the funnel the material is centrifuged to remove without rupturing the cells of the tissue the extracellular infiltrate, which is captured in the centrifuge tube as an extract.

The recovered extract is then assayed for chitinase activity. In general, chitinase activity can be measured as the enzymatic release of glucosamine from colloidal chitin (exochitinase) and from chitin oligomers (endochitinase).

Chitinase activity may be assayed by an in-gel activity assay (see Example 1 of the Examples section). Samples (i.e., protein extract fractions) are subjected to electrophoresis in a native polyacrylamide minigel, as previously described by Blackshear (1984). Following electrophoresis the gel is overlaid with a polyacrylamide gel containing glycol chitin as a substrate and incubated under effective conditions, according to the procedure of Trudel and Asselin [(1989) Analytical Biochemistry 178:362-366]. Chitinase activity bands are detected by the absence of staining with calcofluor when viewed under ultraviolet light.

Alternatively, chitinase activity may be determined using the analog p-nitrophenyl-β-D-N,N′,N″-triacetylchitobiose (Sigma IL). The assay is effected in ELISA plates containing KH₂PO₄ buffer including CaCl₂ at pH 6.7, with the substrate dissolved in nanopure water and the protein extract fraction. Reaction is terminated following 30 minutes at 50° C. and absorbance is measured at 405 nm using an ELISA plate reader. Blanks are used to discount any absorption due to the enzyme or substrate alone. The p-nitrophenol released by the samples is calculated using a standard curve. Units of enzymatic activity are determined as the number of moles of p-nitrophenol released per minute under the assay conditions.

Once a chitinase active fraction has been identified, the polypeptides of interest may be concentrated and purified according to any suitable purification procedures (see Example 6 of the Examples section). Such procedures may include but are not limited to protein precipitation, expanded bed chromatography, ultrafiltration, anion exchange chromatography, cation exchange chromatography, hydrophobic-interaction chromatography, HPLC, FPLC and affinity chromatography (such as on chitin columns) as disclosed in U.S. Pat. No. 6,284,875, which is fully incorporated herein. A general discussion of some protein purification techniques is provided by Jervis et al., Journal of Biotechnology 11:161-198 (1989), the teachings of which are herein incorporated by reference.

Using the methodology described above, the present inventors have uncovered additional members of the endo-chitinase family of enzymes from three additional genera (Dionea sp., Drosera sp. and Sarracenia sp.) of carnivorous plants

Other than serving as a template for protein productions the chitinase encoding polynucleotides of the present invention can be also used in a variety of applications.

Thus, according to still another aspect of the present invention there is provided a method of reducing susceptibility of a plant to chitin-containing pathogens.

The method is effected by ectopically expressing within plants the highly active chitinase polypeptides of the present invention.

Polypeptide expression in plants, is effected by transforming plants with the polynucleotide sequences of the present invention.

For effecting plant transformation, the polynucleotides which encode endo-chitinases are preferably included within a nucleic acid construct or constructs which serve to facilitates the introduction of the exogenous polynucleotides into plant cells or tissues and to express these enzymes in the plant.

The nucleic acid constructs according to the present invention are utilized to express in either a transient or preferably a stable manner the chitinase encoding polynucleotide of the present invention within a whole plant, defined plant tissues, or defined plant cells.

Thus, according to a preferred embodiment of the present invention, the nucleic acid constructs further include a promoter for regulating the expression of the chitinase encoding polynucleotide of the present invention.

Numerous plant functional expression promoters and enhancers which can be either tissue specific, developmentally specific, constitutive or inducible can be utilized by the constructs of the present invention, some examples are provided hereinunder.

As used herein in the specification and in the claims section that follows the phrase “plant promoter” or “promoter” includes a promoter which can direct gene expression in plant cells (including DNA containing organelles). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric, i.e., formed of portions of at least two different promoters.

Thus, the plant promoter employed can be a constitutive promoter, a tissue specific promoter, an inducible promoter or a chimeric promoter.

Examples of constitutive plant promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

Examples of tissue specific promoters include, without being limited to, bean phaseolin storage protein promoter, DLEC promoter, PHSβ promoter, zein storage protein promoter, conglutin gamma promoter from soybean, AT2S1 gene promoter, ACT11 actin promoter from Arabidopsis, napA promoter from Brassica napus and potato patatin gene promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

The construct according to the present invention preferably further includes an appropriate and unique selectable marker, such as, for example, an antibiotic resistance gene. In a more preferred embodiment according to the present invention the constructs further include an origin of replication.

The constructs according to the present invention can be a shuttle vector, which can propagate both in E. coli (wherein the construct comprises an appropriate selectable marker and origin of replication) and be compatible for propagation in cells, or integration in the genome, of a plant.

There are various methods of introducing nucleic acid constructs into both monocotyledonous and dicotyledenous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct in which case these sequences are not inherited by a progeny of the plant.

There are two principle methods of effecting stable genomic integration of exogenous sequences such as those included within the nucleic acid constructs of the present invention into plant genomes:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Following transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Transient expression methods which can be utilized for transiently expressing the isolated nucleic acid included within the nucleic acid construct of the present invention include, but are not limited to, microinjection and bombardment as described above but under conditions which favor transient expression, and viral mediated expression wherein a packaged or unpackaged recombinant virus vector including the nucleic acid construct is utilized to infect plant tissues or cells such that a propagating recombinant virus established therein expresses the non-viral nucleic acid sequence.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al, Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, the constructions can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that these sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

It will be appreciated that co-transformation of the polynucleotides of the present invention together with other polynucleotides is desirable to achieve a synergistic effect, such as the combination of chitinases and gluconases, as disclosed in EP NO: 440,304 A1, which is fully incorporated herein.

Any plant species may be transformed with the nucleic acid constructs of the present invention including species of gymnosperms as well as angiosperms, dicotyledonous plants as well as monocotyledonous plants which are commonly used in agriculture, horticulture, forestry, gardening, indoor gardening, or any other form of activity involving plants, either for direct use as food or feed, or for further processing in any kind of industry, for extraction of substances, for decorative purposes, propagation, cross-breeding or any other use.

Generally, after transformation plant cells or explants are selected for the presence of one or more markers, which are encoded by the constructed vector of the present invention, whereafter the transformed material is regenerated/propagated into a whole plant. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transgenic plant be produced such that the regenerated plant has the identical traits and characteristics of the parent transgenic plant, e.g., a reproduction of the fusion protein. Therefore, it is preferred that the transgenic plant be regenerated by micropropagation which provides a rapid, consistent reproduction of the transgenic plants.

Micropropagation is a process of growing new generation plants from a single piece of tissue that has been excised from a selected parent plant or cultivar. This process permits the mass reproduction of plants having the preferred tissue expressing the fusion protein. The new generation plants, which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transgenic plantlets are transferred to a greenhouse for hardening where the plants' tolerance to light is gradually increased so that it can be grown in the natural environment.

Following plant transformation and propagation, selection of appropriate plants can be effected by monitoring the expression levels of the exogenous endo-chitinase or by monitoring the transcription levels of the corresponding mRNA.

The expression levels of the exogenous endo-chitinase can be determined using immunodetection assays (i.e., ELISA and western blot analysis, immunohistochemistry and the like), which may be effected using antibodies specifically recognizing the recombinant polypeptide, such as an antibody directed to the N-terminus end of the signal peptide of SEQ ID NO: 47. Methods of antibody generation are disclosed in “Cellular and Molecular immunology” Abbas, K. et al. (1994) 2nd ed. W B Saunders Comp ed. which is fully incorporated herein. Alternatively, the recombinant polypeptides can be monitored by SDS-PAGE analysis using different staining techniques, such as but not limited to, coomassie blue or silver staining.

mRNA levels of the polypeptides of the present invention may also be indicative of the transformation rate and/or level. mRNA levels can be determined by a variety of methods known to those of skill in the art, such as by hybridization to a specific oligonucleotide probe (e.g., Northern analysis) or PCR.

Such polypeptides are of at least 17, at least 18, at least 19, at least 20, at least 22, at least 25, at least 30 or at least 40, bases specifically hybridizable with the polynucleotide sequences described hereinabove.

To specifically detect the polynucleotide sequences of the present invention, measures are taken to design specific oligonucleotide probes, which would not hybridize with other related genes under the hybridization conditions used. Example 14 illustrates conserved sequences, which may be useful for the design of specific oligonucleotides.

Hybridization of short nucleic acids (below 200 bp in length, e.g. 17-40 bp in length) can be effected by the following hybridization protocols depending on the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 37° C., final wash solution of 6×SSC and final wash at 22° C.

The oligonucleotides of the present invention can be used in any technique which is based on nucleotide hybridization including, subtractive hybridization, differential plaque hybridization, affinity chromatography, electrospray mass spectrometry, northern analysis, RT-PCR and the like. For PCR-based methods a pair of oligonucleotides is used in an opposite orientation so as to direct exponential amplification of a portion thereof in a nucleic acid amplification reaction, such as a polymerase chain reaction. The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Plants and plant parts producing or over-producing the novel chitinases according to this aspect of the present invention, either alone or in combination with other genes encoding proteins which work synergistically with the recombinant chitinases of the present invention (described hereinabove), may be used to evaluate pathogen resistance, in particular fungal resistance. Subsequently, the more resistant lines may be used in breeding programs to yield commercial varieties with enhanced pathogen, in particular fungal, resistance. Plants with reduced susceptibility against pathogen or fungal attack, may be used in the field or in greenhouses, and subsequently can be used for animal feed, direct consumption by humans, for prolonged storage, used in food- or other industrial processing, and the like. The advantages of the plants, or parts thereof, produced according to the present invention are a reduced need for fungicide treatment, lowering costs of material, labour, and environmental pollution, or prolonged shelf-life of products (e.g. fruit, seed, and the like). Furthermore, post-harvest losses may be reduced due to the presence of the chitinases expressed by harvested plants or plant tissues.

The present methodology may also be used in protecting plants from cold damage. Chitinases are also known to degrade chitin into soluble sugar units (e.g., N-acetyl-glucosamine monomers or small oligomers of same) [Roberts et al. (1988) J. Gen. Microbiol. 134, 169-176]. Small soluble compounds, in particular sugars, are known to be associated with or causative of protection against chilling or freezing damage [Finkle, B. J. et al. (1985) Cryopreservation of Plant Cells and Organs (Chapter 5), Pages 75-113, CRC Press, Inc. Boca Raton, Fla.; Sakai, et al. (1968) Cryobiol. 5(3):160-174]. It is thus believed that cold damage protection can be mediated by the chitinases of the present invention which may degrade plant polysaccharides (e.g., cleavage of beta-1,4 glycosidic bonds in the polysaccharide components of the cell wall such as hemicellulose and pectin) to yield increased levels of soluble sugars (monomers or small oligomers) which in turn results in enhanced protection against freezing or chilling damage.

Thus according to yet another aspect of the present invention, there is provided a method of reducing susceptibility of plants to cold damage.

The method comprises transforming plants with the polynucleotides of the present invention, as described hereinabove and, growing the transformed plants in field conditions under which they are subjected to chilling temperatures (0-10° C.) or freezing temperatures (0° C. or below), and selecting the plants (or their fruit) which display reduced chilling or freezing damage or which otherwise display resistance or increased resistance to chilling or freezing damage (see U.S. Pat. Nos. 6,235,683, 5,776,448, 5,633,450 and 5,554,524, each of which is herein incorporated by reference in its entirety).

This method may be used to generate plants, which are protected against cold damage (i.e., freezing or chilling).

Given the enhanced levels of soluble sugars contained in the transformed plants of the present invention, the method of the invention may also be used to create plants which yield fruits having a higher sugar content. In such cases, the exogenous chitinase-encoding polynucleotides are preferably expressed in plant parts in which an increased sugar content is desired (e.g., fruit).

The method of the invention may be further used to confer other properties on plants associated with reducing sugars content or elevated reducing sugars content, including improved storage, preservation and shelf-life properties.

The present invention may also have additional related applications. Chitinases of the present invention can be used as a tool to degrade injected or implanted chitin-based structures for medical purposes. For example, drugs could be incorporated in chitin based capsules (‘chitosomes’). The concomitant presence of well defined amounts of the chitinases of the present invention in the capsule could ensure a controlled release of drugs. A slow but gradual release of drug could particularly be envisioned when it is trapped in a chitin matrix. The use of the chitinase enzyme in such a system would result in ultimate destruction of the chitin-based capsule and not elicit an immunological response. The drugs used in such a system could vary from small compounds to proteins and DNA fragments for the purpose of enzyme and gene therapy.

Another, related, application is the use of the chitinases of the present invention, preferably the recombinant form, for the swift degradation of implants that contain chitin as a structural component. This would be useful in the case of implants that only temporarily have to fulfill a function and can be conveniently ‘dissolved’ by administration of recombinant chitinase.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immununology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND METHODS

Plant material: Pitcher plants (Nepenthes kassiana) were grown in the greenhouse (25° C.) without fertilizer and watered with double distilled water. Trap soup was removed from closed traps by a sterile syringe in order to preserve sterility and stored at −70° C. in aliquots. When trap and leaf tissue were used, they were prepared as follows: leaf or trap tissue were homogenized in 2 or 5 ml extraction buffer (0.125 M Tris-HCl, pH 7.0 and 20% glycerol) per 1 g fresh weight, respectively. The homogenate was centrifuged 5 minutes at 14,000 rpm in an Eppendorf minifuge and the supernatant (leaf or trap tissue extract) was used for the SDS-PAGE and chitinase activity gel analyses.

Chitinase activity gels: Chitinase activity was studied by separating extracts prepared from leaves, trap tissue or sterile trap soup on a native 12% acrylamide gel overlayed after the run with an additional acrylamide gel containing chitin-glycol (0.01% w/v) as a substrate and incubated for 8 h at 37° C. Chitinase activity was visualized under UV light (260 nm) as dark spots on the gel after staining for 5 minutes with 0.01% (w/v) Calcofluor white M2R.

Western analysis. SDS-PAGE was performed with 12 or 15% resolving gel and 5% stacking gel. The protein was transferred onto PVDF membrane (Gelman) and the chitinase was detected using either rabbit polyclonal antibodies against Serratia marcescens chitinase (ChiAII) (Jones et al., 1986) or rat anti-HA antibodies (in the case of transgenic expression in plants) [Rat monoclonal antibody (clone 3F10), Roch, Cat. No. 1867423] and then visualized by Alkaline Phosphatase-conjugated affinity purified goat anti-rabbit or anti-rat IgG (Affinipure Goat-anti-Rat, Jackson Immunoresearch Cat. No. 112-055-003), respectively.

Renaturation of SDS-PAGE gels: After regular SDS-PAGE, with or without -mercaptoethanol, the gel was renatured by incubation for 20 minutes in 40 mM Tris-HCl, pH 8.8, 1% casein, 2 mM EDTA. Thereafter the gel was overlayed with a 7.5% acrylamide chitinase activity gel containing 0.01% (w/v) glycol chitin and chitinase activity was monitored as described above.

Exochitinase and chitobiosidase activity: Soup as well as trap and leaf tissue extracts were tested for exochitinase and chitin 1,4-chitobiosidase activity using as substrates p-Nitrophenyl N-acetyl-D glucosaminide (dimer, Sigma) or p-Nitrophenyl-D-N,N-diacetyl chitobiose (trimer, Sigma), respectively. Chitinase activity was detected spectrophotometrically at 410 nm by measuring nitrophenol absorbance resulting from hydrolysis of the above substrates at pH 6.5.

FPLC analysis: The trap soup was desalted and brought to pH 10 by gel filtration on Sephadex G-25. Thereafter it was loaded onto Mono Q anion exchange column and the bound chitinase was eluted from the column with increasing concentrations of NaCl. Protein content in each fraction (1 ml) was evaluated according to the absorbance at 280 nm.

Induction of chitinase activity by chitin injection: Colloidal chitin (1 mg/100 μl), pH 5.0, was injected with a sterile syringe into a closed trap. Aliquots of the soup were collected from the closed trap at increasing intervals after injection.

Isolation of genomic DNA: Leaf tissue (1 g) was homogenized in 5 ml buffer A consisting of 1 volume of DNA extraction buffer (0.35 M sorbitol; 0.1 M Tris-HCl, pH 7.5; 5 mM EDTA and 0.02 M NaBisulfite), 1 volume nuclei lysis buffer (0.2 M Tris-HCl, pH 7.5; 50 mM EDTA; 2 M NaCl and 2% CTAB) and 0.4 vol. 5% sarkosyl. The homogenate was incubated for 20 min at 65° C. and then extracted twice with 1 volume chloroform:isoamyl alcohol (24:1). Three volumes of 6 N NaI were added to the aqueous phase and the genomic DNA was cleaned and isolated by using a High Pure filter tube from the High Pure Plasmid Isolation Kit (Boehringer Mannheim).

Isolation of total RNA: Total RNA was isolated from the lower part of the pitcher (trap) by a special hot borate/proteinase K method (Schulze et al., 1999).

Isolation of mRNA: Polyadenylated mRNA was isolated from total RNA by using oligo dT conjugated to magnetic DynaBeads (Dynal, Norway). Thereafter total cDNA was synthesized by using MM-LV reverse transcriptase (RT) and oligo(dT)₁₂₋₁₈ as a primer.

Degenerate, Inverse-PCR and gene-specific primers: Three sets of degenerate primer (#1-3) specifically designed for group 1 basic chitinase, group 2 basic chitinase and acidic chitinase, respectively, were used for the initial PCR amplification of a partial sequence of each of the chitinase genes (Table I).

TABLE I
Degenerate primers designed to amplify partial sequences
of group 1 basic, group 2 basic and acidic chitinase genes

| | |
|---|---|
| | Chitinase amino |
| Amino acid sequence type | acid sequence |

Group I Basic

| | | | |
|---|---|---|---|
| 1d | (SEQ ID NO:9) | 5′ GC/TGA/CA/GGGIAAA/GAAT/CTTT/CTAT/CAC 3′ | CEGKNFYT |
| | | | (SEQ ID NO:15) |
| 1r | (SEQ ID NO:10) | 5′ GCTG/AG/ATIGTT/AGCICCA/GAAICCT/CTG 3′ | QGFGATT/IR |
| | | | (SEQ ID NO:16) |

Group II: Basic

| | | | |
|---|---|---|---|
| 2d | (SEQ ID NO:11) | 5′ TTIGGICAA/GACIT/AG/CICAC/TGAG/AAC 3′ | F/LG/AQTSHET |
| | | | (SEQ ID NO:17) |
| 2r | (SEQ ID NO:12) | 5′ GAG/TICCICCA/GTTIATIATA/GTTT/A/C/GGT 3′ | TNIINGGI/L |
| | | | (SEQ ID NO:18) |

Group III: Acidic

| | | | |
|---|---|---|---|
| 3d | (SEQ ID NO:13) | 5′ GGGGICA/A/GAA/T/CGGIAA/T/GGA/A/GGG 3′ | WGQNGNEG |
| | | | (SEQ ID NO:19) |
| 3r | (SEQ ID NO:14) | 5′ CAIGGIGGA/G/TTA/G/TTA/G/TAA/G/AAT/C/TG 3′ | VQFYNNPPC |
| | | | (SEQ ID NO:20) |

d—direct
r—reverse

Primers #4-6 were used for the Inverse PCR strategy used for the basic chitinase genes belonging to group 2 (Table II)

TABLE II
Primers designed for the isolation
of group 2 basic chitinases by Inverse PCR strategy

| Primer sequence | | Primer Name/ |
|---|---|---|
| chitinase/Coordinates | Target | SEQ ID NO: |
| 5′ GAAAATGGACTCCGTCAGATCCTGACATTGC 3′ | Nkchit1b/1358-1388 | 4d/21 |
| 5′ GCCCCTTATTTTCTTGTCGGTGAGCATGAAC 3′ | Nkchit1b/534-564 | 4r/22 |
| 5′ CGTTTCGTTCAAGACCGCAATCTGGTTCTGGATG 3′ | Nkchit2b/1361-1394 | 5d/23 |
| 5′ CTAGTGAACTTGATGGAGTATTACTGGTAGCGGAG 3′ | Nkchit2b/454-488 | 5r/24 |
| 5′ CTACAATCAGAGGCCTTTCGGTAATGGGCTTTTG 3′ | Nkchit2b/1625-1658 | 6d/25 |
| 5′ CATCATTCCGGTGCTTGAGCATCTGATTGAACTTG 3′ | Nkchit2b/228-262 | 6r/26 |

d—direct
r—reverse

Primers #7-9 were used as gene-specific primers for the identification of the transcribed genes present in the trap secretory cells and primers #10-11 (gene-specific) were used for the isolation of the full length cDNAs (Table

TABLE III
Gene-specific pruners designed for the identification
of the transcribed genes and the isolation of the full length cDNAs

| | | SEQ ID | Target | |
|---|---|---|---|---|
| Sequence and name | | NO: | Chitinase gene | Nt coordinates |
| 5′ CGACGGTCCATATGCATGGGGATACTGTTTCAAG 3′ | 7d | 27 | Nkchit1b | 796-829 |
| 5′ CAAATGGCCACTGGGTGTAGCAGTCCAAGTTATC 3′ | 7r | 28 | Nkchit1b | 1531-1564 |
| 5′ CGTGGGGATATTGCTATCTCAG 3′ | 8d | 29 | Nkchit2b | 691-712 |
| 5′ CTACTCGGTGGCCCACAAAA 3′ | 8r | 30 | Nkchit2b | 1653-1673 |
| 5′ GTAAAACTGGACCAGACGTAGTC 3′ | 9d | 31 | Acidic | |
| | | | (partial) | |
| 5′ CGGGAATGAAGGAACCTTCAAC 3′ | 9r | 32 | Acidic | |
| | | | (partial) | |
| 5′ GTTGGGTAGTGCTTCTGCTGCTC 3′ | 10d | 33 | Nkchit1b | 68-90 |
| 5′ CATATCATCATCCACCAAATGGCCAC 3′ | 10r | 34 | Nkchit1b | 1554-1579 |
| 5′ CATCATAACGAAAATGGAGATAGCATCAG 3′ | 11d | 35 | Nkchit2b | 13-16 |
| 5′ CGGTTATTGGGCCTACTCGGTG 3′ | 11 | 36 | Nkchit2b | 1664-1685 |

d—direct
r—reverse

Primers 12-13 were specially designed primers, used for the isolation of genes by direct PCR strategy, that enable cloning of the isolated genes into a plasmid having the HA encoding sequence (Table IV).

TABLE IV
Gene-specific primers used for the isolation of genes by direct PCR strategy
(enabling cloning of the isolated genes into a plasmid
having the HA encoding sequence)

| | | SEQ ID | Target | |
|---|---|---|---|---|
| Sequence and name | | NO: | Chitinase gene | Nt coordinates |
| 5′ GAAGCTTCCATGAATGCTCCGTGCTTCTGCTTC 3′ | 12d | 37 | Nkchit1b | 1-24 |
| 5′ GCAAGCTTGTCCACCAAATGGCCACTGGGTG 3′ | 12r | 38 | Nkchit1b | 1548-1569 |
| 5′ p GAGATAGCATCAGCAAAAATATTCTTTGGTTTATCC 3′ | 13d | 39 | Nkchit2b | 4-39 |
| 5′ pGCCTCGGTGGCCCACAAAAGCCCATTAC 3′ | 13r | 40 | Nkchit2b | 1645-1670 |

d—direct
r—reverse

Fungicidal activity: The in vitro susceptibility test was adapted from the broth microdilution method NCCLS M27-P recommended by the National Committee for Clinical Laboratory Standards (NCCLS) (Espinel-Ingroff & Pfaller, 1995; ASM Manual of Clinical Microbiology). The microdilution technique involved the use of 96-well microtiter plates for cultivating the yeast in a growth medium containing sequential dilutions of the examined samples. Growth kinetics were monitored by measuring absorbance at 530 nm. C. albicans, isolate CBS 562 (originally obtained from the Central Bureau of Schimmel Cultures, Delft the Netherlands) was used as the yeast test-strain. Amphotericin B (AMB) (Squib) was used as the control antifungal drug. The minimal inhibitory concentrations (MIC) for Candida spp. are in the range of <1 to 1 μg/ml (Espinel-Ingroff et al. 1997, J.Clin. Microbiol. 35: 139). The final fungicidal test conditions were as follows: Each of the flat bottom 96 wells of the microtiter plates (Nunc) contained 0.1 ml drug (plant material or AMB) dissolved in test medium (Yeast Nitrogen Base medium supplemented with 1% glucose and 0.15% asparagine) and 0.1 ml yeast culture (0.5-2.5×10³ cell/well). The first 10 wells contained sequential two-fold (2×) dilutions of the drug and equal initial numbers of yeast cells; well 11 contained fungal control (no drug) and well 12—medium control (no drug, no fungus). Following 48 hrs incubation at 28° C., absorbance at 530 nm was measured in a microtiter reader. The minimal inhibitory concentration (MIC) value was defined as the minimal concentration of the drug that totally inhibited C. albicans propagation. Further confirmation of yeast mortality (Minimal Fungicidal Concentration) was carried out by re-plating 100 μl of the cells at the end of the drug treatment on solid medium (Sabuaruad) without the drug and counting the number of colonies after 48 hrs incubation at 28° C.

Chitinase activity assay (optical absorbance): The assay was performed according to the procedure of Tronsmo and Harman (1993) Anal. Biochem. 208:74-79). Test samples were added to flat wells of a microtiter plate. Increasing amounts (0-15 μg) of the substrate solution dissolved in 50 mM potassium phosphate buffer (pH 6.7) was added. The plates were incubated at 50° C. for 30 min. The reactions were terminated by addition of 50 μl 0.4 M Na₂CO₃ to each well, which also served to enhance the color of p-nitrophenol formed by the enzymatic cleavage of the substrate. Absorbance at 405 nm was measured with a microplate reader.

Siver staining: was done according to the method of Blum H et al. (1987) Electrophoresis 8:93-99.

Mass spectrometry: is done according to the method of Shevchenko A et al. (1996) Anal. Chem. 68:850-858.

### Example 1

### Novel Chitinases from Different Nepenthes Tissues

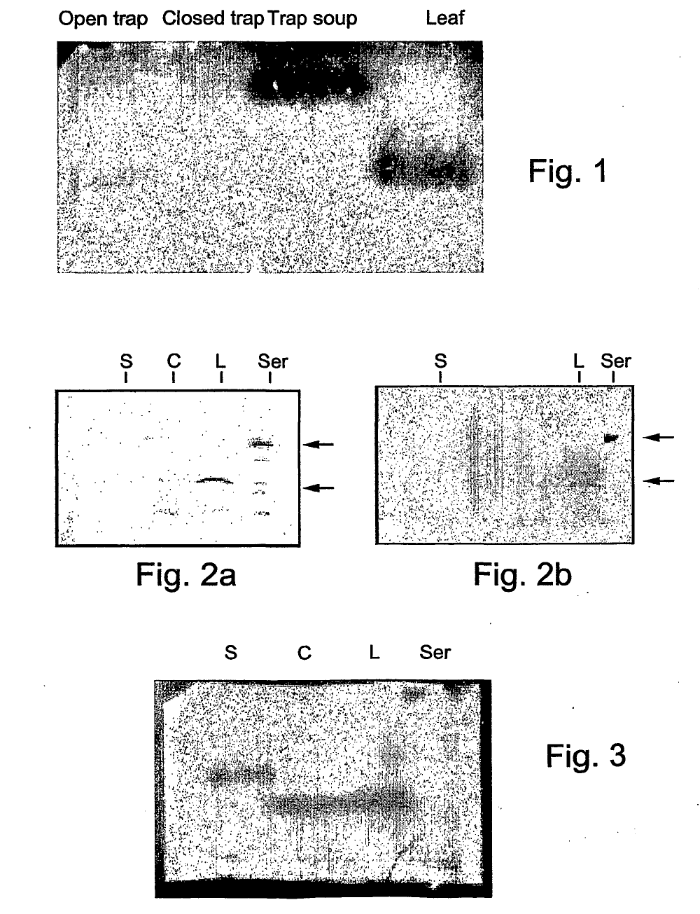

The carnivorous plant Nepenthes kassiana is a pitcher plant which uses a passive method of attraction and entrapment of the prey (Owen and Lennon, 1999). The traps are modified epiascidiate leaves, in which the adaxial surface curls around and fuses to form the inner wall of the pitcher tube. When the insects slip down the steep walls of the pitcher, they are trapped at the base in a fluid (soup) that has been reported to contain proteolytic enzymes secreted from the lower, glandular region of the pitcher, rich in secretory cells (Owen and Lennon, 1999). To characterize the chitinases present in the different tissues of Nepenthes kassiana, the mobility of the chitinases from the sterile pitcher fluid (soup), leaf tissue and pitcher (trap) tissue was studies on native polyacrylamide gels. After electrophoresis the gel was overlayed with an additional chitinase activity gel containing chitin-glycol (0.01% w/v) as a substrate. Chitinase activity was visualized after over night incubation at 37° C. as dark spots on the gel where chitinolytic activity occurred. A typical chitinase activity gel showing chitinase activity in concentrated leaf extract, trap tissue extract from closed or open traps (150 μl each) and trap soup (75 μl) is shown in FIG. 1. Surprisingly, chitinase activity is clearly demonstrated in the extracts of all three tissues. Furthermore, the soup enzyme clearly differs in its relative migration from the enzymes present both in the leaf and trap tissue.

### Example 2

### Novel Nepenthes Trap Soup Chitinase Lacks Known Chitinase Antigenic Epitopes

Western blot analyses were performed and probed with polyclonal antibodies against Serratia marcescens chitinase (ChiAII) to reveal differences in the antigenic character of the Nepenthes chitinases extracted from the various tissues. FIG. 2A shows the western blot of a 15% SDS-PAGE gel loaded with concentrates of either trap(C) or leaf tissue(L) extract (50 μl) and trap soup(S) (40 μl). Although the anti-Serratia ChiAII antibodies recognized chitinases from both the trap and leaf tissue (marked by the lower arrow), they did not recognize the soup chitinase. The gel in FIG. 2B confirms the lack of antigenic identity between trap soup and leaf (L) or trap tissue chitinases, demonstrating no immune recognition even when the protein concentration of the trap soup sample(S) was increased 22 fold, representing 875 μl initial trap soup volume.

### Example 3

### The Nepenthes Chitinases are Endochitinases

Endochitinases hydrolytically degrade chitin within the polymer. Conversely, exochitinase digestion is restricted to degradation at the termini. Evaluation of endo- versus exo-chitinase activity of Nepenthes chitinases was carried out as follows: Soup as well as trap and leaf tissue extracts were tested for exochitinase and chitin 1,4-chitobiosidase activity using the substrates p-Nitrophenyl N-acetyl-D glucosaminide (dimer) or p-Nitrophenyl-D-N,N-diacetyl chitobiose (trimer), respectively. Chitinase activity was detected spectrophotometrically at 410 nm by measuring nitrophenol absorbance resulting from the hydrolysis of the above substrates at pH 6.5. None of the Nepenthes chitinases showed any exochitinase or chitobiosidase activity, while Serratia ChiAII chitinase exhibited chitobiosidase activity. Soup, trap and leaf Nepenthes chitinases all hydrolyze glycol-chitin (FIG. 1), indicating that all three chitinases are endochitinases.

### Example 4

### Novel Nepenthes Soup, Trap and Leaf Tissue Chitinase Activity is Resistant to Partial Denaturation

Trap soup and extracts of trap and leaf tissue (without boiling) were loaded on 15% SDS-PAGE in the absence of 2-mercaptoethanol. After electrophoresis the gel was renatured by incubation in 40 mM Tris-HCl, pH 8.8, 1% casein, 2 mM EDTA and thereafter overlayed with a chitinase activity gel containing 0.01% (w/v) glycol chitin. FIG. 3 shows that semidenaturation caused by the presence of SDS during the electrophoresis stage did not inhibit chitinase activity of soup (S), trap (C) or leaf (L) tissue, when SDS was washed out after the separation. As was previously shown in non-denaturing native gels (FIG. 1), the migration rate of the soup chitinase in the semidenatured gel differs from that of the leaf and trap tissue chitinases.

### Example 5

### Novel Nepenthes Trap Soup Chitinase Activity but not Leaf Enzyme Activity is Denatured by SDS and 2-mercaptoethanol

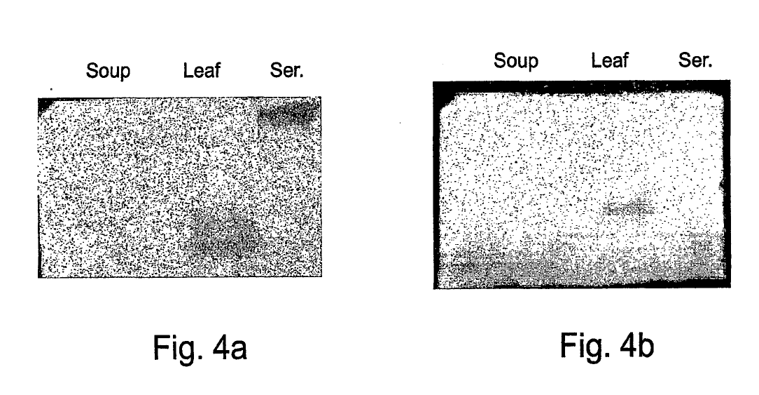

To further differentiate between Nepenthes chitinases, samples of non-boiled and boiled (5 min) soup and leaf extract were separated on 15% SDS-PAGE in the presence of both SDS and 2-mercaptoethanol. The gels were then renatured as described in Example 4 and overlayed by a chitinase activity gel. FIGS. 4A and 4B clearly demonstrate that the addition of the reducing agent 2-mercaptoethanol, in addition to SDS, completely inactivates the soup chitinase without affecting the leaf chitinase activity. In contrast, Serratia chitinase activity was abolished only in the boiled sample. Thus the novel soup chitinase clearly differs from that of the leaves. Furthermore, the importance of intact S—S bonds for the trap soup chitinase activity indicates that the trap soup chitinase is a dimer held together by inter-chain disulphide bonds (also demonstrated by its relative migration on gels displayed in FIGS. 1 and 3). To date, most active forms of plant chitinases identified are monomers of approximately 25-40 kDa. Thus the identification of a dimeric chitinase is extremely rare and unexpected.

### Example 6

### Novel Nepenthes Trap Soup Chitinase has High Specific Activity

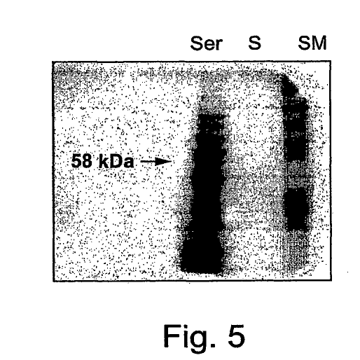

Soup chitinase is not detectable by Coomassie staining of SDS-PAGE: Since the amount of protein in the trap soup samples (75 μl) loaded on chitinase activity gels (FIG. 1) was below the detection levels of the Bradford assay, 600 μl the trap soup was concentrated and separated on 15% SDS-PAGE along with size markers and 20 μl E. coli protein extract (4 μg) containing overexpressed Serratia ChiAII (Mr 58 kDa). Protein bands were visualized by staining with Coomassie brilliant blue. Although eight fold more trap soup was applied on the Coomassie stained gel than on the activity gels (FIG. 1), no protein bands could be detected (FIG. 5, lane S). Thus, the soup chitinase has a very high specific activity.

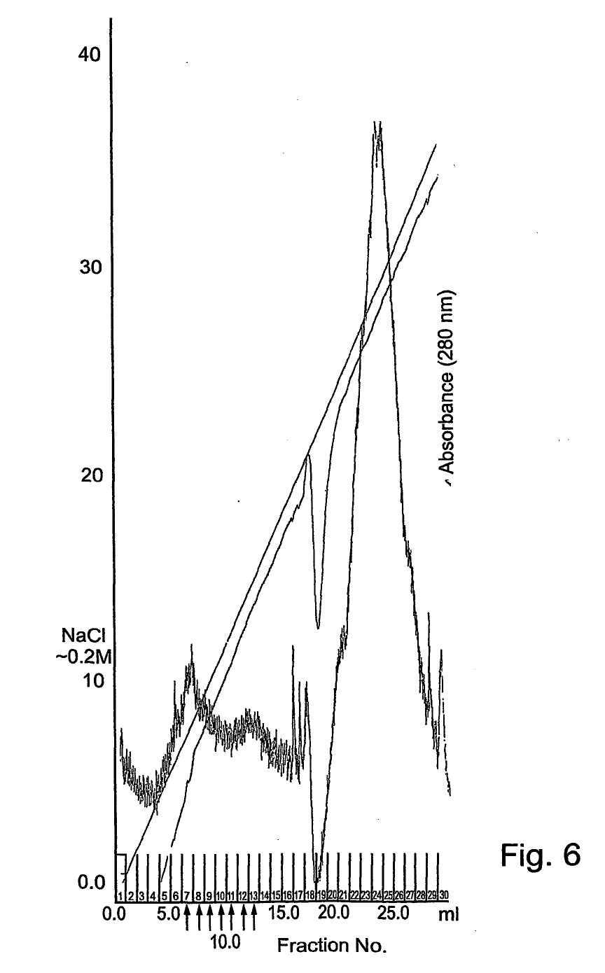

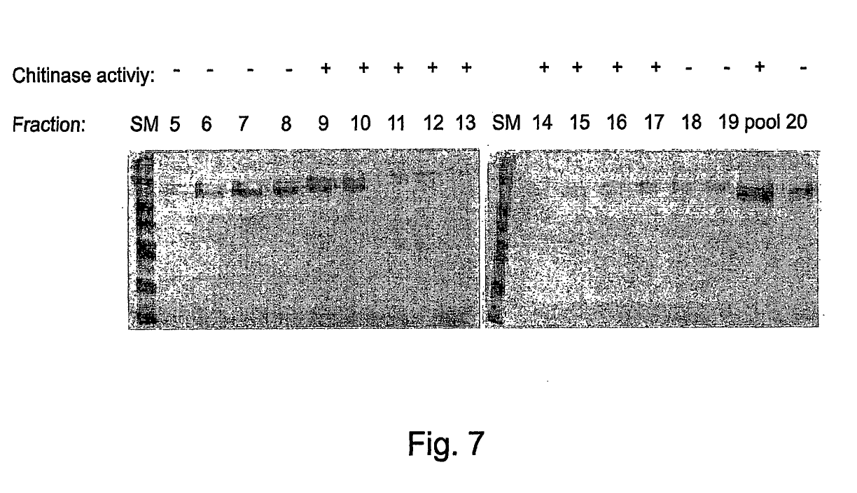

Concentration/purification of novel Nepenthes trap soup chitinase by FPLC: According to the results displayed in FIGS. 1 and 5, the trap soup chitinase possesses a very high specific activity. In order to purify and concentrate the trap soup enzyme, FPLC separation was performed using a Mono Q anion exchange column. The soup (4 ml) was first desalted and brought to pH 10 by gel filtration on Sephadex G-25. Thereafter it was applied onto the anion exchange column and the bound chitinase was eluted by washing the column with increasing concentrations of NaCl. Each (1 ml) fraction was then tested for chitinase activity on activity gels. FIG. 6 depicts a typical example of FPLC separation. Protein concentration in each fraction was evaluated according to the absorbance at 280 nm. Surprisingly, chitinase activity was detected in fractions 7 to 14 (denoted by vertical arrows), in advance of the elution of most of the OD₂₈₀ containing fractions, suggesting that the major eluted FPLC protein peak from the soup is not a chitinase. The fact that the chitinase activity could be detected already in fractions eluted at 0.2 M NaCl, suggests that the protein has a relatively high PI value. To improve the resolution of the FPLC analysis, eluted fractions (from another FPLC separation) were analyzed by SDS-PAGE and silver stained (much more sensitive than Coomassie staining). FIG. 7 clearly shows that even the two major protein bands detected in the chitinase containing fractions (lanes 9-17) do not correlate with chitinase activity, being detected in all the fractions alike. This further confirms that the novel Nepenthes soup chitinase possesses a very high specific activity.

### Example 7

### Chitin Induces Novel Nepenthes Trap Soup Chitinases

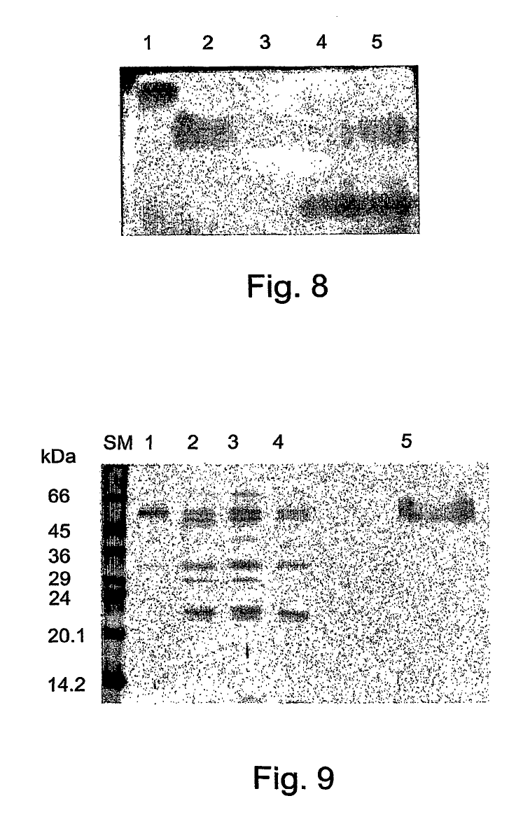

Induced chitinase activity: FIGS. 6 and 7 clearly show that under normal plant growth conditions the amount of chitinase protein produced and secreted to the closed trap fluid is very low, complicating the isolation of protein(s) displaying chitinase activity. Consequently, an attempt to increase the amount of chitinase was made by chitin injection into closed traps. Approximately 1 mg of colloidal chitin, pH 5.0, was injected into a closed trap. Chitinase activity was determined in trap soup prior to the injection, at 20 hours and at 5 days after injection. Surprisingly, injected chitin induced the appearance of at least three new chitinases migrating differently than the non-induced chitinases on native gels (FIG. 8, lanes 4 and 5).

### Example 8

### Identification of Proteins Corresponding to Chitin-Induced Novel Nepenthes Soup Chitinase Activity

Samples of the soup prior to chitin injection as well as at different times after injection were separated by 12% SDS-PAGE and visualized by silver staining. Chitin injection induced the appearance of at least four new bands and intensified two of the non-induced bands (FIG. 9, lanes 2, 3 and 4), representing both constitutive and inducible soup chitinases. With the isolation of several chitinase cDNA nucleotide sequences (from non-induced as well as induced conditions) reported hereinbelow, the amino acid sequences and their respective MW may be predicted. Five unique trap soup protein bands were excised from the SDS-PAGE gel and processed for mass spectrometric sequencing.

### Example 9

### Antifungal Effect of Trap Soup

Chitinases are known to play a major role in plant defense response by hydrolyzing chitin containing fungal cell walls. This hydrolytic activity retards fungal growth and delays or avoids the invasion of pathogens into plant tissues. Antifungal activity of Nepenthes trap soup was studied by three different in vitro bioassays.

Chitin-induced novel Nepenthes trap soup chitinase inhibits in vitro growth of the human pathogen Candida albicans: In order to determine the antifungal effects of novel Nepenthes trap soup chitinase on the important human pathogen C. albicans, sterile Nepenthes kassiana trap soup from normal and chitin-induced traps was collected and concentrated. For comparison, leaf extracts of three other carnivorous plants (Dionea, Drosera and Sarracenia) were prepared in the presence of protease inhibitors. Antifungal lethal/inhibitory activity of samples of the different extracts were evaluated in vitro using a Candida albicans growth assay, as detailed in Methods. The results, expressed as minimal inhibitory concentration (MIC) of each sample, are presented in Table V.

TABLE V
Inhibition of Candida albicans growth by trap soup from
carnivorous plants

| | | Chitin | MIC [original |
|---|---|---|---|
| Tested Samples | Total Protein Conc. | induction | sample dilution] |
| Nepenthes ssp. | 1.2 mg/ml | − | undetectable |
| secreted extract | | | |
| Nepenthes ssp. | 3.1 mg/ml | + | ⅛ |
| secreted extract | | | |
| Nepenthes ssp. | 3.1 mg/ml | + | ½ |
| secreted extract + | | | |
| chitin | | | |
| Dionea sp. secreted | 19.8 mg/ml | − | 1/32* |
| extract | | | |
| Drosera sp. secreted | 8.4 mg/ml | − | 1/32* |
| extract | | | |
| Sarracenia sp. | 16.4 mg/ml | − | ½ |
| secreted extract | | | |
| Serratia marcescens | 2 units/ml | − | undetectable |
| recombinant | | | |

*-maximal sample dilution used in the experiment

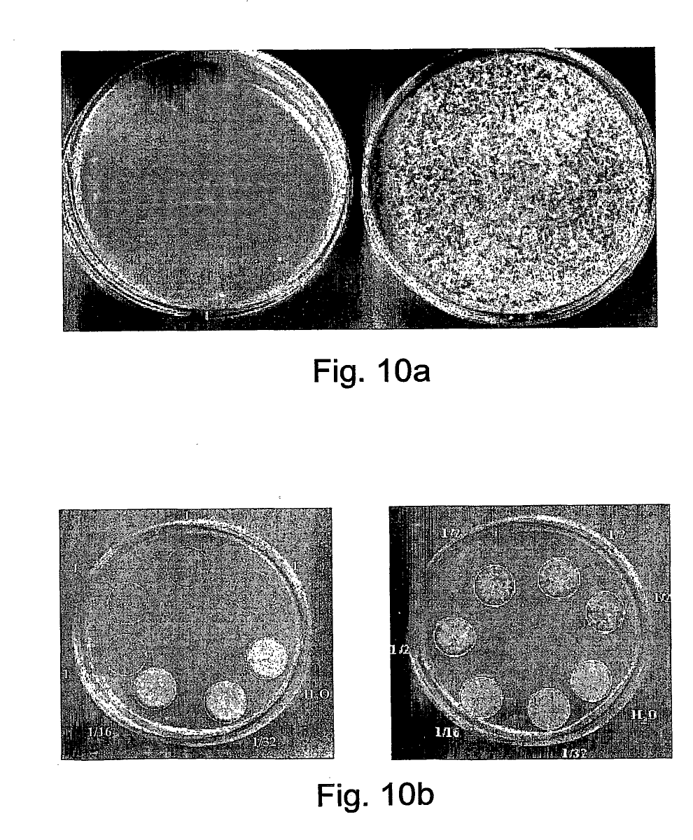

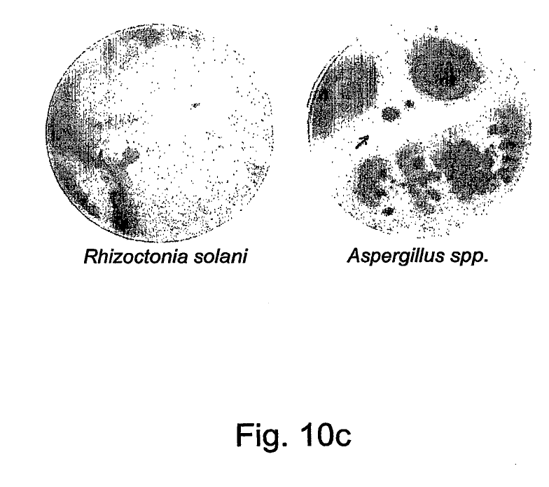

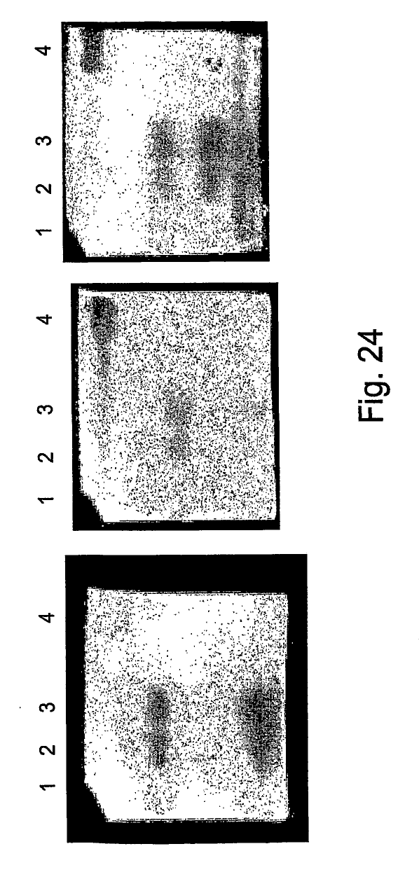

These results indicate that while normal trap soup had no effect, the chitin-induced trap soup was very efficient (⅛th dilution of the sample) in inhibiting growth of Candida albicans. When the injected chitin was present in the assayed sample, the MIC increased. This indicates that chitinase, indeed, plays a crucial role in the antifungal activity. Moreover, when the minimal fungicidal concentration (MFC) for the chitin-induced soup was determined, it was found to be at ¼ the dilution of the sample (FIG. 10a), indicating extensive disruption of the C. albicans at that concentration. Furthermore, while commercial Serratia marcescens chitinase had no inhibitory effect, all three additional carnivorous plants leaf extracts were very potent in inhibiting Candida albicans growth (Table V, see also FIG. 24).

### Example 10

### Chitin Induced Novel Nepenthes Trap Soup Chitinase Inhibits Growth of Septoria tritici

The effect of chitin induced soup on growth of the plant pathogen Septoria tritici was determined by culturing conidia with increasing dilutions of chitin-induced Nepenthes trap soup, and measuring OD₂₈₀, as detailed in Methods. The chitin induced soup, at the dilution of ½, significantly inhibited the growth of Septoria triciti (FIG. 10b). As in the abovementioned C. albicans assay, higher concentrations (undiluted) of trap soup were fungicidal in effect (FIG. 10b).

### Example 11

### Novel Nepenthes Trap Soup Chitinase Inhibits the Development of Mycelia of Rhizoctonia solani and Aspergillus spp.

The antifungal effect of Nepenthes trap soup on the growth of mycelia in the common plant pathogens R. solani and Aspergillus was estimated by applying a 20 μl drop of 5 fold concentrated trap soup on a plate containing log phase culture of either Rhizoctonia or Aspergillus. FIG. 10c (see arrow) shows that the trap soup chitinase inhibits the development of mycelia of Rhizoctonia solani and Aspergillus sp. Thus, the novel Nepenthes trap soup chitinase demonstrated significant growth inhibitory and fungicidal activity on all of the species of fungi tested.

### Example 12

### Isolation of Partial Genomic Sequences of Nepenthes Chitinases by PCR Using Degenerate Primers

According to the present data in the Gene Bank (NCBI), plant chitinases are grouped into three types based on their amino acid sequence: basic chitinases (group 1), basic chitinases (group 2)- and acidic chitinases (group 3). In order to isolate the genes for the novel chitinases of the present invention, a set of degenerate primers (see Table I in Methods) was designed for each group and used leaf total DNA as a template for PCR screening. Three partial chitinase genes were isolated. Two of the DNA fragments (895 bp and 1.1 kb) were cloned and sequenced and both showed homology to group 2 of basic chitinase genes, while the third (536 bp) showed homology to the acidic chitinases. Since our biochemical characterization of the novel Nepenthes trap soup chitinase (high chitinase specific activity and high PI value) suggested that it belongs to class I basic chitinases, genomic and cDNA sequences belonging to the basic chitinase group were isolated.

### Example 13

### Isolation and Complete Nucleotide Sequences of Two Novel Basic Nepenthes Chitinase Genes

Based on the isolated partial sequences, a set of primers (Table II) was synthesized for each gene to further isolate the rest of the two types of basic chitinase genes by Inverse PCR strategy. The PCR reactions were repeated with new sets of inverse primers (Table II) until the entire genes were isolated and their sequences confirmed.

TABLE VI
Primers designed for the isolation of group 2 basic chitinases
by Inverse PCR strategy

| | | SEQ ID | Target | |
|---|---|---|---|---|
| Sequence and name | | NO: | Chitinase gene | Nt coordinates |
| 5′GAAAATGGACTCCGTCAGATCCTGACATTGC3′ | 15d | 41 | Nkchit1b | 1358-1388 |
| 5′GCCCCTTATTTTCTTGTCGGTGAGCATGAAC3′ | 16r | 42 | Nkchit1b | 534-564 |
| 5′CGTTTCGTTCAAGACCGCAATCTGGTTCTGGATG3′ | 17d | 43 | Nkchit2b | 1361-1394 |
| 5′CTAGTGAACTTGATGGAGTATTACTGGTAGCGGAG3′ | 18r | 44 | Nkchit12b | 454-488 |
| 5′CTACAATCAGAGGCCTTTCGGTAATGGGCTTTTG3′ | 19d | 45 | Nkchit12b | 1625-1658 |
| 5′CATCATTCCGGTGCTTGAGCATCTGATTGAACTTG3′ | 20r | 46 | Nkchit12b | 228-262 |

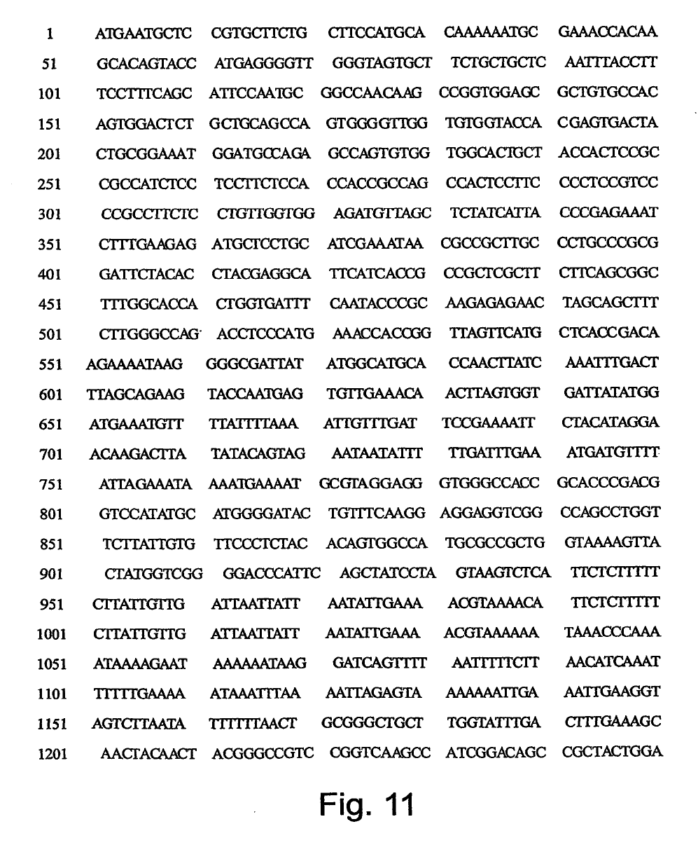

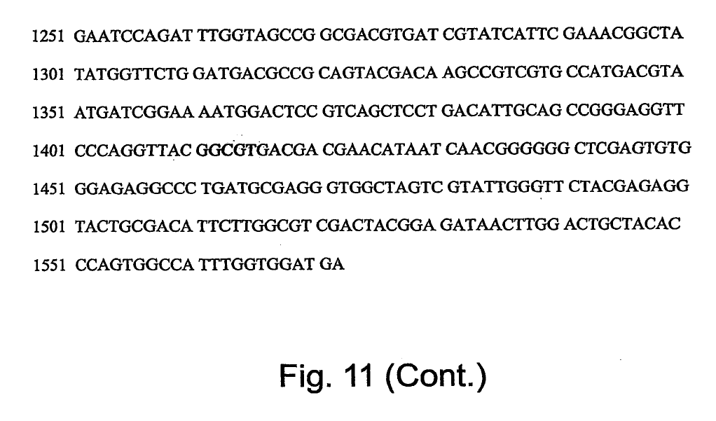

Sequencing and amplification of Nepenthes chitinase 1 genes reveals multiple copies of Chitinase 1 gene: FIG. 11 shows the complete sequence of the basic chitinase 1 gene isolated by Inverse PCR strategy and termed Nkchit1b (SEQ ID NO:1). The entire length of the gene from the putative translation start codon to the stop codon is 1572 bp. Sequence alignment with other chitinases in the genebank and consensus splicing sites suggested the presence of two putative introns of sizes 247 bp and 269 bp. The splicing sites were later verified by RT-PCR strategy, using 5′ and 3′ gene-specific primers (see Table IV in Methods) designed to amplify the fill length cDNA sequence of the gene using mRNA from chitin induced traps as template for the reverse transcriptase.

Another chitinase 1 encoding gene, isolated by direct PCR strategy using genomic DNA as template and specifically designed primers (Table IV), was termed Nkchit1b-gI (SEQ ID NO: 48). The two distinct chitinase 1 genes Nkchit1b and Nkchit1b-gI) have identical exons but different introns. Nkchit1b-gI was used for plant transformation.

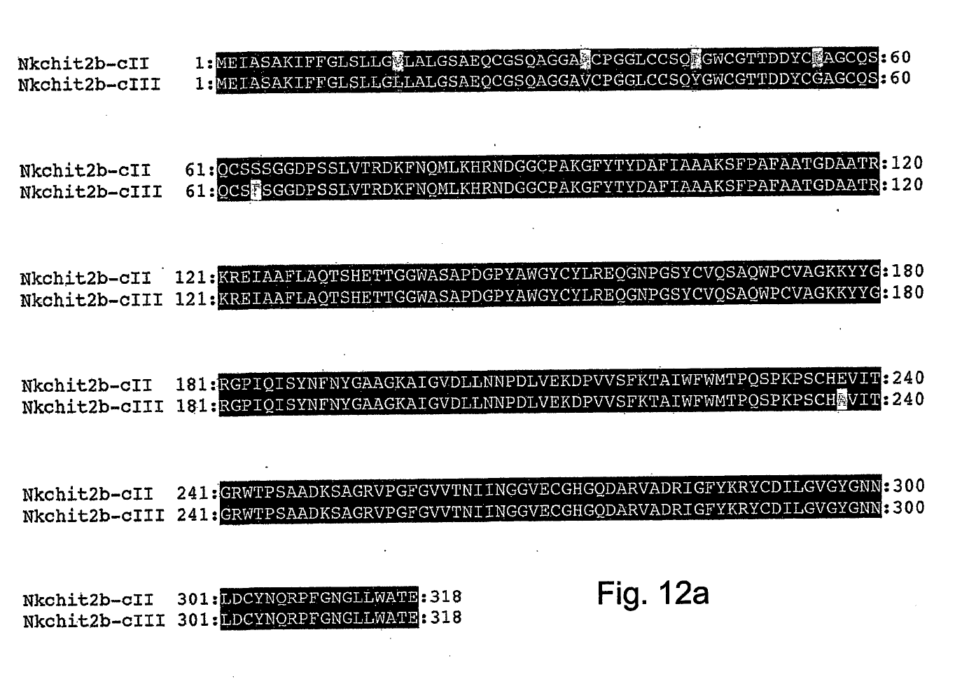

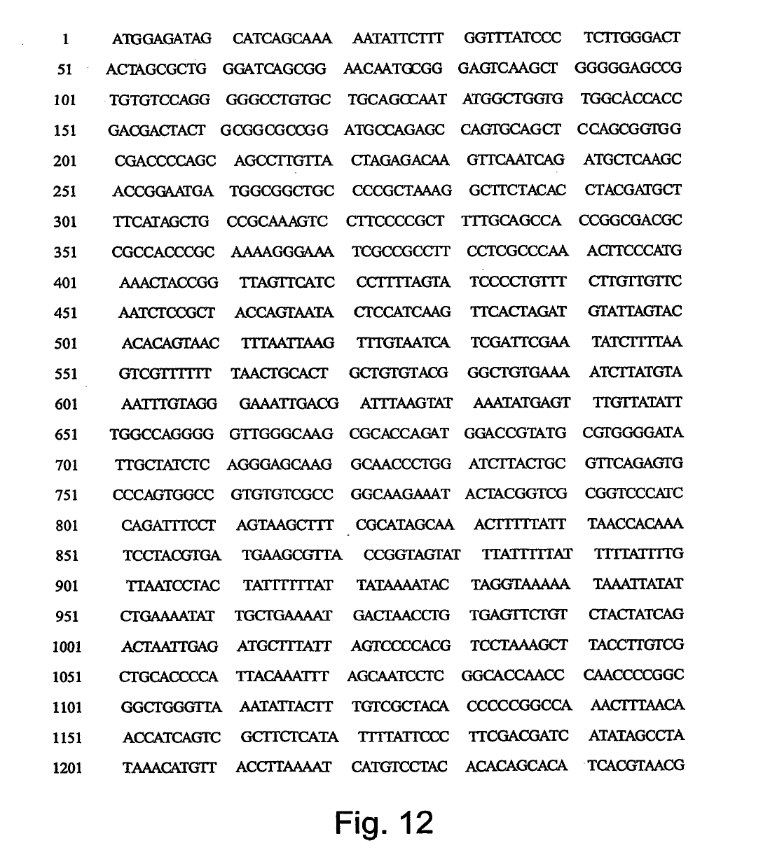

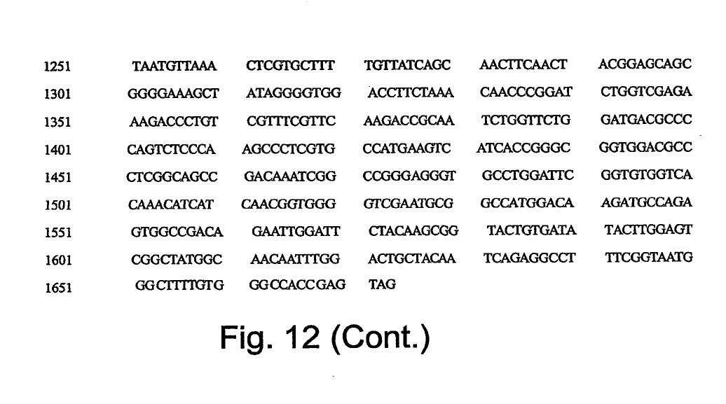

Sequencing and amplification of Nepenthes chitinase 2 genes reveals multiple copies of Chitinase 2 gene with amino acid substitutions: FIG. 12b shows the complete sequence of the basic chitinase 2 gene isolated by Inverse PCR strategy and termed Nkchit2b (SEQ ID NO:2). The entire length of the gene from the putative translation start codon to the stop codon is 1673 bp and the alignment with other chitinases in the gene bank and consensus splicing sites suggested the presence of two putative introns (248 bp and 468 bp), that were later verified based on the respective cDNA sequences (similarly as detailed above for Nkchit1b).

Direct PCR strategy using genomic DNA as template and specifically designed primers (Table V) revealed an additional gene encoding chitinase 2, termed Nkchit2b-gII, that has exons differing in codons of four amino acids from those of Nkchit2b. In order to determine which genes are expressed, polyadenylated mRNA was isolated, as described in Materials and Methods, from secretory cells of the traps. RT-PCR enabled the isolation of two cDNA types, termed Nkchit2b-cII and Nkchit2b-cIII, encoding chitinase 2 type enzymes (Table III). The Nkchit2b-cII cDNA corresponds to the genomic sequence Nkchit2b-gII while the chitinase 2 encoded by Nkchit2b-cIII differs in six amino acids from that of Nkchit2b-cII. A fourth type of chitinase 2 encoding gene was isolated from a cosmid library, but since no corresponding cDNA was found, only the two expressed genes were used for further study. FIG. 12a shows the alignment of the two deduced amino acid sequences of the chitinase 2 cDNAs. Thus, it was surprisingly demonstrated that the novel Nepenthes chitinase genes belong to a multigene family.

### Example 14

### Amino Acid Sequence Non Homology in Novel Nepenthes kassiana Chitinases and Functional Implications Thereof

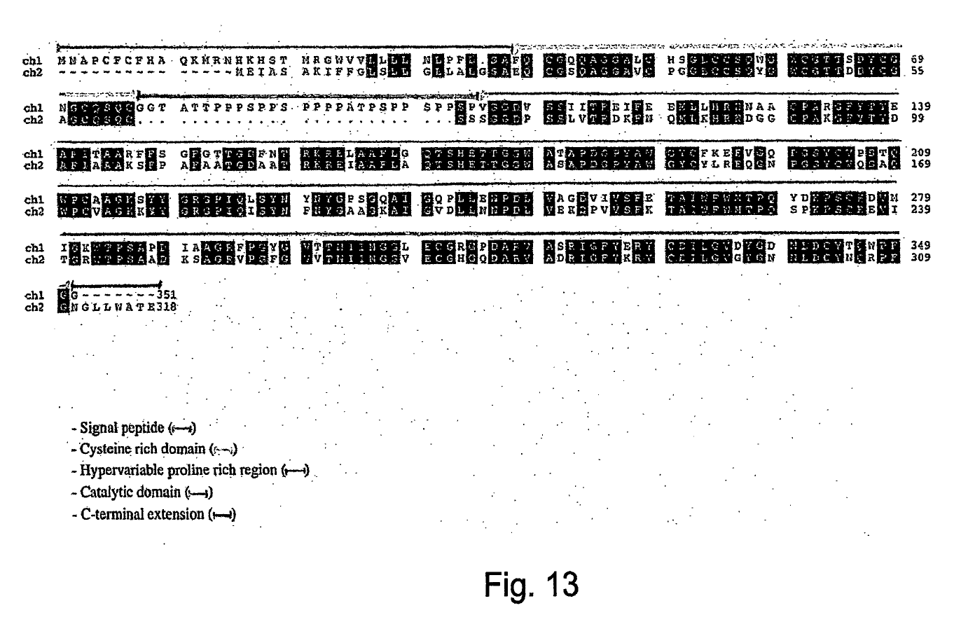

The deduced amino acid sequences of the two Nepenthes chitinase genes, (based on the cDNA sequence) were termed NkCHIT1b (SEQ ID NO:5) and NkCHIT2b (SEQ ID NO:6). A BLAST search for amino acid sequence homology of NkCHIT1b showed highest (67%) identity and 76% homology to Oryza sativa (L37289) chitinase (and 67% identity at the DNA level) while NkCHIT2b showed 73% identity and 78% homology to basic endochitinase precursor of Vitis vinifera (P51613) (and 75% identity at the DNA level). FIG. 13 shows the amino acid sequence alignment (prettybox) of NkCHIT1b and NkCHIT2b. They have 75% similarity and 70% identity as determined by the GAP program of GCG.

Class I basic chitinases are usually composed of five structural domains: 1) N-terminal signal peptide 2) cysteine rich domain 3) proline rich hinge region 4) catalytic domain and 5) carboxy-terminal extension. As illustrated in FIG. 13, both Nepenthes chitinases have signal peptides, although different in length and amino acid composition; a cysteine rich domain and a catalytic domain. However, a proline rich hinge region is present only in NkCHIT1b. The length of the hinge region is known to vary among different chitinases and may be absent altogether, as in NkCHIT2b. A carboxy-terminal extension, rich in hydrophobic amino acids, is suggested to be a signal for vacuolar targeting (Graham, 1994). In case of tobacco chitinases, for example, deletion of the NLLVDTM amino acid consensus sequence at the C-terminal end led to the secretion of the modified chitinase to the apoplast (Chrispeels and Raikhel, 1992). Comparison of the C-terminal parts of the two Nepenthes kassiana basic chitinases revealed the presence of an eight amino acid long hydrophobic extension only in NkCHIT2b.

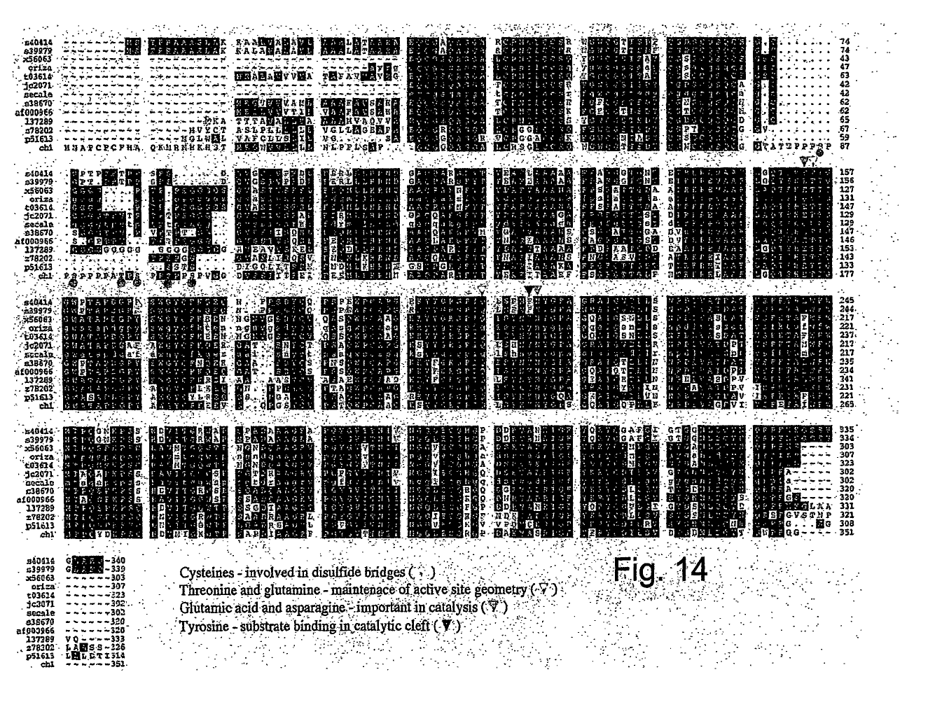

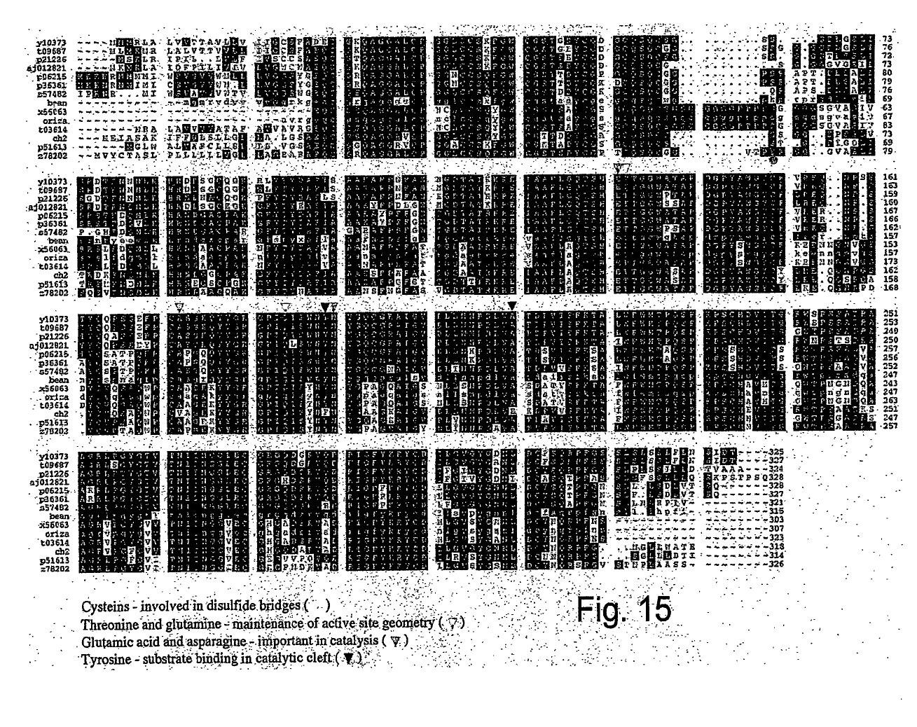

FIGS. 14 and 15 show a multiple sequence alignment of each of the two Nepenthes chitinases with protein database-derived monocot and dicot chitinase sequences displaying the highest amino acid sequence homology to the novel chitinases. Conserved amino acid residues suggested to be of functional importance (Graham, 1994; Hamel et al., 1997) are indicated by different colors. Thus, the eight cysteines (C), present at identical positions (yellow arrow) in the chitin-binding region of most of the sequences, are involved in the formation of disulfide bridges allowing the correct folding of the domain into a compact active conformation. The threonine (T) and glutamine (Q), that play a role in the active site geometry (red arrow) as well as the glutamic acid (E) and asparagine (N) that have been shown to be important for catalysis (green arrow) (Graham, 1994; Hamel et al., 1997), are all present also in both novel Nepenthes chitinases (FIGS. 14&15). However, the conserved tyrosine (Y), thought to bind the substrate in the catalytic cleft (Hart et al., 1995), is altered to phenylalanine (F) in NkCHIT2b (blue arrow, FIG. 15). Two other prominent differences distinguishing NkCHIT2b from NkCHIT1b, as well as from other chitinases are valine (brown arrow) and glutamic acid (black arrow) of NkCHIT2b in place of alanine (FIG. 15). Thus, although the novel Nepenthes chitinases possess regions of amino acid sequence homology with chitinases of other species, the are clearly unique in the composition of the catalytic cleft.

### Example 15

### Three-Dimensional Modeling of NkCHIT2b Confirms Unique Structure in Catalytic Cleft

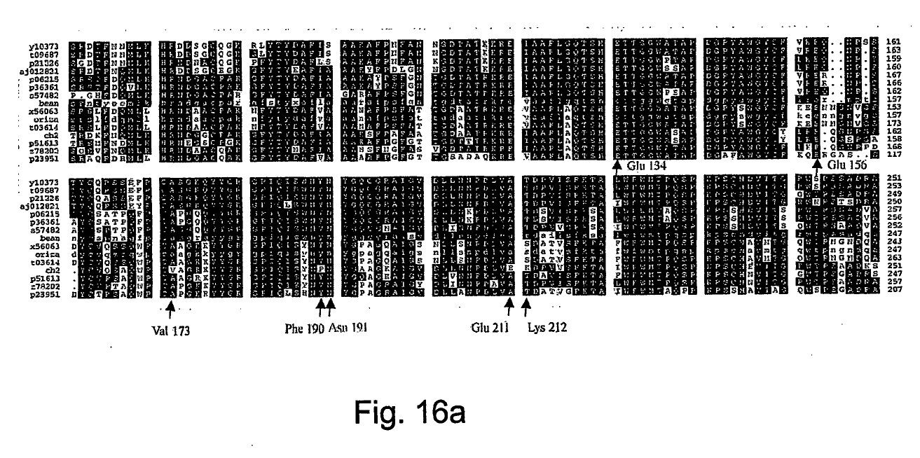

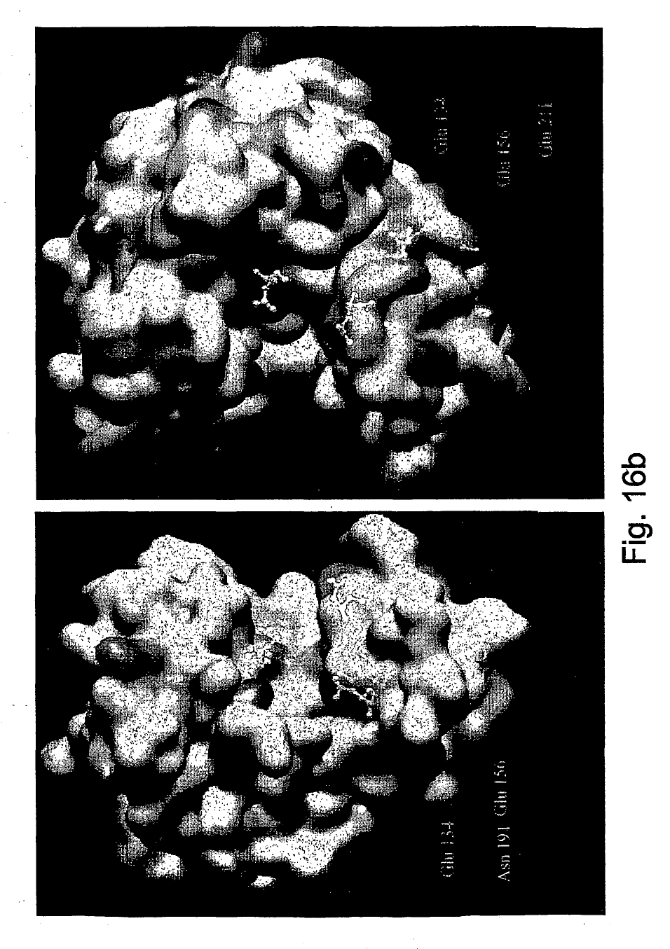

To study the possible effect of the abovementioned unique amino acid sequences, a three-dimensional structural modeling (SWISS-MODEL Protein Modeling, http://www.expasy.ch/swissmod/SWISS-MODEL) of NkCHIT2b (which appears to be the constituent chitinase in the trap soup) was performed. FIGS. 16A and 16B summarize this information. The prediction is based on the crystal structure of endochitinase from Hordeum vulgare L. (barley) seeds (Song et al., 1993; PDB and Swissprot accession numbers are 1CNS and p23951, respectively). FIG. 16A shows a segment of a multiple sequence alignment of NkCHIT2b, with the sequences from the gene bank that show closest homology to NkCHIT2b, into which the sequence of barley chitinase was included. In addition to the above mentioned altered amino acid residues, two NkCHIT2b glutamic acids (#134 and 156) were marked, which have been shown to be essential for the catalytic activity in barley chitinase, as well as the asparagine #191 which in barley is located in the substrate binding cleft (Andersen et al., 1997). Mutation of either of these glutamic acids (Glu 67 and Glu 89 in barley chitinase, which correspond to Glu 134 and Glu 156 in NkCHIT2b) results in a substantial loss of barley chitinase activity. Similarly, the asparagine (Asn 124 in barley which corresponds to Asn 191 in NkCHIT2b), was shown to play an important functional role (Andersen et al., 1997).

FIG. 16A shows the model of NkCHIT2b when viewed from left and right side of the same molecule. The locations of the relevant amino acid residues are marked on the molecule and they are specified at the left and right side of the molecule according to their relative location. It can be seen that the Glu 134 and 156 and Asn 191, that have been shown to be crucial for catalytic activity in barley chitinase, are located in the catalytic cleft. It is interesting to note that the hydrophobic amino acid Phe 190 of NkCHIT2b replaces the highly conserved polar tyrosine located in the catalytic cleft, adjacent to the Asn 191, which in barley has an important functional role. Moreover, Glu 211 and Lys 212, which both are charged amino acids, replace hydrophobic (alanine) and polar (threonine) amino acids in the corresponding locations in barley. Such changes in charge in the vicinity of the catalytic cleft, could account for changes in catalytic activity unique to the novel Nepenthes chitinase.

### Example 16

### Post-Translational Modifications in Novel Nepenthes Chitinases: O-glycosylation Sites in NkCHIT1b and NkCHIT2b

To further distinguish between Nepenthes and other plant chitinases possible O-glycosylation sites in NkCHIT1b and NkCHIT2b were predicted. Although some plant chitinases have been shown to be glycoproteins (Margis-Pinhiero et al., 1991, De Jong et al., 1992), the majority are not (Graham, 1994).

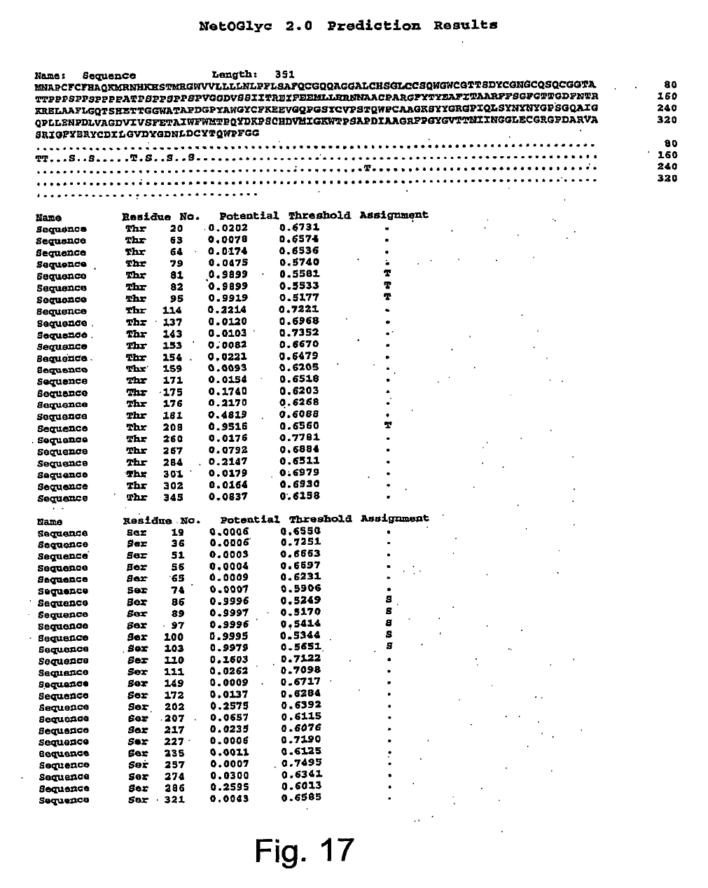

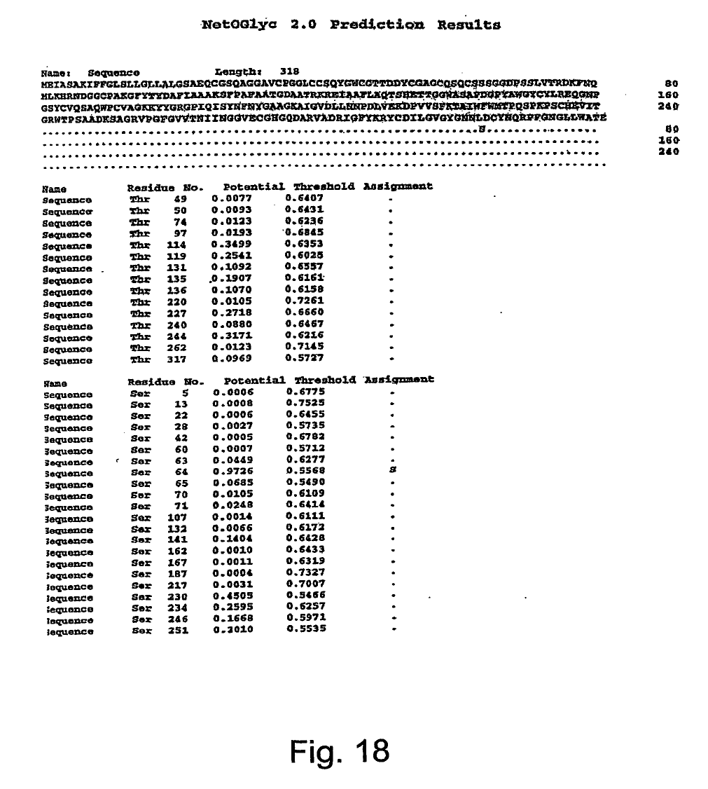

The predictions were performed with the ExPaSy Molecular Server (NetOGlyc prediction, http://www.cbs.dtu.dk/services/NetOGlyc) which predicts post-translational modifications. Surprisingly, a number of glycosylation sites were identified. FIGS. 17 and 18 show that while NkCHIT1b has nine possible glycosylation sites, only one possible site was predicted for NkCHIT2b (see also FIGS. 14 and 15, violet dots). The nine sites (NkCHIT1b) were all concentrated in the proline rich hinge region. Thus, these unexpected post-translational modifications may be important to the character and high specific activity of the novel Nepenthes chitinase.

### Example 17

### Expression of Chitinase(s) Genes in Nepenthes Trap Secretory Cells

The Nepenthes trap soup chitinase displays a very high chitinase activity. However, the trap soup is not active in protein synthesis. Thus, it was important to identify and isolate the novel Nepenthes chitinase mRNA from the trap secretory region (bottom part of the pitcher) responsible for the synthesis of the secreted chitinases.

Nkchit2b is preferentially expressed in non-induced traps: Total RNA was isolated from the bottom part of traps by a special hot borate/proteinase K method (Schulze et al., 1999). mRNA was isolated from total RNA by using oligo dT conjugated to magnetic DynaBeads (Dynal, Norway) and thereafter total cDNA was synthesized by using MM-LV reverse transcriptase (RT) and oligo(dT)₁₂₋₁₈ as a primer. In order to differentiate between the three different (two complete basic and one partial acidic) Nepenthes chitinases isolated so far, a set of gene-specific primers for each gene was synthesized (#7-9, Table IV) as described in Methods. These primers were then used to determine which of the three genes are most actively transcribed in the trap secretory cells under varied conditions.

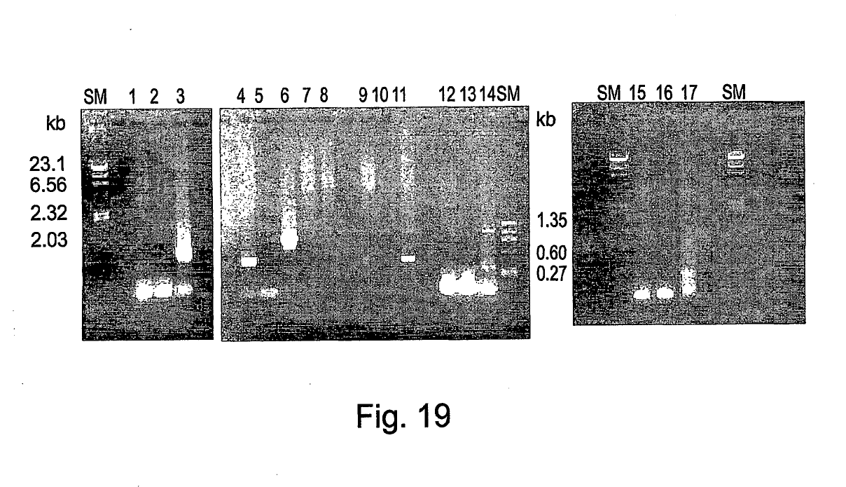

FIG. 19 shows the RT-PCR results using the three sets of primers (Nkchit1b, Nkchit2b and acidic chitinase) as well as two sets of basic chitinase degenerate primers (#1-2, Table I) previously used for the isolation of basic chitinase genes. It can clearly be seen that out of the three chitinases studied thus far, Nkchit2b comprises the major chitinase transcript present in the mRNA of trap secretory region. Amplification with Nkchit1b or acidic chitinase primers yielded only very faint bands suggesting that their transcript levels in the trap tissue is very low. PCR amplification with degenerate primers using the same cDNA preparation gave no distinct bands. When genomic DNA was used as a template instead of cDNA, longer transcription products were detected (lanes 3 and 6 compared to 1 and 4, respectively). This confirmed that in both basic genes there is an intron localized in the amplified region between each set of primers exactly matching the intron size.

Alternate expression of novel Nepenthes trap soup chitinases in chitin-induced traps: The abovementioned Examples demonstrate that chitin injection into closed traps induces the appearance of at least three new soup chitinases. In order to further characterize the chitinase profile of induced versus uninduced traps, products of the RT-PCR analysis with cDNA from induced and uninduced traps were used as templates for amplification. FIG. 20 shows that there is a clear increase in the amount of Nkchit1b transcript in the induced traps, while the Nkchit2b amount does not change significantly. Furthermore, an additional chitinase product appeared in the induced traps when a set of degenerate primers (group2) was used. This 435 bp band was isolated, cloned and sequenced and it is identical to Nkchit2b-cIII. Thus, the transcript of Nkchit2b-cIII is also chitin inducible.

### Example 18

### Expression of Novel Nepenthes Chitinases Encoded by Nkchit1b-gI, Nkchit2b-gII and Nkchit2b-cIII in Transgenic Tobacco Plants and Suspension Cultures

It has been demonstrated above that only very low levels of chitinase protein are present in the trap soup (see Example 6), constituting a considerable obstacle to the enzyme's purification and production. Furthermore, current uses of biocidal compounds in agricultural and clinical applications require compatibility with developing DNA and cloning technologies. Thus, methods of transgenic expression and purification of the chitinases are of great interest. However, it is well known that accurate expression and retention of biological activity of chimeric cloned proteins often requires extensive experimentation and genetic manipulation. To this end, the novel Nepenthes chitinase genes of interest were translationally fused at the 3′ terminus to a nine amino acid long HA peptide tag (Ferrando et al., 2001). The HA peptide enables purification of significant amounts each chitinase and the subsequent determination of the kinetic properties of each enzyme.

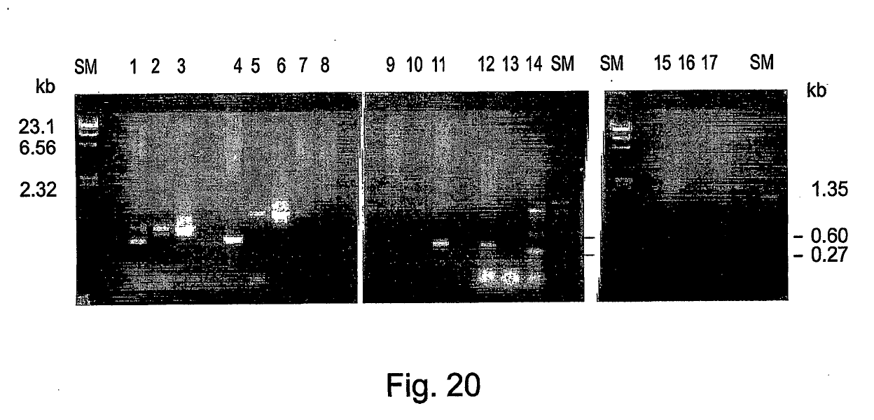

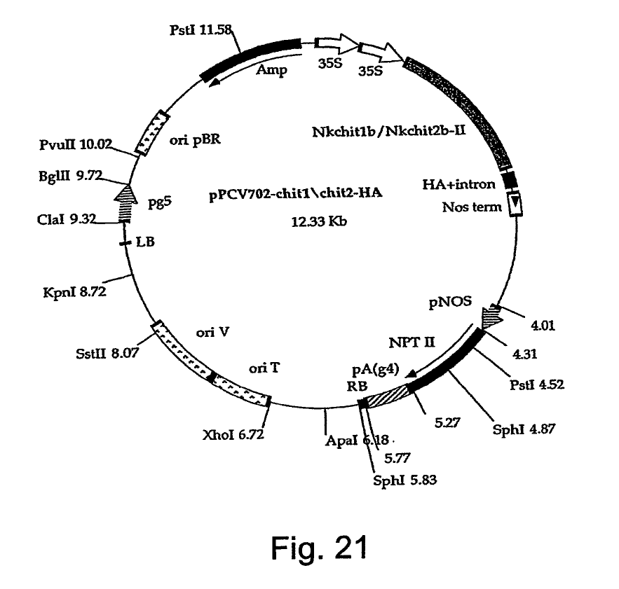

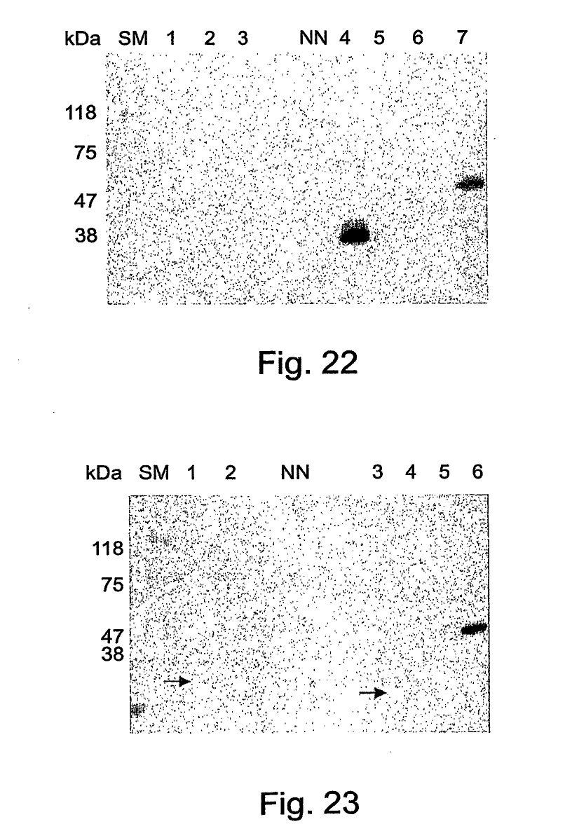

Nkchit1b-gI, Nkchit2b-gII and Nkchit2b-cIII were synthesized by direct PCR strategy using specific proof reading Taq polymerase and specially designed primers (Table IV) that enable further cloning of the genes into a plasmid carrying a plant expression cassette with the HA encoding sequence. Thereafter, the chitinase-HA cassette was cloned into the pPCV702 binary vector [Koncz. et al. (1989) Proc. Natl. Acad. Sci. USA 86:8467-8471] for subsequent Agrobacterium-mediated transformation of tobacco leaf discs. FIG. 21 shows the final pPCV702 vector which in addition to the nptII selectable marker carries either the Nkchit1b-gI, Nkchit2b-gII or Nkchit2b-cIII gene fused to the sequence encoding HA epitope at the 3′ end and driven by the constitutive CaMV 35S promoter. After co-cultivation of leaf discs with the engineered Agrobacterium, shoot regeneration was induced in the presence of kanamycin and hormones. Kanamycin resistant plants obtained from the transformations were screened for the expression of each of the chitinase-HA fused proteins by Western analysis using anti-HA antibodies. FIGS. 22 and 23 show typical Western blot analyses of the kanamycin resistant plants. Wild type tobacco (NN) and transgenic tobacco expressing Serratia chitinase fused to the HA tag (MW˜59,000 Dalton) were used as negative and positive controls, respectively. The anticipated sizes of chitinase1 and chitinase2 protein fused to the Ha tag are 36,000 and 32,700 Dalton, respectively. Four of the six plants screened expressed the chitinase1 enzyme (FIG. 22). The observed molecular weight was as expected, indicating accurate processing of the Nepenthes chitinase in the tobacco plants. Plant #4 showed a relatively high amount of chitinase1-HA product, indicating that this transgenic plant contains several copies of introduced chitinase1 transgene. Thus, plant #4 is a good candidate for subsequent purification of the chitinase1 protein. In FIG. 23 demonstrates the expression of the chitinase2 enzyme in two of the five plants screened. The observed molecular weight of the chitinase2-HA protein was 32,700 Daltons, indicating accurate intron splicing in these transgenic plants as well.

Both Nepenthes chitinases possess a leader peptide that targets the protein into the endoplasmic reticulum. Only chitinase2, however, has the carboxy-terminal extension (CTE) which targets the protein into vacuoles. Chitinase1, devoid of the CTE is thus expected to be secreted to the extracellular space (Legrand et al., 1987; Swegle et al., 1992; Vad et al., 1991). Thus, transgenic plants accurately expressed novel Nepenthes chitinases retaining both their physical and enzymatic integrity.

### Example 19

### Novel Chitinase Activity in Additional Carnivorous Plants

The abovementioned Examples indicate that Nepenthes kassiana (Nepenthaceae) possesses a group of highly active, novel chitinases. Although such high chitinase activity has not been demonstrated in other species, three additional genera (Dionea sp., Drosera sp., and Sarracenia sp.) of carnivorous plants belonging to two separate families (the first two belong to Droseraceae and the third to Sarraceniaceae) were screened. The three representatives were screened for antifungal properties as well as chitinase activity. These three carnivorous plants have developed individual structural mechanisms for trapping insect prey: whereas Nepenthes and Sarracenia use trap soup to digest the insects, the other plants have either sticky droplets that trap the prey or leaves that fold around the prey. Therefore, both the antifungal as well as the chitinase activity assays were performed with trap tissue extracts rather than trap soup. Table Ia (see Example 9, above) summarizes the results of the antifungal activity of the tissue extracts on the human pathogen Candida albicans. Both Dionea and Drosera extracts demonstrate very potent inhibition of Candida albicans growth, effective even at the lowest examined dilution (1/32). Sarracenia is also active in inhibiting the growth of Candida albicans, albeit at higher concentrations. Taken together these results demonstrate, for the first time, a novel fungicidal effect of carnivorous plant extract on the human pathogen Candida albicans.

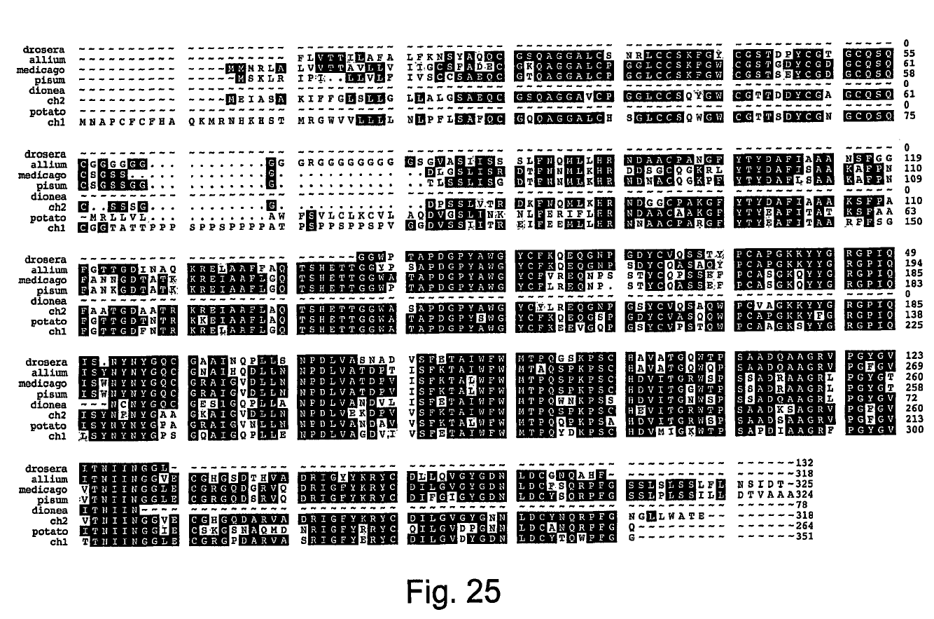

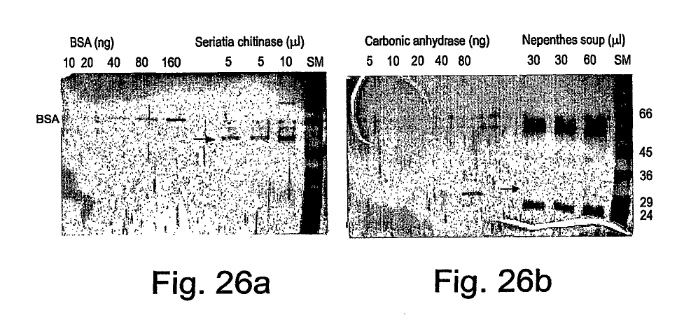

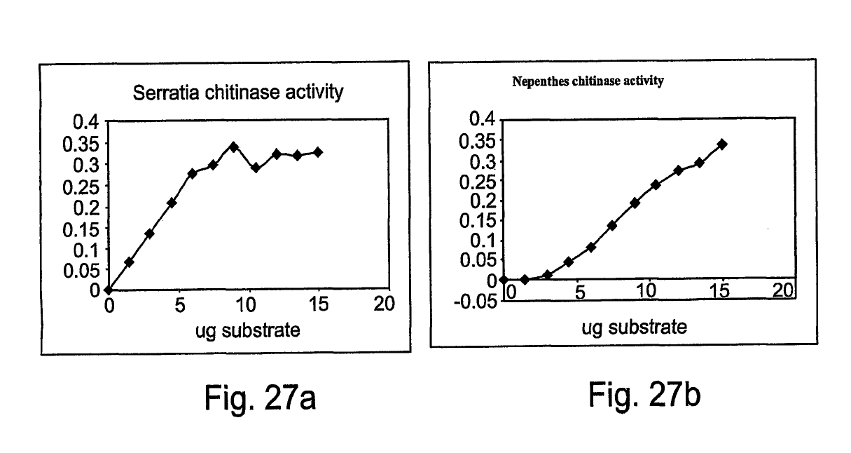

FIG. 24 summarizes the results of chitinase activity in the three different carnivorous plants. Strong chitinase activity bands were detected in all three plants. Two different chitinases, according to the migration distance, were present in Drosera spathulata. These results indicate that the three diverse types of carnivorous plants have multiple forms of active chitinases. In order to compare the relative activities with chitinases from other plants the chitinase genes from Drosera and Dionea were isolated. FIG. 25 shows the alignment of the deduced amino acid sequences of two partial chitinase genes, one from Drosera (SEQ ID NO:7) and another from Dionea (SEQ ID NO:8), with plant chitinases revealing closest homology in the gene bank (BLAST search). Drosera chitinase shows the closest homology to Allium sativum and Solanum tuberosum (81% and 76% identity, respectively) and Dionea to Medicago trucatula and Pisum sativuin (76% and 75%, respectively). The partial Drosera chitinase has 77% and 73% identity to chitinase 1 (Nkchit1b) and chitinase 2 (Nkchit2b), respectively, while Dionea chitinase shows 73% and 67% identity, respectively. Isolation of the 5′ and 3′ sequences of the genes will provide a more accurate estimation of the resemblance of the chitinases from the different sources.

### Example 20

### Kinetic Properties of Trap Soup Chitinase

To further characterize biochemically the chitinase activity present in the Nepenthes trap soup, the kinetic properties of the soup chitinase was compared with that of recombinant Serratia marcescens chitinase.

Sterile trap soup was collected from closed traps and concentrated 4.8 fold by speedvaccing. Serratia marcescens chitinase (lyophilized powder) was purchased from Sigma (C1650) and dissolved in H₂O.

Due to the very low amount of chitinase protein/s present in the trap soup, the exact protein amount could not be detected by the regular methods (see Example 6). Thus, to estimate the enzyme amount used for activity assays the amount of the ˜32-35 kDa band which corresponded to the deduced sizes of the isolated cDNAs of Nepenthes chitinase/s protein concentration was determined by SDS-PAGE and silver staining. Similarly, the amount of the commercial Serratia marcescens chitinase was also determined.

FIG. 1. shows the approximate quantitation of the Nepenthes and Serratia chitinases on 12% SDS-PAGE gels after silver staining. BSA (˜66 kDa) and Carbonic anhydrase (29 kDa) were used for quantity calibration of Serratia (˜58 kDa) and Nepenthes (˜32-35 kDa) chitinases, respectively. The amount of chitinase used for the activity assays was estimated to be approximately 100 ng (in 5 μl) for Serratia. In case of Nepenthes chitinase, there was only a slight band at 32-35 kDa which could represent the enzyme and therefore, the amount of chitinase was estimated to be maximally in the range of 20-30 ng (in 30 μl).

To determine the kinetic profile of the enzymes in both preparations chitinase activity assay was performed in the presence of increasing substrate (tetramer: p-nitrophenyl-β-D-N,N′,N″-triacetyl-chitotriose, Sigma N8638) concentrations and p-nitrophenol release was determined.

FIG. 2. shows that the Nepenthes chitinase seems to have a higher Kₘ than the Serratia enzyme, but its activity is still linearly correlated with substrate concentration, while Serratia chitinase reached maximal activity already at substrate concentration of 9 μg/assay. Furthermore, under repeated assays the Nepenthes enzyme showed a relationship between Vo and [S] that differs from the normal Michaelis-Menten behavior (data not shown). A sigmoid (rather than hyperbolic) saturation curve, which is characteristic for allosteric enzymes, was observed. This sigmoid kinetic behavior generally reflects cooperative interactions between multiple protein subunits [Lehninger et al., (1993) Principles in Biochemistry 229-233]. It has previously been shown that the presence of β-mercaptoethanol, in addition to SDS, inactivates the soup chitinase while it does not affect leaf chitinase activity (Example 5.). These results already suggested that the trap soup chitinase might be a dimer held together by inter-molecular disulphide bonds. Although most plant chitinases are active as monomers of ˜25-35 kDa, a dimeric chitinase has been identified in the seeds of Job's tears (reviewed by L. S. Graham and M. B. Sticklen, 1994). A clear conclusion about the nature of the Nepenthes chitinase cannot be made since more than one type of chitinase might be present in the trap soup whose activity was studied above. Therefore, final characterization will be performed only with the purified enzymes, following their separate expression in transgenic plants.

### REFERENCES CITED

(Additional references are cited in the text)

Andersen M D, Jensen A, Rebertus J D, Leah R. and Skriver K (1997) Biochem. J. 322, 815-822.

Ary M B, Richardson M ans Shrewry P R (1989) Biochem. Biophys. ACTA 993, 260-266.

Bolar J P, Norelli J L, Wong K-W, Hayes C K, Harman G E and Aldwinckle H S (2000) Phytopathology 90, 72-77.

Broglie K, Chet I, Holliday M, Cressman R, Biddle P, Knowlton S, Mauvais C J and Broglie R (1991) Science 254, 1194-1197.

Carr, J. P. and Klessing, D. F. (1989) In: Genetic Engineering Principles and Methods, 11, 65-109, Plenum Press, New York.

Chrispeels M J and Raikhel N V (1992) Cell 68, 613-616.

Collinge, D. B, Kragh, K. M., Mikkelsen, J. D., Nielsen, K. K., Rasmussen, U. and Vad, K. (1993) Plant J. 3, 31-40.

De Jong A J, Cordewener J, Lo Schiavo F, Terzi M, Vandeckerckhove J, Van Kammen A and De Vries S (1992) Cell 4, 425-433.

Ferrando A, Koncz-Kalman Z, Farras R, Tiburcio A, Schell J and Koncz C (2001) Nucl. Acids Res. 29, 3685-3693.

Graham, L. S. and Sticklen, M. B. (1994) Can. J. Bot. 72, 1057-1083.

Hamel F, Boivin R, Tremblay C and Bellemare G (1997) J. Mol. Evol. 44, 614-624.

Hart P J, Monzingo A F, Ready M P, Ernst SR and Robertus J D (1993) J Mol Biol 229, 189-193.

Hohn, B. and Collins, J. (1980) Gene 11, 291-298.

Jach G, Gornhardt B, Mundy J, Logemann J, Pinsdorf E, Leah R, Schell J and Maas C (1995) The Plant J. 8(1), 97-109.

Jones D G J, Grady K L, Suslow T V and Bedbrook J R (1986) The EMBO J. 5, 467-473.

Legrand, M., Kauffman, S., Geoffroy, P. and Fritig, B. (1987) Proc. Natl. Acad. Sci. USA, 84, 6750-6754.

Lerner D R and Raikhel N V (1992) J. Biol. Chem. 267, 11085-11091.

Lorito M, Woo S L, Fernandez I G, Colucci G, Harman G E, Pintor-Toro J A, Filippone E, Muccifora S, Lawrence C B, Zoina A, Tuzun S and Scala F (1998). Proc. Natl. Acad. Sci. USA 95, 7860-7865.

Margis-Pinhiero M, Metz-Boutigue M H, Awade A, de Tapia M, le Ret M and Burkard G (1991) Plant Mol. Biol. 17, 243-253.

Newman T C, Ohme-Takagi M, Taylor C B and Green P J (1993) The Plant Cell 5, 701-714.

Owen T P and Lennon K A (1999) American J. of Bot. 86(10), 1382-1390.

Punja Z K and Raharjo S H T (1996) Plant Dis. 80, 99-105.

Schulze W. Frommer W B and Ward J M (1999) The Plant J. 17(6), 637-646.

Song H K, Hwang K Y, Kim K K and Suh S W (1993) Proteins:Structure, Function and Genetics 17, 107-109.

Swegle M, Kramer K J and Muthuksishnan S (1992) Plant Physiol. 99, 1009-1014.

Vad K, Mikkelsen J and Collinge D B (1991) Planta 184, 24-29.

Van Damme E J M, Willens P, Torrekens S, Van Leuven F and Peumans W J (1993) Physiol. Plant. 87, 177-186.

Zhu Q, Maher E A, Masoud A, Dixon R A and Lamb C J (1994) Bio/Technology 12, 807-812.

What is claimed is:

1. An enzymatic composition comprising at least one protein isolated from a tissue or soup of a carnivorous plant, said at least one protein being characterized by an endo-chitinase activity.

2. The enzymatic composition of claim 1, wherein said at least one protein is characterized by a pI below 10.

3. The enzymatic composition of claim 1, wherein said at least one protein is not reactive with an anti ChiAII polyclonal antibody.

4. The enzymatic composition of claim 1, wherein said at least one protein does not exhibit endo-chitinase activity following exposure to reducing conditions.

5. The enzymatic composition of claim 1, wherein said at least one protein is characterized by an apparent molecular weight of about 32.7 kDa as determined by 12% SDS-PAGE.

6. The enzymatic composition of claim 1, wherein said at least one protein is characterized by an apparent molecular weight of about 36 kDa as determined by 12% SDS-PAGE.

7. A pharmaceutical composition comprising as an active ingredient the enzymatic composition of claim 1 and a pharmaceutically acceptable carrier or diluent.

8. The enzymatic composition of claim 1, wherein said at least one protein is characterized by an anti-fungal activity.

9. The enzymatic composition of claim 8, wherein said anti-fungal activity is fungicidal activity.

10. The enzymatic composition of claim 8, wherein said anti-fungal activity is anti Candida albicans activity.

11. A composition for disinfesting chitin-containing pathogens, the composition comprising as an active ingredient the enzymatic composition of claim 1 and a carrier or diluent.

12. An agronomical composition comprising as an active ingredient the enzymatic composition of claim 1 and an agronomically acceptable carrier.

13. The enzymatic composition of claim 1, wherein said at least one protein is at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

14. The enzymatic composition of claim 13, wherein said at least one protein is as set forth in SEQ ID NOs: 5, 6, 7 or 8 or active portions thereof.

15. The enzymatic composition of claim 1, wherein said tissue is trap tissue and/or leaf tissue.

16. The enzymatic composition of claim 1, wherein said soup is trap soup.

17. The enzymatic composition of claim 1, wherein said carnivorous plant is selected from the group consisting of Nepenthes ssp., Drosera sp., Dionea sp. and Sarracenia sp.

18. An enzymatic composition comprising a protein extract of a tissue or soup of a carnivorous plant, wherein said protein extract includes at least one protein exhibiting endo-chitinase activity.

19. The enzymatic composition of claim 18, wherein said at least one protein is characterized by a pI below 10.

20. The enzymatic composition of claim 18, wherein said at least one protein is not reactive with an anti ChiAII polyclonal antibody.

21. The enzymatic composition of claim 18, wherein said at least one protein does not exhibit endo-chitinase activity following exposure to reducing conditions.

22. The enzymatic composition of claim 18, wherein said at least one protein is characterized by an apparent molecular weight of about 32.7 kDa as determined by 12% SDS-PAGE.

23. The enzymatic composition of claim 18, wherein said at least one protein is characterized by an apparent molecular weight of about 36 kDa as determined by 12% SDS-PAGE.

24. A pharmaceutical composition comprising as an active ingredient the enzymatic composition of claim 18 and a pharmaceutically acceptable carrier or diluent.

25. The enzymatic composition of claim 18, wherein said at least one protein is characterized by an anti-fungal activity.

26. The enzymatic composition of claim 25, wherein said anti-fungal activity is fungicidal activity.

27. The enzymatic composition of claim 25, wherein said anti-fungal activity is anti Candida albicans activity.

28. A composition for disinfesting chitin-containing pathogens, the composition comprising as an active ingredient the enzymatic composition of claim 18 and a carrier or diluent.

29. An agronomical composition comprising as an active ingredient the enzymatic composition of claim 18 and an agronomically acceptable carrier.

30. The enzymatic composition of claim 18, wherein said at least one protein is at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

31. The enzymatic composition of claim 30, wherein said at least one protein is as set forth in SEQ ID NOs: 5, 6, 7 or 8 or active portions thereof.

32. The enzymatic composition of claim 18, wherein said tissue is trap tissue and/or leaf tissue.

33. The enzymatic composition of claim 18, wherein said soup is trap soup.

34. The enzymatic composition of claim 18, wherein said carnivorous plant is selected from the group consisting of Nepenthes ssp., Drosera sp., Dionea sp. and Sarracenia sp.

35. An isolated nucleic acid comprising a polynucleotide sequence encoding a polypeptide having an endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

36. The isolated nucleic acid of claim 35, wherein said polynucleotide sequence is selected from the group consisting of SEQ ID NOs: 1, 2, 3, 4 and 48 or active portions thereof.

37. The isolated nucleic acid of claim 35, wherein said polypeptide is selected from the group consisting of SEQ ID NOs: 5, 6, 7 and 8 or active portions thereof.

38. The isolated nucleic acid of claim 359, wherein said polynucleotide sequence is selected from the group consisting of a genomic polynucleotide sequence, a complementary polynucleotide sequence and a composite polynucleotide sequence.

39. A nucleic acid construct comprising the isolated nucleic acid of claim 35.

40. A host cell comprising the nucleic acid construct of claim 39.

41. An isolated nucleic acid comprising a polynucleotide sequence selected from the group consisting of SEQ ID NOs: 1, 2, 3, 4 and 48.

42. The isolated nucleic acid of claim 41, wherein said polynucleotide sequence is selected from the group consisting of a genomic polynucleotide sequence, a complementary polynucleotide sequence and a composite polynucleotide sequence.

43. A nucleic acid construct comprising the isolated nucleic acid of claim 41.

44. A host cell comprising the nucleic acid construct of claim 43.

45. An isolated nucleic acid comprising a polynucleotide sequence encoding a polypeptide having an endo-chitinase activity and including a signal peptide of at least 30 amino acids.

46. The isolated nucleic acid of claim 45, wherein said signal peptide is for protein secretion.

47. The isolated nucleic acid of claim 45, wherein said polynucleotide sequence is set forth in SEQ ID NOs: 1 or 48 or active portions thereof.

48. The isolated nucleic acid of claim 45, wherein said polypeptide is set forth in SEQ ID NO: 5 or active portions thereof.

49. The isolated nucleic acid of claim 45, wherein said polynucleotide sequence is selected from the group consisting of a genomic polynucleotide sequence, a complementary polynucleotide sequence and a composite polynucleotide sequence.

50. The isolated nucleic acid of claim 45, wherein said signal peptide is set forth in SEQ ID NO: 47.

51. The isolated nucleic acid of claim 45, wherein said polynucleotide sequence is selected from the group consisting of a genomic polynucleotide sequence, a complementary polynucleotide sequence and a composite polynucleotide sequence.

52. A nucleic acid construct comprising the isolated nucleic acid of claim 45.

53. A host cell comprising the nucleic acid construct of claim 45.

54. An isolated nucleic acid comprising at least 67% identical with SEQ ID NO: 1 or at least 75% identical with SEQ ID NO: 2 as determined using the BestFit software of the Wisconsin sequence analysis package, utilizing the Smith and Waterman algorithm, where gap weight equals 50, length weight equals 3, average match equals 10 and average mismatch equals −9.

55. A nucleic acid construct comprising the isolated nucleic acid of claim 54.

56. A host cell comprising the nucleic acid construct of claim 55.

57. The isolated nucleic acid of claim 54, wherein said polynucleotide sequence is selected from the group consisting of a genomic polynucleotide sequence, a complementary polynucleotide sequence and a composite polynucleotide sequence

58. An oligonucleotide of at least 17 bases specifically hybridizable with an isolated nucleic acid set forth in SEQ ID NO: 1, 2, 3, 4 or 48.

59. A pair of oligonucleotides each of at least 17 bases specifically hybridizable with SEQ ID NO: 1, 2, 3, 4 or 48 in an opposite orientation so as to direct specific amplification of a portion thereof in a nucleic acid amplification reaction.

60. An isolated polypeptide having endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

61. A pharmaceutical composition comprising as an active ingredient the isolated polypeptide of claim 60 and a pharmaceutically acceptable carrier or diluent.

62. The pharmaceutical composition of claim 61, wherein said pharmaceutically acceptable carrier or diluent is formulated for topical application, or oral administration.

63. A composition for disinfesting chitin-containing pathogens, the composition comprising as an active ingredient the enzymatic composition of claim 60 and a carrier or diluent.

64. An agronomical composition comprising as an active ingredient the enzymatic composition of claim 60 and an agronomically acceptable carrier.

65. An isolated polypeptide selected from the group consisting of SEQ ID NOs: 5, 6, 7 and 8 or active portions thereof.

66. A pharmaceutical composition comprising as an active ingredient the isolated polypeptide of claim 65 and a pharmaceutically acceptable carrier or diluent.

67. A composition for disinfesting chitin-containing, the composition comprising as an active ingredient the enzymatic composition of claim 65 and a carrier or diluent.

68. An agronomical composition comprising as an active ingredient the enzymatic composition of claim 65 and an agronomically acceptable carrier.

69. A method of treating an individual having a disease or a condition associated with a chitin-containing pathogen, the method comprising administering to the individual a therapeutically effective amount of a pharmaceutical composition including as an active ingredient a protein extract derived from a trap soup or a trap tissue of a carnivorous plant, said protein extract including at least one protein exhibiting endo-chitinase activity.

70. The method of claim 69, wherein said at least one protein is characterized by a pI below 10.

71. The method on of claim 69, wherein said at least one protein is not reactive with an anti ChiAII polyclonal antibody.

72. The method of claim 69, wherein said at least one protein does not exhibit endo-chitinase activity following exposure to reducing conditions.

73. The method of claim 69, wherein said at least one protein is characterized by an apparent molecular weight of about 32.7 kDa as determined by 12% SDS-PAGE.

74. The method of claim 69, wherein said at least one protein is characterized by an apparent molecular weight of about 36 kDa as determined by 12% SDS-PAGE.

75. The method of claim 69, wherein said pharmaceutical composition further includes a pharmaceutically acceptable carrier or diluent.

76. The method of claim 69, wherein said at least one protein is at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

77. The method of claim 76, wherein said at least one protein is as set forth in SEQ ID NOs: 5, 6, 7 or 8 or active portions thereof.

78. The method of claim 69, wherein said tissue is trap tissue and/or leaf tissue.

79. The method of claim 69, wherein said soup is trap soup.

80. The method of claim 69, wherein said carnivorous plant is selected from the group consisting of Nepenthes ssp., Drosera sp., Dionea sp. and Sarracenia sp.

81. A method of generating a pharmaceutical composition useful for treating a disease or a condition associated with a chitin-containing pathogen, the method comprising:
(a) extracting a protein fraction from a trap soup or a trap tissue of a carnivorous plant, said protein fraction exhibiting endo-chitinase activity; and
(b) mixing said protein fraction with a pharmaceutically acceptable carrier or diluent, thereby generating the pharmaceutical composition useful for treating the disease or the condition associated with the chitin-containing pathogen.

82. The method of claim 81, wherein said carnivorous plant is selected from the group consisting of Nepenthes ssp., Drosera sp., Dionea sp. and Sarracenia sp.

83. The method of claim 81, further comprising exposing said trap soup or trap tissue of said carnivorous plant to chitin prior to (a).

84. A method of reducing susceptibility of a plant to a chitin-containing pathogen, the method comprising expressing within the plant an exogenous polypeptide having an endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

85. The method of claim 84, wherein said exogenous polypeptide is selected from the group consisting of SEQ ID NOs: 5, 6, 7 and 8 or active portions thereof.

86. A method of reducing susceptibility of a plant to a chitin-containing pathogen, the method comprising exposing the plant to a composition including as an active ingredient a protein extract derived from a soup or a tissue of a carnivorous plant, said protein extract including at least one protein exhibiting endo-chitinase activity.

87. The method of claim 86, wherein said at least one protein is characterized by a pI below 10.

88. The method on of claim 86, wherein said at least one protein is not reactive with an anti ChiAII polyclonal antibody.

89. The method of claim 86, wherein said at least one protein does not exhibit endo-chitinase activity following exposure to reducing conditions.

90. The method of claim 86, wherein said at least one protein is characterized by an apparent molecular weight of about 32.7 kDa as determined by 12% SDS-PAGE.

91. The method of claim 86, wherein said at least one protein is characterized by an apparent molecular weight of about 36 kDa as determined by 12% SDS-PAGE.

92. The method of claim 86, wherein said composition further includes a carrier or diluent.

93. The method of claim 86, wherein said at least one protein is at least 70% identical to SEQ ID NO: 5 at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

94. The method of claim 93, wherein said at least one protein is as set forth in SEQ ID NOs: 5, 6, 7 or 8 or active portions thereof.

95. The method of claim 86, wherein said tissue is trap tissue and/or leaf tissue.

96. The method of claim 86, wherein said soup is trap soup.

97. The method of claim 86, wherein said carnivorous plant is selected from the group consisting of Nepenthes ssp., Drosera sp., Dionea sp. and Sarracenia sp.

98. A method of isolating polypeptides exhibiting a high endo-chitinase activity, the method comprising:
(a) preparing a protein extract from a trap tissue or a trap soup of a carnivorous plant; and
(b) isolating from said protein extract a chitinase active fraction, thereby isolating polypeptides exhibiting high endo-chitinase activity.

99. The method of claim 98, further comprising exposing said trap tissue or said trap soup to chitin prior to (a).

100. The method of claim 98, wherein said carnivorous plant is selected from the group consisting of Nepenthes ssp., Drosera sp., Dionea sp. and Sarracenia sp.

101. A method of reducing susceptibility of a plant to cold damage, the method comprising expressing within a plurality of plants an exogenous polypeptide having an endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

102. The method of claim 101, wherein said exogenous polypeptide is selected from the group consisting of SEQ ID NOs: 5, 6, 7 and 8 or active portions thereof.

103. A method of reducing susceptibility of a plant to cold damage, the method comprising, exposing a plurality of plants to a composition including as an active ingredient a protein extract derived from a soup or tissue of a carnivorous plant, said protein extract including at least one protein exhibiting endo-chitinase activity.

104. The method of claim 103, wherein said at least one protein is characterized by a pI below 10.

105. The method on of claim 103, wherein said at least one protein is not reactive with an anti ChiAII polyclonal antibody.

106. The method of claim 103, wherein said at least one protein does not exhibit endo-chitinase activity following exposure to reducing conditions.

107. The method of claim 103, wherein said at least one protein is characterized by an apparent molecular weight of about 32.7 kDa as determined by 12% SDS-PAGE.

108. The method of claim 103, wherein said at least one protein is characterized by an apparent molecular weight of about 36 kDa as determined by 12% SDS-PAGE.

109. The method of claim 103, wherein said composition further includes a carrier or diluent.

110. The method of claim 103, wherein said at least one protein is at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

111. The method of claim 110, wherein said at least one protein is as set forth in SEQ ID NOs: 5, 6, 7 or 8 or active portions thereof.

112. The method of claim 103, wherein said tissue is trap tissue and/or leaf tissue.

113. The method of claim 103, wherein said soup is trap soup.

114. The method of claim 103, wherein said carnivorous plant is selected from the group consisting of Nepenthes ssp., Drosera sp., Dionea sp. and Sarracenia sp.

115. A plant, a plant tissue or a plant seed comprising an exogenous polynucleotide sequence encoding a polypeptide having an endo-chitinase activity and being at least 70% identical to SEQ ID NO: 5, at least 75% identical to SEQ ID NO: 6, at least 81% identical to SEQ ID NO: 7 or at least 77% identical to SEQ ID NO: 8 as determined using the BestFit software of the Wisconsin sequence analysis package, utilizing the Smith and Waterman algorithm, where the gap creation equals 8 and the gap extension penalty equals 2.

116. The plant, the plant tissue or the plant seed of claim 115, wherein said polynucleotide sequence is selected from the group consisting of SEQ ID NOs: 1, 2, 3, 4 and 48 or active portions thereof.

117. The plant, the plant tissue or the plant seed of claim 115, wherein said polypeptide is selected from the group consisting of SEQ ID NOs: 5, 6, 7 and 8 or active portions thereof.

118. The plant, the plant tissue or the plant seed of claim 115, wherein said polynucleotide sequence is selected from the group consisting of a genomic polynucleotide sequence, a complementary polynucleotide sequence and a composite polynucleotide sequence.

119. An isolated nucleic acid comprising a polynucleotide sequence encoding a polypeptide having an endo-chitinase activity and including a proline rich region having at least 10 and no more than 15 proline amino acids.

120. The isolated nucleic acid of claim 119, wherein said proline rich region includes 6 putative glycosylation sites.

121. The isolated nucleic acid of claim 119, wherein said polynucleotide sequence is set forth in SEQ ID NOs: 1 or 48 or active portions thereof.

122. The isolated nucleic acid of claim 119, wherein said polypeptide is set forth in SEQ ID NO: 5 or active portions thereof.

123. The isolated nucleic acid of claim 119, wherein said polynucleotide sequence is selected from the group consisting of a genomic polynucleotide sequence, a complementary polynucleotide sequence and a composite polynucleotide sequence.

124. The isolated nucleic acid of claim 119, wherein said proline rich region is set forth in SEQ ID NO: 49.

125. A nucleic acid construct comprising the isolated nucleic acid of claim 119.

126. A host cell comprising the nucleic acid construct of claim 125.
